SEQUENCE LISTING (60 sequences)

SEQ ID NO: 1 - DNA, 1084 bp, Homo sapiens
  Human OX40 cDNA
cgaggatgtgcgtgggggctcggcggctgggccgcgggccgtgtgcggctctgctcctcctgggcctggggctgagcaccgtgacggggctccactgtgtcggggacacctaccccagcaacgaccggtgctgccacgagtgcaggccaggcaacgggatggtgagccgctgcagccgctcccagaacacggtgtgccgtccgtgcgggccgggcttctacaacgacgtggtcagctccaagccgtgcaagccctgcacgtggtgtaacctcagaagtgggagtgagcggaagcagctgtgcacggccacacaggacacagtctgccgctgccgggcgggcacccagcccctggacagctacaagcctggagttgactgtgccccctgccctccagggcacttctccccaggcgacaaccaggcctgcaagccctggaccaactgcaccttggctgggaagcacaccctgcagccggccagcaatagctcggacgcaatctgtgaggacagggaccccccagccacgcagccccaggagacccagggccccccggccaggcccatcactgtccagcccactgaagcctggcccagaacctcacagggaccctccacccggcccgtggaggtccccgggggccgtgcggttgccgccatcctgggcctgggcctggtgctggggctgctgggccccctggccatcctgctggccctgtacctgctccggagggaccagaggctgccccccgatgcccacaagccccctgggggaggcagtttccggacccccatccaagaggagcaggccgacgcccactccaccctggccaagatctgacctgggcccaccaaggtggacgctgggccccgccaggctggagcccggagggtctgctgggcgagcagggcaggtgcaggccgcctgccccgccacgctcctgggccaactctgcaccgttctaggtgccgatggctgcctccggctctctgcttacgtatgccatgcatacctcctgccccgcgggaccacaataaaaaccttggcagacgggagtctccgaccggcaaaaaaaaaaaaaaaaa

SEQ ID NO: 2 - protein, 277 aa, Homo sapiens
  Human OX40 Amino Acid Sequence
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 3 - DNA, 33 bp, Artificial Sequence
  OLIGONUCLEOTIDE PRIMER FOR OX40
cccaagctttaccccagcaacgaccggtgctgc

SEQ ID NO: 4 - DNA, 29 bp, Artificial Sequence
  OLIGONUCLEOTIDE PRIMER FOR OX40
cgcctcgaggacctccacgggccgggtgg

SEQ ID NO: 5 - DNA, 30 bp, Artificial Sequence
  OLIGONUCLEOTIDE PRIMER FOR OX40
caccatgtgcgtgggggctcggcggctggg

SEQ ID NO: 6 - DNA, 31 bp, Artificial Sequence
  OLIGONUCLEOTIDE PRIMER FOR OX40
gatcttggccagggtggagtgggcgtcggcc

SEQ ID NO: 7 - protein, 111 aa, Artificial Sequence
  VARIABLE LIGHT CHAIN REGION OF MURINE ANTI-OX40 ANTIBODY B66
DIVLTQSPASLAVSLGQRATISCKASQTVDYDGDSYMHWYQQKPGQPPKLLIYAASNLESGIPARFSGSGSGTDFTLNIHPVEEEDAATYYCQQSNEDPYTFGGGTKLEIK

SEQ ID NO: 8 - protein, 111 aa, Artificial Sequence
  VARIABLE LIGHT CHAIN REGION OF MURINE ANTI-OX40 ANTIBODY A10
DIVLTQSPASLAVSLGQRATISCKASQSVDYDGDSYMNWYQQKPGQPPKLLIYAASNLESGIPARFSGSGSGTDFTLNIHPVEEEDATTYYCHQSNEDPYTFGGGTKLEIK

SEQ ID NO: 9 - protein, 111 aa, Artificial Sequence
  VARIABLE LIGHT CHAIN REGION OF HUMANIZED ANTI-OX40 ANTIBODY A10LC(TH)hu336
DIVLTQSPTSLAVSLGQRATISCKASQTVDYDGDSYMHWYQQKPGQPPKLLIYAASNLESGVPDRFSGSGSGTDFTLTISSLQAEDVAVYYCHQSNEDPWTFGQGTKVEIK

SEQ ID NO: 10 - protein, 120 aa, Artificial Sequence
  VARIABLE HEAVY CHAIN REGION OF MURINE ANTI-OX40 ANTIBODY A10
QVQLQQPGSVLVRPGASVKLSCKASGYTFTSYWMHWVKQRPGQGLEWIGEIHPKSGNSNYNEKFKGRATLTVDTSSSTAYVDLSSLTSEDSAVYYCARGPEYSNFWFAYWGQGTLVTVSA

SEQ ID NO: 11 - protein, 120 aa, Artificial Sequence
  VARIABLE HEAVY CHAIN REGION OF HUMANIZED ANTI-OX40 ANTIBODY A10LC(TH)hu336
QVQLVQSGAEVKKPGASVKVSCKASGYTFTSYWMHWVRQAPGQGLEWMGEIHPKSGNSNYNEKFKGRVTMTRDTSTSTVYMELSSLRSEDTAVYYCARGPEYSNFWFAYWGQGTLVTVSS

SEQ ID NO: 12 - protein, 15 aa, Artificial Sequence
  CDR-L1 OF MURINE ANTI-OX40 ANTIBODY B66
KASQTVDYDGDSYMH

SEQ ID NO: 13 - protein, 7 aa, Artificial Sequence
  CDR-L2 OF ANTI-OX40 ANTIBODY A10 AND B66
AASNLES

SEQ ID NO: 14 - protein, 9 aa, Artificial Sequence
  CDR-L3 OF ANTI-OX40 ANTIBODY B66
QQSNEDPYT

SEQ ID NO: 15 - protein, 15 aa, Artificial Sequence
  CDR-L1 OF ANTI-OX40 ANTIBODY A10
KASQSVDYDGDSYMN

SEQ ID NO: 16 - protein, 9 aa, Artificial Sequence
  CDR-L3 OF ANTI-OX40 ANTIBODY A10
HQSNEDPWT

SEQ ID NO: 17 - protein, 5 aa, Artificial Sequence
  CDR-H1 OF ANTI-OX40 ANTIBODY A10 AND B66
SYWMH

SEQ ID NO: 18 - protein, 17 aa, Artificial Sequence
  CDR-H2 OF ANTI-OX40 ANTIBODY A10 AND B66
EIHPKSGNSNYNEKFKG

SEQ ID NO: 19 - protein, 11 aa, Artificial Sequence
  CDR-H3 OF ANTI-OX40 ANTIBODY A10 AND B66
GPEYSNFWFAY

SEQ ID NO: 20 - DNA, 333 bp, Artificial Sequence
  VARIABLE LIGHT CHAIN REGION OF MURINE ANTI-OX40 ANTIBODY A10
gacattgtgctgacccaatctccaacttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctacaacctattactgtcaccagagtaatgaggatccgtggacgttcggtggaggcaccaagctggaaatcaaa

SEQ ID NO: 21 - DNA, 333 bp, Artificial Sequence
  VARIABLE LIGHT CHAIN REGION OF MURINE ANTI-OX40 ANTIBODY B66
gacattgtgctgacccaatctccagcttctttggctgtgtctctagggcagagggccaccatctcctgcaaggccagccaaactgttgattatgatggtgacagttatatgcattggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtacacgttcggaggggggaccaagctggaaataaaa

SEQ ID NO: 22 - DNA, 333 bp, Artificial Sequence
  VARIABLE LIGHT CHAIN REGION OF HUMANIZED ANTI-OX40 ANTIBODY A10B & A10E
gacatcgtgatgacccagagcccagacagcctggccgtgagcctgggcgagcgcgccaccatcaactgcaaggccagccagagcgtggactacgacggcgacagctacatgaactggtaccagcagaagccaggccagccaccaaagctgctgatctacgccgccagcaacctggagagcggcgtgccagaccgcttcagcggcagcggcagcggcaccgacttcaccctgaccatcagcagcctgcaggccgaggacgtggccgtgtactactgccaccagagcaacgaggacccatggaccttcggccagggcaccaaggtggagatcaag

SEQ ID NO: 23 - DNA, 333 bp, Artificial Sequence
  VARIABLE LIGHT CHAIN REGION OF HUMANIZED ANTI-OX40 ANTIBODY A10A & A10D
gacatcgtgatgacccagagcccagacagcctggccgtgagcctgggcgagcgcgccaccatcaactgcaaggccagccagagcgtggactacgacggcgacagctacatgcactggtaccagcagaagccaggccagccaccaaagctgctgatctacgccgccagcaacctggagagcggcgtgccagaccgcttcagcggcagcggcagcggcaccgacttcaccctgaccatcagcagcctgcaggccgaggacgtggccgtgtactactgccaccagagcaacgaggacccatggaccttcggccagggcaccaaggtggagatcaag

SEQ ID NO: 24 - DNA, 333 bp, Artificial Sequence
  VARIABLE LIGHT CHAIN REGION OF HUMANIZED ANTI-OX40 ANTIBODY A10(TH)HU336F & A10C
gacatcgtgatgacccagagcccagacagcctggccgtgagcctgggcgagcgcgccaccatcaactgcaaggccagccagaccgtggactacgacggcgacagctacatgcactggtaccagcagaagccaggccagccaccaaagctgctgatctacgccgccagcaacctggagagcggcgtgccagaccgcttcagcggcagcggcagcggcaccgacttcaccctgaccatcagcagcctgcaggccgaggacgtggccgtgtactactgccaccagagcaacgaggacccatggaccttcggccagggcaccaaggtggagatcaag

SEQ ID NO: 25 - DNA, 360 bp, Artificial Sequence
  VARIABLE HEAVY CHAIN REGION OF MURINE ANTI-OX40 ANTIBODY A10
caggtccaactgcagcagcctgggtctgtgctggtgaggcctggagcttcagtgaagctgtcctgcaaggcttctggctacaccttcaccagctactggatgcattgggtgaagcagaggcctggacaaggccttgagtggattggagagattcatcctaaaagtggtaatagtaactacaatgagaagttcaagggcagggccacactgactgtagacacatcctccagcacagcctacgtggatcttagcagcctgacatctgaggactctgcggtctattactgtgcaagaggtccggaatatagtaatttttggtttgcttattggggccaaggaactctggtcactgtctctgca

SEQ ID NO: 26 - DNA, 360 bp, Artificial Sequence
  VARIABLE HEAVY CHAIN REGION OF HUMANIZED ANTI-OX40 ANTIBODY A10(TH)HU336F & A10D &A10E
caggtgcagctggtgcagagcggcgccgaggtgaagaagccaggcgccagcgtgaaggtgagctgcaaggccagcggctacaccttcaccagctactggatgcactgggtgcgccaggccccaggccagggcctggagtggatgggcgagatccacccaaagagcggcaacagcaactacaacgagaagttcaagggccgcgtgaccatgacccgcgacaccagcaccagcaccgtgtacatggagctgagcagcctgcgcagcgaggacaccgccgtgtactactgcgcccgcggcccagagtacagcaacttctggttcgcctactggggccagggcaccctggtgaccgtgagcagc

SEQ ID NO: 27 - DNA, 360 bp, Artificial Sequence
  VARIABLE HEAVY CHAIN REGION OF HUMANIZED ANTI-OX40 ANTIBODY A10L70 & A10A-C
caggtgcagctggtgcagagcggcgccgaggtgaagaagccaggcgccagcgtgaaggtgagctgcaaggccagcggctacaccttcaccagctactggatgcactgggtgcgccaggccccaggccagggcctggagtggatgggcgagatccacccaaagagcggcaacagcaactacaacgagaagttcaagggccgcgtgaccctgacccgcgacaccagcaccagcaccgtgtacatggagctgagcagcctgcgcagcgaggacaccgccgtgtactactgcgcccgcggcccagagtacagcaacttctggttcgcctactggggccagggcaccctggtgaccgtgagcagc

SEQ ID NO: 28 - protein, 111 aa, Homo sapiens
  VARIANT 24, 25, 26, 27, 28, 29, 30, 31, 32, 33, 34, 35, 36, 37,38, 54, 55, 56, 57, 58, 59, 60, 93, 94, 95, 96, 97, 98, 99, 100,101: Xaa = Any Amino Acid
  Human template Variable Light Chain IGKV4-1/IGKJ1
DIVMTQSPDSLAVSLGERATINCXXXXXXXXXXXXXXXWYQQKPGQPPKLLIYXXXXXXXGVPDRFSGSGSGTDFTLTISSLQAEDVAVYYCXXXXXXXXXFGQGTKVEIK

SEQ ID NO: 29 - protein, 120 aa, Homo sapiens
  VARIANT 31, 32, 33, 34, 35, 50, 51, 52, 53, 54, 55, 56, 57, 58,59, 60, 61, 62, 63, 64, 65, 66, 99, 100, 101, 102, 103, 104,105, 106, 107, 108, 109: Xaa = Any Amino Acid
  Human Template Heavy Chain Region IGHV1-45/IGHJ4
QVQLVQSGAEVKKPGASVKVSCKASGYTFTXXXXXWVRQAPGQGLEWMGXXXXXXXXXXXXXXXXXRVTMTRDTSTSTVYMELSSLRSEDTAVYYCARXXXXXXXXXXXWGQGTLVTVSS

SEQ ID NO: 30 - protein, 132 aa, Artificial Sequence
  VARIANT 31, 32, 33, 34, 35, 36, 37, 52, 53, 54, 55, 56, 57, 58,59, 60, 61, 62, 63, 64, 65, 66, 67, 68, 69, 70, 103, 104, 105,106, 107, 108, 109, 110, 111, 112, 113, 114, 115, 116, 117,118, 119, 120, 121: Xaa = Any Amino Acid
  HEAVY CHAIN FRAMEWORK REGION
QVQLVQSGAEVKKPGASVKVSCKASGYTFTXXXXXXXWVRQAPGQGLEWMGXXXXXXXXXXXXXXXXXXXRVTITADTSTSTAYMELSSLRSEDTAVYYCARXXXXXXXXXXXXXXXXXXXWGQGTLVTVSS

SEQ ID NO: 31 - protein, 132 aa, Artificial Sequence
  VARIANT 26, 27, 28, 29, 30, 31, 32, 33, 34, 35, 36, 37, 51, 52,53, 54, 55, 56, 57, 58, 59, 60, 61, 62, 63, 64, 65, 66, 67,68, 69, 70, 103, 104, 105, 106, 107, 108, 109, 110, 111,112, 113, 114, 115, 116, 117, 118, 119, 120, 121: Xaa = Any Amino Acid
  HEAVY CHAIN FRAMEWORK REGION
QVQLVQSGAEVKKPGASVKVSCKASXXXXXXXXXXXXWVRQAPGQGLEWMXXXXXXXXXXXXXXXXXXXXRVTITADTSTSTAYMELSSLRSEDTAVYYCARXXXXXXXXXXXXXXXXXXXWGQGTLVTVSS

SEQ ID NO: 32 - protein, 132 aa, Artificial Sequence
  VARIANT 26, 27, 28, 29, 30, 31, 32, 33, 34, 35, 36, 37, 51, 52,53, 54, 55, 56, 57, 58, 59, 60, 61, 62, 63, 64, 65, 66, 67,68, 69, 70, 102, 103, 104, 105, 106, 107, 108, 109, 110,111, 112, 113, 114, 115, 116, 117, 118, 119, 120, 121: Xaa = Any Amino Acid
  HEAVY CHAIN FRAMEWORK REGION
QVQLVQSGAEVKKPGASVKVSCKASXXXXXXXXXXXXWVRQAPGQGLEWMXXXXXXXXXXXXXXXXXXXXRVTITADTSTSTAYMELSSLRSEDTAVYYCAXXXXXXXXXXXXXXXXXXXXWGQGTLVTVSS

SEQ ID NO: 33 - protein, 132 aa, Artificial Sequence
  VARIANT 26, 27, 28, 29, 30, 31, 32, 33, 34, 35, 36, 37, 51, 52,53, 54, 55, 56, 57, 58, 59, 60, 61, 62, 63, 64, 65, 66, 67,68, 69, 70, 101, 102, 103, 104, 105, 106, 107, 108, 109,110, 111, 112, 113, 114, 115, 116, 117, 118, 119, 120, 121: Xaa = Any Amino Acid
  HEAVY CHAIN FRAMEWORK REGION
QVQLVQSGAEVKKPGASVKVSCKASXXXXXXXXXXXXWVRQAPGQGLEWMXXXXXXXXXXXXXXXXXXXXRVTITADTSTSTAYMELSSLRSEDTAVYYCXXXXXXXXXXXXXXXXXXXXXWGQGTLVTVSS

SEQ ID NO: 34 - protein, 132 aa, Artificial Sequence
  VARIANT 31, 32, 33, 34, 35, 36, 37, 52, 53, 54, 55, 56, 57, 58,59, 60, 61, 62, 63, 64, 65, 66, 67, 68, 69, 70, 103, 104, 105,106, 107, 108, 109, 110, 111, 112, 113, 114, 115, 116, 117,118, 119, 120, 121: Xaa = Any Amino Acid
  HEAVY CHAIN FRAMEWORK REGION
QVQLQESGPGLVKPSQTLSLTCTVSGGSVSXXXXXXXWIRQPPGKGLEWIGXXXXXXXXXXXXXXXXXXXRVTISVDTSKNQFSLKLSSVTAADTAVYYCARXXXXXXXXXXXXXXXXXXXWGQGTLVTVSS

SEQ ID NO: 35 - protein, 132 aa, Artificial Sequence
  VARIANT 26, 27, 28, 29, 30, 31, 32, 33, 34, 35, 36, 37, 51, 52,53, 54, 55, 56, 57, 58, 59, 60, 61, 62, 63, 64, 65, 66, 67,68, 69, 70, 103, 104, 105, 106, 107, 108, 109, 110, 111,112, 113, 114, 115, 116, 117, 118, 119, 120, 121: Xaa = Any Amino Acid
  HEAVY CHAIN FRAMEWORK REGION
QVQLQESGPGLVKPSQTLSLTCTVSXXXXXXXXXXXXWIRQPPGKGLEWIXXXXXXXXXXXXXXXXXXXXRVTISVDTSKNQFSLKLSSVTAADTAVYYCARXXXXXXXXXXXXXXXXXXXWGQGTLVTVSS

SEQ ID NO: 36 - protein, 132 aa, Artificial Sequence
  VARIANT 26, 27, 28, 29, 30, 31, 32, 33, 34, 35, 36, 37, 51, 52,53, 54, 55, 56, 57, 58, 59, 60, 61, 62, 63, 64, 65, 66, 67,68, 69, 70, 102, 103, 104, 105, 106, 107, 108, 109, 110,111, 112, 113, 114, 115, 116, 117, 118, 119, 120, 121: Xaa = Any Amino Acid
  HEAVY CHAIN FRAMEWORK REGION
QVQLQESGPGLVKPSQTLSLTCTVSXXXXXXXXXXXXWIRQPPGKGLEWIXXXXXXXXXXXXXXXXXXXXRVTISVDTSKNQFSLKLSSVTAADTAVYYCAXXXXXXXXXXXXXXXXXXXXWGQGTLVTVSS

SEQ ID NO: 37 - protein, 132 aa, Artificial Sequence
  VARIANT 26, 27, 28, 29, 30, 31, 32, 33, 34, 35, 36, 37, 51, 52,53, 54, 55, 56, 57, 58, 59, 60, 61, 62, 63, 64, 65, 66, 67,68, 69, 70, 101, 102, 103, 104, 105, 106, 107, 108, 109,110, 111, 112, 113, 114, 115, 116, 117, 118, 119, 120, 121: Xaa = Any Amino Acid
  HEAVY CHAIN FRAMEWORK REGION
QVQLQESGPGLVKPSQTLSLTCTVSXXXXXXXXXXXXWIRQPPGKGLEWIXXXXXXXXXXXXXXXXXXXXRVTISVDTSKNQFSLKLSSVTAADTAVYYCXXXXXXXXXXXXXXXXXXXXXWGQGTLVTVSS

SEQ ID NO: 38 - protein, 132 aa, Artificial Sequence
  VARIANT 31, 32, 33, 34, 35, 36, 37, 52, 53, 54, 55, 56, 57, 58,59, 60, 61, 62, 63, 64, 65, 66, 67, 68, 69, 70, 103, 104, 105,106, 107, 108, 109, 110, 111, 112, 113, 114, 115, 116, 117,118, 119, 120, 121: Xaa = Any Amino Acid
  HEAVY CHAIN FRAMEWORK REGION
EVQLVESGGGLVQPGGSLRLSCAASGFTFSXXXXXXXWVRQAPGKGLEWVSXXXXXXXXXXXXXXXXXXXRFTISRDNSKNTLYLQMNSLRAEDTAVYYCARXXXXXXXXXXXXXXXXXXXWGQGTLVTVSS

SEQ ID NO: 39 - protein, 132 aa, Artificial Sequence
  VARIANT 26, 27, 28, 29, 30, 31, 32, 33, 34, 35, 36, 37, 51, 52,53, 54, 55, 56, 57, 58, 59, 60, 61, 62, 63, 64, 65, 66, 67,68, 69, 70, 103, 104, 105, 106, 107, 108, 109, 110, 111,112, 113, 114, 115, 116, 117, 118, 119, 120, 121: Xaa = Any Amino Acid
  HEAVY CHAIN FRAMEWORK REGION
EVQLVESGGGLVQPGGSLRLSCAASXXXXXXXXXXXXWVRQAPGKGLEWVXXXXXXXXXXXXXXXXXXXXRFTISRDNSKNTLYLQMNSLRAEDTAVYYCARXXXXXXXXXXXXXXXXXXXWGQGTLVTVSS

SEQ ID NO: 40 - protein, 132 aa, Artificial Sequence
  VARIANT 26, 27, 28, 29, 30, 31, 32, 33, 34, 35, 36, 37, 51, 52,53, 54, 55, 56, 57, 58, 59, 60, 61, 62, 63, 64, 65, 66, 67,68, 69, 70, 102, 103, 104, 105, 106, 107, 108, 109, 110,111, 112, 113, 114, 115, 116, 117, 118, 119, 120, 121: Xaa = Any Amino Acid
  HEAVY CHAIN FRAMEWORK REGION
EVQLVESGGGLVQPGGSLRLSCAASXXXXXXXXXXXXWVRQAPGKGLEWVXXXXXXXXXXXXXXXXXXXXRFTISRDNSKNTLYLQMNSLRAEDTAVYYCAXXXXXXXXXXXXXXXXXXXXWGQGTLVTVSS

SEQ ID NO: 41 - protein, 132 aa, Artificial Sequence
  VARIANT 26, 27, 28, 29, 30, 31, 32, 33, 34, 35, 36, 37, 51, 52,53, 54, 55, 56, 57, 58, 59, 60, 61, 62, 63, 64, 65, 66, 67,68, 69, 70, 101, 102, 103, 104, 105, 106, 107, 108, 109,110, 111, 112, 113, 114, 115, 116, 117, 118, 119, 120, 121: Xaa = Any Amino Acid
  HEAVY CHAIN FRAMEWORK REGION
EVQLVESGGGLVQPGGSLRLSCAASXXXXXXXXXXXXWVRQAPGKGLEWVXXXXXXXXXXXXXXXXXXXXRFTISRDNSKNTLYLQMNSLRAEDTAVYYCXXXXXXXXXXXXXXXXXXXXXWGQGTLVTVSS

SEQ ID NO: 42 - protein, 132 aa, Artificial Sequence
  VARIANT 31, 32, 33, 34, 35, 36, 37, 52, 53, 54, 55, 56, 57, 58,59, 60, 61, 62, 63, 64, 65, 66, 67, 68, 69, 70, 103, 104, 105,106, 107, 108, 109, 110, 111, 112, 113, 114, 115, 116, 117,118, 119, 120, 121: Xaa = Any Amino Acid
  HEAVY CHAIN FRAMEWORK REGION
EVQLVESGGGLVQPGGSLRLSCAASGFNIKXXXXXXXWVRQAPGKGLEWVSXXXXXXXXXXXXXXXXXXXRFTISADTSKNTAYLQMNSLRAEDTAVYYCSRXXXXXXXXXXXXXXXXXXXWGQGTLVTVSS

SEQ ID NO: 43 - protein, 132 aa, Artificial Sequence
  VARIANT 26, 27, 28, 29, 30, 31, 32, 33, 34, 35, 36, 37, 51, 52,53, 54, 55, 56, 57, 58, 59, 60, 61, 62, 63, 64, 65, 66, 67,68, 69, 70, 103, 104, 105, 106, 107, 108, 109, 110, 111,112, 113, 114, 115, 116, 117, 118, 119, 120, 121: Xaa = Any Amino Acid
  HEAVY CHAIN FRAMEWORK REGION
EVQLVESGGGLVQPGGSLRLSCAASXXXXXXXXXXXXWVRQAPGKGLEWVXXXXXXXXXXXXXXXXXXXXRFTISADTSKNTAYLQMNSLRAEDTAVYYCSRXXXXXXXXXXXXXXXXXXXWGQGTLVTVSS

SEQ ID NO: 44 - protein, 132 aa, Artificial Sequence
  VARIANT 26, 27, 28, 29, 30, 31, 32, 33, 34, 35, 36, 37, 51, 52,53, 54, 55, 56, 57, 58, 59, 60, 61, 62, 63, 64, 65, 66, 67,68, 69, 70, 102, 103, 104, 105, 106, 107, 108, 109, 110,111, 112, 113, 114, 115, 116, 117, 118, 119, 120, 121: Xaa = Any Amino Acid
  HEAVY CHAIN FRAMEWORK REGION
EVQLVESGGGLVQPGGSLRLSCAASXXXXXXXXXXXXWVRQAPGKGLEWVXXXXXXXXXXXXXXXXXXXXRFTISADTSKNTAYLQMNSLRAEDTAVYYCSXXXXXXXXXXXXXXXXXXXXWGQGTLVTVSS

SEQ ID NO: 45 - protein, 132 aa, Artificial Sequence
  VARIANT 31, 32, 33, 34, 35, 36, 37, 52, 53, 54, 55, 56, 57, 58,59, 60, 61, 62, 63, 64, 65, 66, 67, 68, 69, 70, 103, 104, 105,106, 107, 108, 109, 110, 111, 112, 113, 114, 115, 116, 117,118, 119, 120, 121: Xaa = Any Amino Acid
  HEAVY CHAIN FRAMEWORK REGION
EVQLVESGGGLVQPGGSLRLSCAASGFNIKXXXXXXXWVRQAPGKGLEWVSXXXXXXXXXXXXXXXXXXXRFTISADTSKNTAYLQMNSLRAEDTAVYYCARXXXXXXXXXXXXXXXXXXXWGQGTLVTVSS

SEQ ID NO: 46 - protein, 132 aa, Artificial Sequence
  VARIANT 26, 27, 28, 29, 30, 31, 32, 33, 34, 35, 36, 37, 51, 52,53, 54, 55, 56, 57, 58, 59, 60, 61, 62, 63, 64, 65, 66, 67,68, 69, 70, 103, 104, 105, 106, 107, 108, 109, 110, 111,112, 113, 114, 115, 116, 117, 118, 119, 120, 121: Xaa = Any Amino Acid
  HEAVY CHAIN FRAMEWORK REGION
EVQLVESGGGLVQPGGSLRLSCAASXXXXXXXXXXXXWVRQAPGKGLEWVXXXXXXXXXXXXXXXXXXXXRFTISADTSKNTAYLQMNSLRAEDTAVYYCARXXXXXXXXXXXXXXXXXXXWGQGTLVTVSS

SEQ ID NO: 47 - protein, 132 aa, Artificial Sequence
  VARIANT 26, 27, 28, 29, 30, 31, 32, 33, 34, 35, 36, 37, 51, 52,53, 54, 55, 56, 57, 58, 59, 60, 61, 62, 63, 64, 65, 66, 67,68, 69, 70, 102, 103, 104, 105, 106, 107, 108, 109, 110,111, 112, 113, 114, 115, 116, 117, 118, 119, 120, 121: Xaa = Any Amino Acid
  HEAVY CHAIN FRAMEWORK REGION
EVQLVESGGGLVQPGGSLRLSCAASXXXXXXXXXXXXWVRQAPGKGLEWVXXXXXXXXXXXXXXXXXXXXRFTISADTSKNTAYLQMNSLRAEDTAVYYCAXXXXXXXXXXXXXXXXXXXXWGQGTLVTVSS

SEQ ID NO: 48 - protein, 132 aa, Artificial Sequence
  VARIANT 26, 27, 28, 29, 30, 31, 32, 33, 34, 35, 36, 37, 51, 52,53, 54, 55, 56, 57, 58, 59, 60, 61, 62, 63, 64, 65, 66, 67,68, 69, 70, 101, 102, 103, 104, 105, 106, 107, 108, 109,110, 111, 112, 113, 114, 115, 116, 117, 118, 119, 120, 121: Xaa = Any Amino Acid
  HEAVY CHAIN FRAMEWORK REGION
EVQLVESGGGLVQPGGSLRLSCAASXXXXXXXXXXXXWVRQAPGKGLEWVXXXXXXXXXXXXXXXXXXXXRFTISADTSKNTAYLQMNSLRAEDTAVYYCXXXXXXXXXXXXXXXXXXXXXWGQGTLVTVSS

SEQ ID NO: 49 - protein, 82 aa, Artificial Sequence
  VARIANT 24, 40: Xaa = Any Amino Acid
  LIGHT CHAIN FRAMEWORK REGION
DIQMTQSPSSLSASVGDRVTITCXWYQQKPGKAPKLLIYXGVPSRFSGSGSGTDFTLTISSLQPEDFATYYCFGQGTKVEIK

SEQ ID NO: 50 - protein, 80 aa, Artificial Sequence
  LIGHT CHAIN FRAMEWORK REGION
DIVMTQSPLSLPVTPGEPASISCWYLQKPGQSPQLLIYGVPDRFSGSGSGTDFTLKISRVEAEDVGVYYCFGQGTKVEIK

SEQ ID NO: 51 - protein, 80 aa, Artificial Sequence
  LIGHT CHAIN FRAMEWORK REGION
EIVLTQSPGTLSLSPGERATLSCWYQQKPGQAPRLLIYGIPDRFSGSGSGTDFTLTISRLEPEDFAVYYCFGQGTKVEIK

SEQ ID NO: 52 - protein, 80 aa, Artificial Sequence
  LIGHT CHAIN FRAMEWORK REGION
DIVMTQSPDSLAVSLGERATINCWYQQKPGQPPKLLIYGVPDRFSGSGSGTDFTLTISSLQAEDVAVYYCFGQGTKVEIK

SEQ ID NO: 53 - protein, 23 aa, Artificial Sequence
  LIGHT CHAIN FRAMEWORK REGION
DIQMTQSPSSLSASVGDRVTITC

SEQ ID NO: 54 - protein, 15 aa, Artificial Sequence
  LIGHT CHAIN FRAMEWORK REGION
WYQQKPGKAPKLLIY

SEQ ID NO: 55 - protein, 32 aa, Artificial Sequence
  LIGHT CHAIN FRAMEWORK REGION
GVPSRFSGSRSGTDFTLTISSLQPEDFATYYC

SEQ ID NO: 56 - protein, 10 aa, Artificial Sequence
  LIGHT CHAIN FRAMEWORK REGION
FGQGTKVEIK

SEQ ID NO: 57 - protein, 25 aa, Artificial Sequence [sequence omitted]

SEQ ID NO: 58 - protein, 13 aa, Artificial Sequence
  LIGHT CHAIN FRAMEWORK REGION
WVRQAPGKGLEWV

SEQ ID NO: 59 - protein, 30 aa, Artificial Sequence
  LIGHT CHAIN FRAMEWORK REGION
RFTISADTSKNTAYLQMNSLRAEDTAVYYC

SEQ ID NO: 60 - protein, 11 aa, Artificial Sequence
  LIGHT CHAIN FRAMEWORK REGION
WGQGTLVTVSS

(19) United States
(12) Patent Application Publication
(10) Pub. No.: US 2010/0136030 A1
(43) Pub. Date: Jun. 3, 2010

(54) ANTAGONIST OX40 ANTIBODIES AND THEIR USE IN THE TREATMENT OF INFLAMMATORY AND AUTOIMMUNE DISEASES

(76) Inventors: Lamhamedi-Cherradi Salah-Eddine, Pearland, TX (US); Yao Zhengbin, Berwyn, PA (US); Singh Sanjaya, Sandy Hook, CT (US)

Correspondence Address: JONES DAY, 222 EAST 41ST ST, NEW YORK, NY, 10017, US

(21) Appl. No.: 12/526,550

(22) PCT Filed: Feb. 26, 2008
(86) PCT No.: PCT/US08/02498

§ 371 Date: Feb. 3, 2010

Related U.S. Application Data
(60) Provisional application No. 60/903,693, filed on Feb. 27, 2007.

(51) Int. Cl. A61K 39/395; G01N 33/53; C12P 21/00; C12N 5/12; C12N 5/10; C12N 1/21; C12N 1/19; C12N 15/63; C07K 16/28; C12N 15/13; A61P 3/10; A61P 1/04; A61P 37/06; A61P 29/00; A61P 25/28; A61P 17/00; A61P 7/06; A61P 1/16; A61P 11/06; A61P 11/02

(52) U.S. Cl. 424/173.1; 435/7.1; 435/7.92; 435/7.94; 435/69.6; 435/325; 435/348; 435/252.3; 435/254.2; 435/320.1; 530/387.1; 530/387.3; 530/388.22; 536/23.53

(57) ABSTRACT

The present invention relates to antagonist antibodies directed against human OX40 receptor (CD134) and fragments thereof, including the amino acid sequences of antagonist antibodies and the nucleic acids that encode the antibodies. Also included in the present invention are antigen binding regions (CDRs) derived from the light and/or heavy chain variable regions of said antibodies. Another aspect of the present invention is the use of anti-OX40 antagonist antibodies in the treatment of inflammatory and autoimmune diseases. The present invention also relates to humanized sequences of an antagonist antibody A10 and epitope mapping of the binding site of the antibody.

This application is a national stage application of International Application No. PCT/US2008/002498, filed on Feb. 26, 2008, which claims priority to U.S. Provisional Patent Application No. 60/903,693, filed on Feb. 27, 2007, both of which are incorporated herein in their entireties.

### 1. FIELD OF INVENTION

Provided herein are antibodies that immunospecifically bind to a human OX40 polypeptide, a OX40 polypeptide fragment or other OX40 epitope. The invention is also directed to humanized antibodies that immunospecifically bind to a human OX40 polypeptide, OX40 polypeptide fragment or OX40 epitope. Also provided are isolated nucleic acids encoding antibodies that immunospecifically bind to an OX40 polypeptide, OX40 polypeptide fragment, or OX40 epitope. The invention further provides vectors and host cells comprising nucleic acids encoding antibodies that immunospecifically bind to a human OX40 polypeptide, OX40 polypeptide fragment, or OX40 epitope, as well as methods of making the antibodies. Also provided are methods of using the anti-OX40 antibodies provided herein to inhibit OX40 biological activity and/or to treat or prevent an OX40-mediated disease.

### 2. BACKGROUND OF THE INVENTION

Our immune system defends our body against foreign pathogens, a function that requires a balance of immune activation against foreign agents and tolerance to self tissues. Acute infections caused by, e.g., a viral pathogen, induce two types of long-term memory: humoral immunity, in which B-lymphocytes (B-cells) produce antibodies to prevent infection by these foreign pathogens; and cellular immunity, in which T-lymphocytes (T-cells) activated by specific antigens kill the infected cells and also produce cytokines. T-cells function primarily in cell-mediated immunity, and comprise approximately 70% of lymphocytes. The majority of T-cells are CD4+ “helper” T-cells, and are involved primarily in the activation of B-cells and macrophages. CD8+ T-cells, or cytotoxic T-lymphocytes (CTLs), are involved in cell-mediated cytotoxic reactions and comprise approximately 35% of the T-cell population. T-cells can be divided into three distinct populations: naïve, effector and memory cells. Naïve T-cells are activated and become effector cells in the presence of antigen. Major histocompatibility complex class I (MHC Class I) molecules play a primary role in this effector response by “presenting” antigens of invading pathogens to T-cell receptors, which in turn recognize the pathogens and attack them.

Many receptor-ligand interactions are involved in the induction, establishment, and modulation of immune responses directed against foreign antigens. At least two signals are necessary to activate a CD4⁺ or CD8⁺ T-cell response to an antigen. The first signal is delivered through the T-cell receptor (TCR) by an antigen bound to a major histocompatibility (MHC) class I or II molecule on the surface of an antigen-presenting cell (APC). The second signal involves the binding of a ligand present on the surface of the APC to a second receptor molecule on the surface of the T-cell. This second signal is termed costimulation, and the APC ligand is often referred to as a costimulatory molecule (Lenschow, et al., Annu Rev Immunol 14:233 (1996)). Costimulatory signaling molecules include immunoglobulin superfamily members, tumor necrosis factor receptor (TNFR) superfamily members, and cytokine receptors (see review Croft, Cytokine Growth Factor Rev 14:265 (2003); Kroczek, et al., J Allergy Clin Immunol 116:906 (2005)). In combination, the two signals activate the T-cell, which in turn secretes cytokines and proliferates.

One example of a costimulatory molecule is the OX40 receptor (CD134), a member of the TNFR superfamily, which is membrane-bound and is expressed primarily on activated CD4⁺ T-cells (i.e., at sites of inflammation) (Paterson, et al., Mol Immunol 24:1281 (1987)). Its ligand (OX40L) is a type-II membrane protein belonging to the TNF family and is expressed on antigen-presenting cells, such as activated B cells, dendritic cells, and endothelial cells (Stuber, et al., Immunity 2:507 (1995); Weinberg, et al., J Immunol 162:1818 (1999); Nohara, et al., J Immunol 166:2108 (2001); Malmstrom, et al., J Immunol 166:6972 (2001)). Signaling through the OX40 receptor (hereinafter “OX40”) is costimulatory to effector T-cells and causes proliferation of T-cells (Weinberg, J Immunol 152:4712 (1994); see review Watts, Annu Rev Immunol 23:23 (2005)). Studies of OX40 suggest that its major role is to dictate the number of effector T-cells that accumulate in primary immune responses, and consequently to govern the number of memory T-cells that subsequently develop and survive see review Croft, Cytokine Growth Factor Rev 14: 265 (2003)).

A number of in vitro studies have been shown that OX40 provides a costimulatory signal resulting, in enhanced T-cell proliferation and cytokine production (Baum P R et al., EMBO J, 1994: Akiba H et al. Biochem. Biophysic Res, 1998). It has been suggested that in certain circumstances the OX40/OX40L interaction may play a preferential role in the development of Th2 cells (Ohshima, et al., Blood 92:3338 (1998); Flynn, et al., J Exp Med 188:297 (199. When immune activation is excessive or uncontrolled, pathological allergy, asthma, inflammation, autoimmune and other related diseases may occur. In such instances, activation and differentiation of T-cells play an important role. Because OX40 functions to enhance immune responses, it may exacerbate autoimmune and inflammatory diseases. The interaction of OX40-OX40L has been implicated in the pathogenesis of several disease models. A large body of evidence suggests that the OX40-OX40L interaction plays an important role in allergy, asthma, and diseases associated with autoimmunity and inflammation, which include multiple sclerosis, rheumatoid arthritis, inflammatory bowel disease, graft-versus-host disease, experimental autoimmune encephalomyelitis (EAE), experimental leishmaniasis, collagen-induced arthritis, colitis (such as ulcerative colitis), contact hypersensitivity reactions, diabetes, Crohn's Disease, and Grave's Disease (Arestides, et al., Eur J Immunol 32:2874 (2002); Jember, et al., J Exp Med 193:387 (2001); Kroczek, et al., J Allergy Clin Immunol 116:906 (2005); Pakala, et al., Eur J Immunol 34:3039 (2004); Xiaoyan, et al., Clin Exp Immunol 143:110 (2006); Weinberg, et al., J Immunol 152:4712 (1994); Weinberg, et al., Nat Med 2:183 (1996); Malstrom, et al., J Immunol 166:6972 (2001); Higgins, et al., J Immunol 162:486 (1999); Akiba, et al., J Exp Med 191:375 (2000); Stuber, et al., Gastroenterology 115:1205 (1998); Tsukada, et al., Blood 95:2434 (2000); Yoshioka, et al., Eur J Immunol 30:2815 (2000); Stuber, et al., Eur Clin Invest 30:594 (2000); Bossowski, et al., J Pediatr Endocrinol Metab 18:1365 (2005); see review Watts, Annu Rev Immunol 23:23 (2005)).

Current therapies for autoimmune and inflammatory disease include long-term administration of steroids alone or in combination with cytotoxic drugs and/or biologics. Antibodies against TNF-α are currently being prescribed for a number of diseases, including rheumatoid arthritis, Crohn's disease, and inflammatory bowel disease. In addition, anew experimental therapy in clinical evaluation is a fusion protein CTLA4-Ig (cytotoxic T lymphocyte associated antigen-4 in a soluble form) for rheumatoid arthritis. Despite these alternatives, most immunosuppressive therapies cause serious side effects, including the patient's immunocompromised state. Ideally, treatment of T-cell-mediated diseases would target the disease causing [antigen-specific] cells, but spare the rest of the T-cell repertoire (Weinberg, Trends Immunol 23:102 (2002)). Therefore, there is a strong need for alternative therapies for autoimmune and inflammatory diseases.

In addition to these methods, many agonist anti-OX40 antibodies have been developed that stimulate the receptor to increase the T-cell population. The first published anti-OX40 monoclonal antibody (mAb), MRC OX-40, was a mouse antibody that identified the receptor as a cell surface antigen on activated rat CD4⁺ T-cells (Paterson, et al., Mol Immunol 24:1281 (1987)). The antibody had a modest effect in stimulating T-cell proliferation assays. Since then, many agonist anti-OX40 mAbs have been produced and used to boost the immune response (Weatherill, et al., Cell Immunol 209:63 (2001); Banal-Pakala, et al., Nat Med 7:907 (2001); De Smedt, et al., J Immunol 168:661 (2002); Pan, et al., Mol Ther 6:528 (2002); Curti, et al., Blood 101:568 (2003); Nakae, et al., Proc Natl Acad Sci USA 100:5986 (2003); Lustgarten, et al., Eur J Immunol 34:752 (2004); So, et al., J Immunol 172:4292 (2004); Koga, et al., Cancer Sci 95:411 (2004); Lanthrop, et al., J Immunol 172:6735 (2004); Polymenidou, et al., Proc Natl Acad Sci USA 101 Suppl 2:14670 (2004); Lustgarten, et al., J Immunol 173:4510 (2004); Valzasina, et al., Blood 105:2845 (2005); Cuadros, et al., Int J Cancer 116:934 (2005); Sharma, et al., Exp Gerontol 41:78 (2006)). However, in the previously mentioned autoimmune and inflammatory diseases, stimulating the T-cell population is not the desired result.

One therapy is to block OX40-OX40L signaling is through the use of anti-OX40L antibodies. A number of mAbs targeting the OX40L has been generated and tested to elucidate the OX40 signaling pathway, its effects on other pathways, and the OX40 role in various diseases (See, e.g., Blazar, et al., Blood 101:3741 (2003); Ukyo, et al., Immunology 109:226 (2003); Wang, et al., Tissue Antigens 64:566 (2004); Chou, et al., J Immunol 174:436 (2005)). Other methods of inhibiting the OX40 signal include the use of anti-OX40 immunotoxins, OX40-IgG fusion proteins, and OX40 liposomes (Weinberg, et al., Nat Med 2:193 (1996); Higgins, et al., J Immunol 162:486 (1999); Satake, et al., Biochem Biophys Res Commun 270:1041 (2000); Taylor, et al., J Leukoc Biol 72:522 (2002); Boot, et al., Arthritis Res Ther 7:R604 (2005)).

An alternative to targeting the ligand is to develop a therapy directed against OX40 receptor (CD134). There have been attempts to generate an antagonist anti-OX40 antibody. Stuber and colleagues used a polyclonal rabbit anti-mouse OX40 antibody to inhibit the interaction between OX40 and OX40L (J Exp Med 183:979 (1996)). Imura, et al. produced anti-mouse OX40 antibodies, 131 and 315, that inhibited adhesion of CD4⁺ T-cells to vascular endothelial cells and the proliferation of T-cells, processes mediated by the OX40 pathway. The anti-human OX40 antibody disclosed by Weinberg exhibited the ability to deplete activated CD4⁺ T-cells: however, it relied on the antibody's conjugation to anoxic molecule, such as Ricin-A chain (U.S. Pat. No. 5,759,546). Other anti-OX40 antibodies disclosed had no functional activity, such as commercially available L106.

Thus, there is a need for a true functional antagonist antibody targeting human OX40 with the capability to interfere with the OX40 pathway. Such an antibody would have the potential to treat a plethora of diseases that currently have a large unmet need. The present invention solves this unmet medical need.

### 3. SUMMARY OF THE INVENTION

In one aspect, the present invention relates to antagonist antibodies directed against human OX40 receptor (CD134) and fragments thereof. Another embodiment includes the amino acid sequences of antagonist antibodies and the nucleic acids that encode the antibodies.

Also included in the present invention are antigen binding regions (CDRs) derived from the light and/or heavy chain variable regions of said antibodies. The antibodies of the invention may be a recombinant antibody. The antibodies of the invention may be monoclonal, and a monoclonal antibody may be a human antibody, a chimeric antibody, or a humanized antibody.

The present invention includes an antibody that comprises a heavy chain variable region (VH) comprising a VH CDR1 having the amino acid sequence of SEQ ID NO:17; a VH CDR2 having the amino acid sequence of SEQ ID NO:18; and/or a VH CDR3 having the amino acid sequence of SEQ ID NO:19; and/or a light chain variable region (VL) comprising a VL CDR1 having the amino acid sequence of SEQ ID NO:12, 15 or 61; a VL CDR2 having the amino acid sequence of SEQ ID NO:13; and/or a VL CDR3 having the amino acid sequence of SEQ ID NO:14 or 16. In an embodiment, the antibody comprises two VH CDRs having the amino acid sequence selected from SEQ ID NO.:17, 18, or 19 and/or two VL CDRs having the amino acid sequence selected from SEQ ID NO: 12, 13, 14, 15, 16 or 61.

The present invention includes an antibody that comprises a heavy chain variable region (VH) comprising a VH CDR1 having the amino acid sequence of SEQ ID NO:17; a VH CDR2 having the amino acid sequence of SEQ ID NO:18; and a VH CDR3 having the amino acid sequence of SEQ ID NO:19; and/or a light chain variable region (VL) comprising a VL CDR1 having the amino acid sequence of SEQ ID NO:12; a VL CDR2 having the amino acid sequence of SEQ ID NO:13; and a VL CDR3 having the amino acid sequence of SEQ ID NO:14.

The present invention includes an antibody that comprises a heavy chain variable region (VH) comprising a VH CDR1 having the amino acid sequence of SEQ ID NO:17; a VH CDR2 having the amino acid sequence of SEQ ID NO:18; and a VH CDR3 having the amino acid sequence of SEQ ID NO:19; and/or a light chain variable region (VL) having the amino acid sequence of a VL CDR1 having the amino acid sequence of SEQ ID NO:12; a VL CDR2 having the amino acid sequence of SEQ ID NO:13; and a VL CDR3 having the amino acid sequence of SEQ ID NO:16.

The present invention includes an antibody that comprises a heavy chain variable region (VH) comprising a VH CDR1 having the amino acid sequence of SEQ ID NO:17 a VH CDR2 having the amino acid sequence of SEQ ID NO:18; and a VH CDR3 having the amino acid sequence of SEQ ID NO:19; and/or a light chain variable region (VL) comprising: a VL CDR1 having the amino acid sequence of SEQ ID NO:15; a VL CDR2 having the amino acid sequence of SEQ ID NO:13; and a VL CDR3 having the amino acid sequence of SEQ ID NO:14.

The present invention includes an antibody that comprises a heavy chain variable region (VH) comprising a VH CDR1 having the amino acid sequence of SEQ ID NO:17; a VH CDR2 having the amino acid sequence of SEQ ID NO:18; and a VH CDR3 having the amino acid sequence of SEQ ID NO:19; and/or a light chain variable region (VL) comprising a VL CDR1 having the amino acid sequence of SEQ ID NO:15; a VL CDR2 having the amino acid sequence of SEQ ID NO:13; and a VL CDR3 having the amino acid sequence of SEQ ID NO:16.

The present invention includes an antibody that comprises a heavy chain variable region (VH) having the amino acid sequence depicted in any one of SEQ ID NO:10 or 11 and/or a light chain variable region (VL) having the amino acid sequence depicted in any one of SEQ ID NO:7, 8, or 9.

The present invention includes an antibody that comprises a heavy chan variable region (VH) encoded by any one of the nucleic acid sequence of SEQ ID NO: 25, 26, or 27; and/or a light chain variable region (VL) encoded by any one the nucleic acid sequence of SEQ ID NO: 20, 21, 22, 23, or 24.

The present invention includes an antibody that comprises a VH having the amino acid sequence depicted in SEQ ID NO:10 and a VL having the amino acid sequence depicted in any one of SEQ ID NO: 7 or 8: a VH having the amino acid sequence depicted in SEQ ID NO:11 and a VL depicted in SEQ ID NO: 9; a VH encoded by the nucleic acid sequence of SEQ ID NO:25 and a VL encoded by the nucleic acid sequence of SEQ ID NO: 20, or 21; a VH encoded by the nucleic acid sequence of SEQ ID NO:26 and a VL encoded by the nucleic acid sequence of SEQ ID NO: 22, 23, or 24; or a VH encoded by the nucleic acid sequence of SEQ ID NO:27 and a VL encoded by the nucleic acid sequence of SEQ ID NO: 22, 23, or 24.

The present invention includes an antibody wherein the VH comprises the amino acid sequence depicted in SEQ ID NO:10 and the VL comprises the amino acid sequence depicted in SEQ ID NO: 7.

The present invention includes an antibody wherein the VH comprises the amino acid sequence depicted in SEQ ID NO:10 and the VL comprises the amino acid sequence depicted in SEQ ID NO: 8.

The present invention includes an antibody wherein the VH comprises the amino acid sequence depicted in SEQ ID NO:11 and the VL comprises the amino acid sequence depicted in SEQ ID NO: 9.

The present invention includes an antibody wherein the VH is encoded by a nucleotide sequence that hybridizes under stringent conditions to the complement of a nucleotide sequence that encodes the amino acid sequence of SEQ ID NO:10,11,17,18, or 19; and/or a VL encoded by a nucleotide sequence that hybridizes under stringent conditions to the complement of a nucleotide sequence that encodes the amino acid sequence of SEQ ID NO:7, 8, 9, 12, 13, 14, 15, 16 or 61.

The present invention includes an antibody wherein the VH is encoded by a nucleotide sequence that hybridizes under stringent conditions to the complement of a nucleotide sequence as depicted in any one of SEQ ID NO: 25, 26, or 27; and/or a VL encoded by a nucleotide sequence that hybridizes under stringent conditions to the complement of a nucleotide sequence as depicted in any one of SEQ ID NO:20, 21, 22, 23, or 24.

The present invention includes an isolated antagonist antibody that specifically binds to a human OX40 epitope with a dissociation constant between about 1×10⁻¹² to about 1×10⁻¹¹M, 1×10⁻¹¹ to about 1×10⁻¹⁰M, 1×10⁻¹⁰ to about 1×10⁻⁹M, 1×10⁻⁹ to about 1×10⁻⁸M, 1×10⁻⁸ to about 1×10⁻⁷M.

The present invention includes a VL sequence having at least 95% sequence identity to that set forth in SEQ ID NO: 7, and a VH sequence at least 95% sequence identity to that set forth in SEQ ID NO: 10.

The present invention includes a VL sequence having at least 95% sequence identity to that set forth in SEQ ID NO: 8, and a VH sequence at least 95% sequence identity to that set forth in SEQ ID NO: 10.

The present invention includes a VL sequence having at least 95% sequence identity to that set forth in SEQ ID NO: 9, and a VH sequence at least 95% sequence identity to that set forth in SEQ ID NO: 11.

The present invention also includes an antibody molecule comprising a heavy chain variable region comprising SEQ ID NO: 17 (CDR-H1), SEQ ID NO: 18 (CDR-H2) and SEQ ID NO: 19 (CDR-H3) and/or a light chain variable region comprising SEQ ID NO: 12, SEQ ID NO: 15 or SEQ ID NO:61 (CDR-L1); SEQ ID NO: 13 (CDR-L2); and SEQ ID NO: 14 or SEQ ID NO: 16 (CDR-L3).

The present invention includes human antigen-binding antibody fragments of the antibodies of the present invention including, but not limited to, Fab, Fab′ and F(ab′)₂, Fd, single-chain Fvs (scFv), single-chain antibodies, disulfide-linked Fvs (sdFv), diabodies, triabodies, or minibodies. The invention also includes single-domain antibodies comprising either a VL or VH domain.

The present invention includes humanized sequences of monoclonal antibody A10(TH)hu336F and variants thereof. Nucleic acids encoding these variants include SEQ ID NOs 20-27. A10(TH)hu336F comprises a light chain variable region comprising SEQ ID NO: 9 and a heavy chain variable region comprising SEQ ID NO: 11. The variable heavy chain region may further comprise at least one domain from CH1, CH2 and CH3 domains of a constant region. The heavy chain constant region may be an IgG antibody, wherein the IgG antibody is an IgG1 antibody, an IgG2 antibody, an IgG3 antibody, or an IgG4 antibody.

Antibodies of the invention can comprise any suitable framework variable domain sequence, provided binding activity to OX40 is substantially retained. For example, in some embodiments, antibodies of the invention comprise a human subgroup III heavy chain framework consensus sequence. In one embodiment of these antibodies, the framework consensus sequence comprises substitution at position 71, 73 and/or 78. In some embodiments of these antibodies, position 71 is A, 73 is T and/or 78 is A. In one embodiment, these antibodies comprise heavy chain variable domain framework sequences of huMAb4D5-8 (HERCEPTIN® Genentech, Inc., South San Francisco. CA, USA) (also referred to in U.S. Pat. Nos. 6,407,213 & 5,821,337, and Lee et al., J. Mol. Biol. (2004), 340(5):1073-93). In one embodiment, these antibodies further comprise a human κI light chain framework consensus sequence. In one embodiment, the antibodies comprise a combination of IGHV1-46 of the subgroup V1 (Gene Bank Accession number X92343) and germ line IGHJ4 (Gene Bank Accession Number J00256) shown in FIG. 12B (IGHV1-46 sequence). In one embodiment, the framework sequence comprises substitution at position 69. In some embodiments, the position 69 is L. The human template chosen for the V_{L} chain was a combination of IGKV4-1 (Gene Bank Accession Number Z00023) and Germ line J template IGHJ1 (Gene Bank Accession Number J00242) shown in FIG. 12A (IGKV4-1 sequence).

In one embodiment, an antibody of the invention comprises a heavy chain variable domain, wherein the framework sequence comprises the sequence of SEQ ID NOS: 30, 31, 32, 33, 34, 35, 36, 37, 38, 39, 40, 41, 42, 43, 44, 45, 46, 47 and/or 48. In one embodiment, an antibody of the invention comprises a light chain variable domain, wherein the framework sequence comprise the sequence of SEQ ID NOS: 49, 50, 51, and/or 52.

In one embodiment, an antibody of the invention comprises a heavy chain variable domain, wherein the framework sequence comprises the sequence of SEQ ID NOS: 57, 58, 59 and/or 60. In one embodiment, an antibody of the invention comprises a light chain variable domain, wherein the framework sequence comprise the sequence of SEQ ID NOS: 53, 54, 55, and/or 60.

In one embodiment, an antibody of the invention comprises a heavy chain variable domain, wherein the framework sequence comprises the sequence of SEQ ID NOS: 30, 31, 32, 33, 34, 35, 36, 37, 38, 39, 40, 41, 42, 43, 44, 45, 46, 47 and/or 48, and HVR H1, H2 and H3 sequences are SEQ ID NOS:17, 18, and/or 19, respectively. In one embodiment, an antibody of the invention comprises a light chain variable domain, wherein the framework sequence comprise the sequence of SEQ ID NOS: 49, 50, 51 and/or 52, and HVR L1, L2 and L3 sequences are SEQ ID NOS: 12, 13 and/or 16, respectively.

In one embodiment, an antibody of the invention comprises a heavy chain variable domain, wherein the framework sequence comprises the sequence of SEQ ID NOS: 57, 58, 59 and/or 60, and HVR H1, H2 and H3 sequences are SEQ ID NOS: 17, 18, and/or 19, respectively. In one embodiment, an antibody of the invention comprises a light chain variable domain, wherein the framework sequence comprise the sequence of SEQ ID NOS: 53, 54, 55 and/or 56, and HVR L1, L2 and L3 sequences are SEQ ID NOS: 12, 13 and/or 16, respectively.

The present invention includes an anti-OX40 antibody further comprising a detectable tag. The present invention further includes a method of detecting OX40 in vivo or in a sample. Such method comprises contacting the OX40 antibody with a subject or with a sample obtained from the subject.

The present invention includes an antibody further comprising a label.

The present invention includes the anti-OX40 antibodies described above further comprising a cytotoxin or immunotoxin and their use in treating the diseases or conditions described below.

The present invention also includes antibodies that bind the same epitope as antibody A10(TH)hu336F.

The present invention includes an isolated nucleic acid molecule which comprises a nucleotide sequence that encodes an antibody of the present invention.

The present invention includes a vector which comprises the nucleic acid molecule of the present invention.

The present invention includes a host cell which comprises the vector of the present invention.

The present invention includes a hybridoma that produces an antibody of the present invention.

The present invention includes an isolated nucleic acid molecule encoding an antibody of the present invention wherein the nucleotide sequence comprises any one of SEQ ID NOS: 20, 21, 22, 23, or 24.

The present invention includes an insolated nucleic acid molecule encoding an antibody of the present invention comprising the amino acid sequence of SEQ ID NO: 7, 8, 9, 10, 11, 12, 13, 14, 15, 16, 17, 18 or 19.

The present invention includes an isolated nucleic acid molecule comprising a nucleic acid sequence that encodes a heavy chain variable region (VH) amino acid sequence set forth in any one of SEQ ID NO: 10, 11, 17, 18 or 19, or encoded by the nucleic acid sequence of SEQ ID NO: 25, 26, or 27, and/or a light chain variable region (VL) amino acid sequence set forth in any one of SEQ ID NO: 7, 8, 9, 12, 13, 14, 15, or 16, or encoded by the nucleic acid sequence of SEQ ID NO: 20, 21, 22, 23, or 24.

The present invention includes a composition comprising the antibodies according to the claimed invention in combination with a physiologically acceptable carrier, diluents, excipient, or stabilizer.

The present invention includes a method for producing the antibodies of the claimed invention comprising culturing the cell of the present invention under conditions suitable for the production of the antibody, and isolation of the antibody.

Another aspect of the present invention is the use of anti-OX40 antagonist antibodies in the treatment of inflammatory and autoimmune diseases.

The present invention includes a method for preventing a disorder by inhibiting IgE antibody production in a patient, comprising administering to the patient an effective mount of an antibody according to the present invention. The disorder includes, but is not limited to asthma (such as allergic asthma), allergic rhinitis, atopic dermatitis, transplant rejection, and atherosclerosis.

The present invention includes a method for inhibiting IgE antibody production in a patient, which comprises administrating to the patient an antagonist anti-OX40 antibody according to the claimed invention. The inhibition of IgE antibody production may prevent bronchial asthma, allergic rhinitis, allergic dermatitis, anaphylaxis, uticaria, and atopic dermatitis.

The present invention includes a method of treating an OX-40-mediated disorder in a patient, comprising administering to the patient an effective amount of an antibody or antigen-binding fragment of the claimed invention, wherein said antibody or antigen-binding fragment thereof blocks binding of OX40L to OX40 and/or inhibits one or more functions associated with binding of OX40L to OX40.

The present invention includes a method of treating a subject suffering from asthmatic symptoms comprising administering to a subject, e.g. a subject in need thereof, an amount of an antibody according to the claimed invention effective to reduce the asthmatic symptoms.

The antibody of the present invention may be administered by one or more of the routes including intravenous, intraperitoneal, inhalation, intramuscular, subcutaneous and oral routes. The present invention includes an inhalation device that delivers to a patient a therapeutically effective amount of an antibody according to the claimed invention.

The present invention includes a method for reducing the severity of asthma in a mammal comprising administering to the mammal a therapeutically effective amount of an anti-OX40 antibody having at least one of the following characteristics: the ability to bind human OX40 with a K_{D} between about 1×10¹⁰ to about 1×10¹²M; the ability to inhibit one or more functions associated with binding OX40 receptor and inhibiting binding of OX40L to said receptor.

In certain embodiments, the antibody binds human OX40 with a dissociation constant between about 1×10⁻¹² to about 1×10⁻¹¹M, 1×10⁻¹¹ to about 1×10⁻¹⁰M, 1×10⁻¹⁰ to about 1×10⁻⁹M, 1×10⁻⁹ to about 1×10⁻⁸M, 1×10⁻⁸ to about 1×10⁻⁷M.

The present invention includes a method for reducing the severity of asthma in a mammal, comprising administering to the mammal an effective amount of an antibody wherein the antibody comprises at least one of the following properties: (a) binds human OX40 with a K_{D} between about 1×10⁻¹² to about 1×10⁻⁸M; (b) inhibits one or more functions associated with binding OX40; and (c) inhibits binding of OX40L to OX40.

The antibodies of the present invention may also be useful for the treatment of, but are not limited to, allergy, asthma, atopic dermatitis multiple sclerosis, rheumatoid arthritis, psoriasis, inflammatory bowel disease, graft-versus-host disease, experimental autoimmune encephalomyelitis (EAE), autoimmune neuropathies (such as Guillain-Barré), autoimmune ureitis, autoimmune hemolytic anemia, autoimmune thrombocytopenia, myasthenia gravis, collagen-induced arthritis, colitis (such as ulcerative colitis), contact hypersensitivity reactions, diabetes, Crohn's Disease, and Grave's Disease.

The antibodies of the present invention may also be useful for the treatment of sarcoidosis, experimental leishmaniasis, pernicious anemia, temporal artertis, anti-phospholipid syndrome, vasculitides (such as Wegener's granulomatosis), Behcet's disease, dermatitis herpetiformis, pemphigus vulgaris, vitiligo, primary biliary cirrhosis, autoimmune hepatitis, Hashimoto's thyroiditis, autoimmune oophoritis and orchitis, autoimmune disease of the adrenal gland, systemic lupus erythematosis, scleroderma, dermatomyositis, polymysitis, dermatomyositis, spondyloarthropathies (such as ankylosing spondylitis), Reiter's Syndrome, Sjogren's syndrome, and others.

The present invention includes a use of the antibody of the present invention in the preparation of a medicament.

The present invention includes use of the antibody of the present invention for the treatment of a OX40 related disease or disorder.

The present invention includes use of the antibody of the present invention to detect OX40.

The present invention includes a kit comprising the antibody of the present invention in a predetermined amount in a container, and a buffer in a separate container.

The present invention includes a kit comprising the composition of the present invention in a predetermined amount in a container, and a buffer in a separate container.

The present invention includes an medical device comprising the antibody of the present invention.

The present invention includes a medical device comprising the composition of the present invention.

### 4. BRIEF DESCRIPTION OF THE FIGURES

FIG. 1A depicts the nucleic acid sequence for human OX40 cDNA (Accession No. NM_{—}004295.

FIG. 1B depicts the amino acid sequence for human OX40 protein (Accession No. NM_{—}003318.

FIG. 2 depicts the inhibitory effect of mouse anti-OX40 mAb B66 on the production of IL-2 (2A) and IL-13 (2B) by in vitro activated naïve human CD4+ T-cells.

FIG. 3 depicts the inhibitory effect of mouse anti-OX40 mAb B66 on the proliferation of in vitro activated naïve human CD4+ T-cells.

FIG. 4 depicts the apoptotic effect of chimeric anti-OX40 mAbs A10 and B66 on in vitro primed human Th2 cells.

FIG. 5 depicts the inhibitory effect of chimeric anti-OX40 mAbs A10 and B66 on the proliferation of in vitro primed human Th2 cells. Open Square represents naïve CD4; the Open Triangle represents naïve CD4+ with control L-cells; Solid Triangles represent B66 with naïve CD4+ and OX40L L-cells; Solid Squares represent 2C4 (IC) with naïve CD4+ and OX40L L-cells; and Solid Circles represent A10 with naïve CD4+ and OX40L-L-cells.

FIG. 6 depicts the inhibitory effect of chimeric anti-OX40 mAbs A10 and B66 on the production of IL2 (6A) and IL-13 (6B) by in vitro primed human Th2 cells. Open Square represents naïve CD4; the Open Triangle represents naïve CD4+ with control L-cells; Solid Triangles represent B66 with naïve CD4+ and OX40L L-cells; Solid Squares represent 2C4 (IC) with naïve CD4+ and OX40L L-cells; and Solid Circles represent A10 with naïve CD4+ and OX40L-L-cells.

FIG. 7: Table showing the comparison of the inhibitory effect of chimeric anti-OX40 mAb B66 and commercial anti-OX40 mAb L106 on the production of IL-2 and IL-13 by in vitro activated naïve human CD4+ T-cells.

FIG. 8: Table showing the inhibitory effect of humanized, chimeric and mouse anti-OX40 mAb A10 on the proliferation of in vitro activated naïve human CD4+ T-cells, as well as their production of IL-2, IL-5, and IL-13.

FIG. 9: Table showing the inhibitory effect of humanized, chimeric and mouse anti-OX40 mAb A10 on the proliferation of in vitro primed human Th1 cells, as well as their production of IL-2, IL-5, and IL-13.

FIG. 10: Table showing the inhibitory effect of humanized, chimeric and mouse anti-OX40 mAb A10 on the proliferation of in vitro primed human Th2 cells, as well as their production of IL-2. IL-5, and IL-13.

FIG. 11: Table showing the inhibitory effect of humanized, chimeric and mouse anti-OX40 mAb A10 on the proliferation of in vitro primed human Th2 cells, as well as their production of IL-2, IL-5, and IL-13 using allogenic TSLP activated myeloid dendritic cells.

FIG. 12A depicts the variable light chain amino acid sequence for murine antibodies A10 and B66 as compared with the human template IGKV4-1/IGKJ1 and the resulting humanized sequence of A10(TH)hu336F. Numbers in superscript indicate amino acid positions according to Kabat.

FIG. 12B depicts the variable heavy chain amino acid sequence for murine antibody A10 as compared with the human template IGHV1-46/IGHJ4 and the resulting humanized sequence of A10(TH)hu336F. Numbers in superscript indicate amino acid positions according to Kabat.

FIG. 13 depicts the higher binding activity of the humanized clone A10(TH)hu336F (▪) as compared to the parent murine antibody A10 () and it's chimeric form (▴).

FIG. 14 depicts nucleic acid sequences of the murine and humanized variable regions of anti-OX40: A) Light chain kappa variable 252-A10; B) Light chain kappa variable 252-B66; C) Humanized light chain variable A10 (SN), whole antibody B and E; D) Humanized light chain variable A10 (SH), whole antibody A and D; E) Humanized light chain variable A10(TH)hu336, whole antibody C and F; F) Heavy chain variable 252-A10; G) Heavy chain variable A10(TH)hu336, whole antibodies D-F; H) Heavy chain variable A10L70, whole antibodies A-C.

FIG. 15 shows that humanized clone A10(TH)hu336F (▪), murine A10 () and its chimeric form (▴) compete equally well for OX40.

FIGS. 16A,B & 17A,B depict exemplary acceptor human consensus framework sequences for use in practicing the instant invention with sequence identifiers as follows:

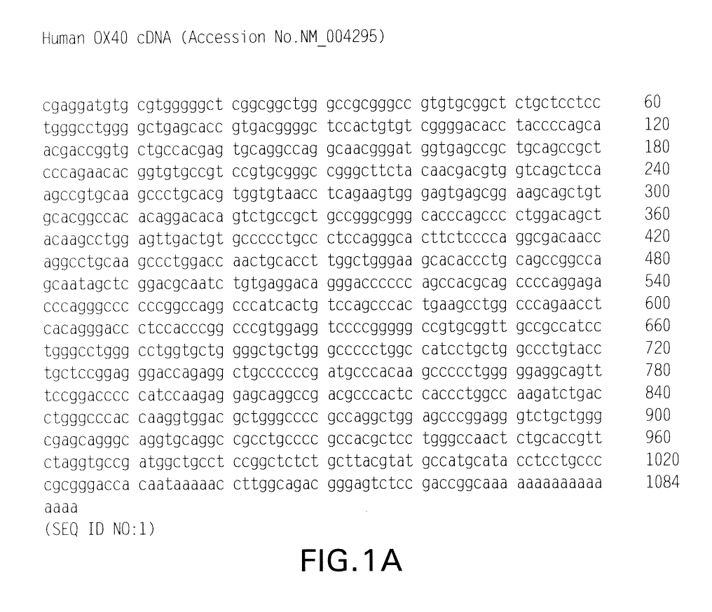

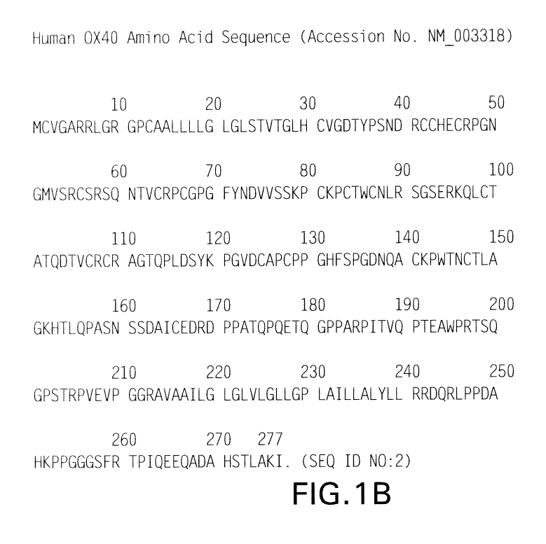

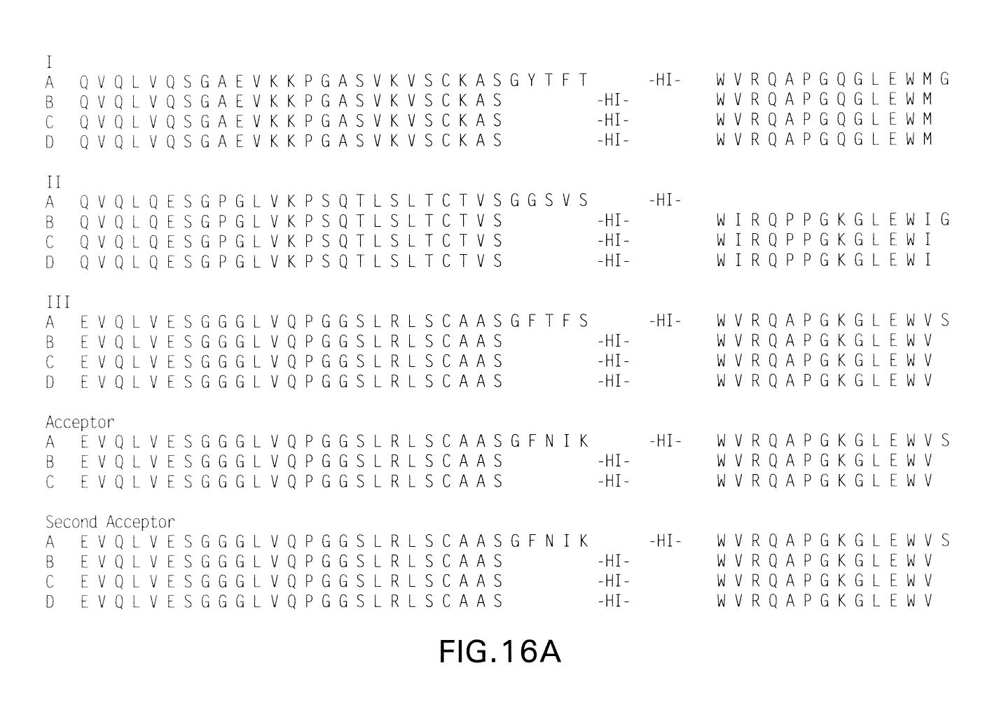

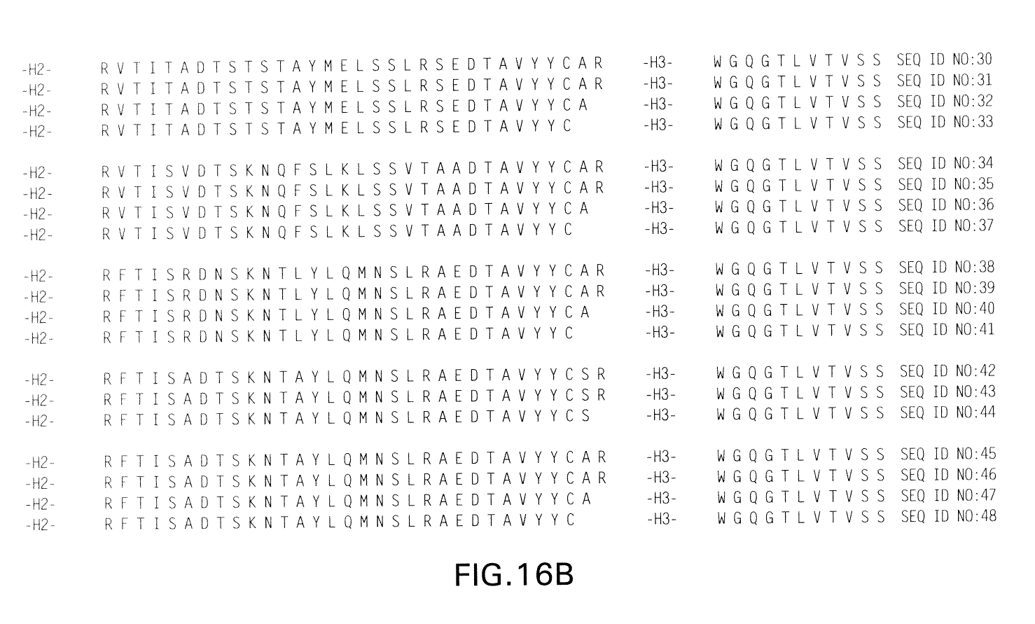

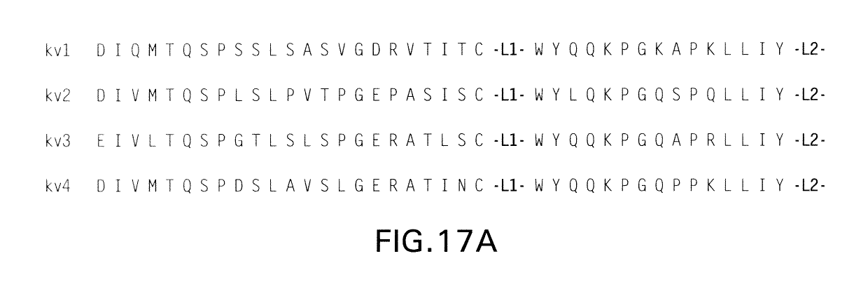

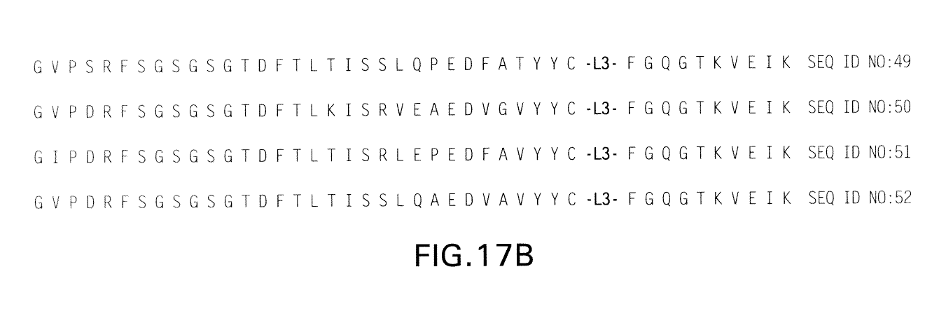

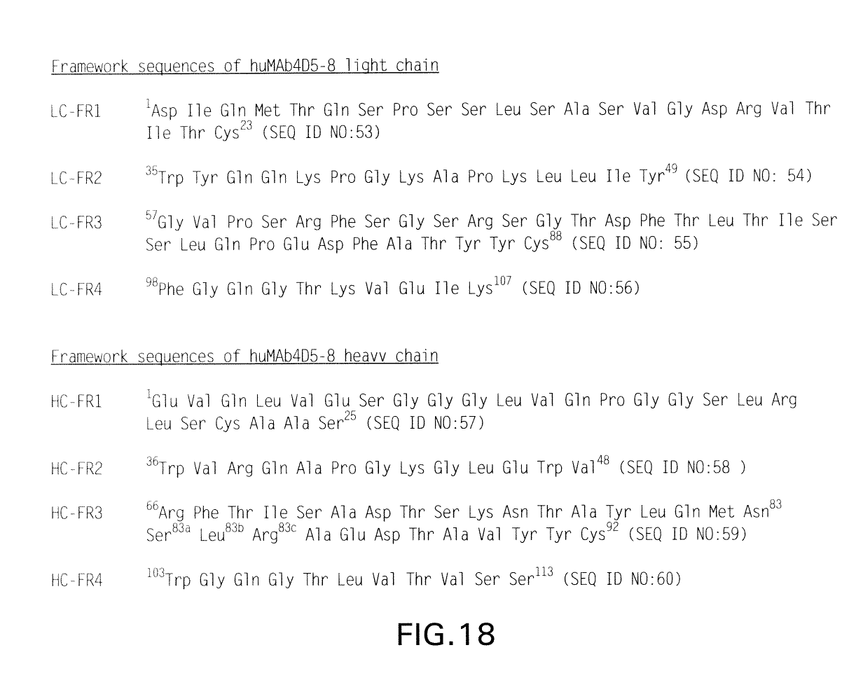

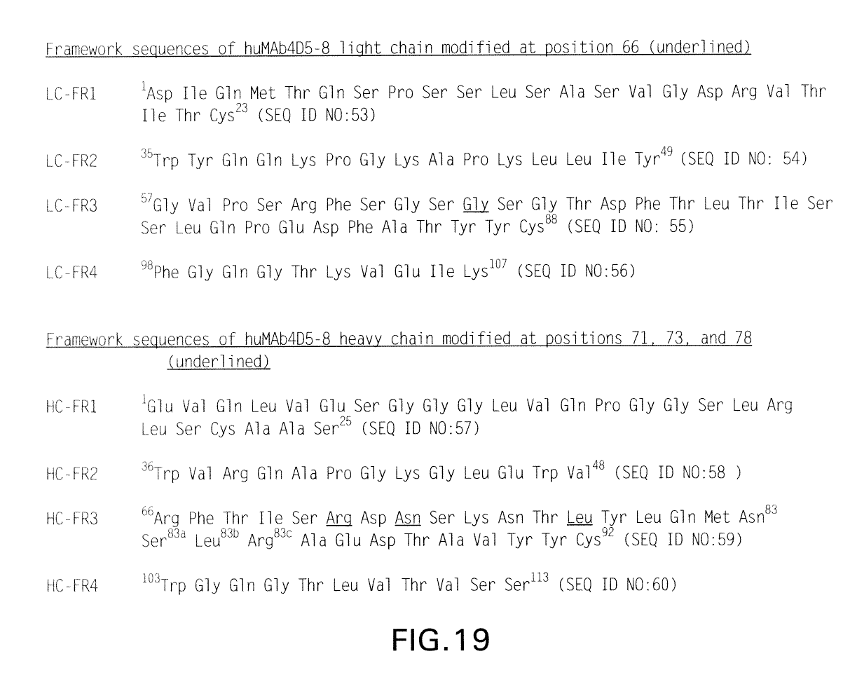

Variable heavy (VII) consensus frameworks (FIG. 16A, B) human VH subgroup I consensus framework minus Kabat CDRs (SEQ ID NO:30: human VH subgroup I consensus framework minus extended hypervariable regions (SEQ ID NOs:31-33); human VII subgroup II consensus framework minus Kabat CDRs (SEQ ID NO:34); human VII subgroup II consensus framework minus extended hypervariable regions (SEQ ID NOs:35-37); human VII subgroup II consensus framework minus extended human VII subgroup III consensus framework minus Kabat CDRs (SEQ ID NO:38); human VH subgroup III consensus framework minus extended hypervariable regions (SEQ ID NOs:39-41); human VH acceptor framework minus Kabat CDRs (SEQ ID NO:42); human VH acceptor framework minus extended hypervariable regions (SEQ ID NOs:43-44); human VH acceptor 2 framework minus Kabat CDRs (SEQ ID NO:45); human VH acceptor 2 framework minus extended hypervariable regions (SEQ ID NOs:46-48); variable light (VL) consensus frameworks (FIG. 17A,B); human VL kappa subgroup I consensus framework (SEQ ID NO:49); human VL kappa subgroup II consensus framework (SEQ ID NO:50); human VL kappa subgroup III consensus framework (SEQ ID NO:51); human VL kappa subgroup IV consensus framework (SEQ ID NO:52).

FIG. 18 depicts framework region sequences of huMAb4D5-8 light and heavy chains. Numbers in superscript/bold indicate amino acid positions according to Kabat.

FIG. 19 depicts modified/variant framework region sequences of huMAb4D5-8 light and heavy chains. Numbers in superscript/bold indicate amino acid positions according to Kabat.

### 5. DETAILED DESCRIPTION

This invention is not limited to the particular methodology, protocols, cell lines, vectors, or reagents described herein because they may vary. Further, the terminology used herein is for the purpose of describing particular embodiments only and is not intended to limit the scope of the present invention. As used herein and in the appended claims, the singular forms “a”, “an”, and “the” include plural reference unless the context clearly dictates otherwise, e.g., reference to “a host cell” includes a plurality of such host cells. Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

All patents and publications mentioned herein are incorporated herein by reference to the extent allowed by law for the purpose of describing and disclosing the proteins, enzymes, vectors, host cells, and methodologies reported therein that might be used with the present invention. However, nothing herein is to be construed as an admission that the invention is not entitled to antedate such disclosure by virtue of prior inventions.

### 5.1 DEFINITIONS

Terms used throughout this application are to be construed with ordinary and typical meaning to those of ordinary skill in the art. However. Applicants desire that the following terms be given the particular definition as defined below.

The phrase “substantially identical” with respect to an antibody chain polypeptide sequence may be construed as an antibody chain exhibiting at least 70%, or 80%, or 90%, or 95% sequence identity to the reference polypeptide sequence. The term with respect to a nucleic acid sequence may be construed as a sequence of nucleotides exhibiting at least about 85%, or 90%, or 95%, or 97% sequence identity to the reference nucleic acid sequence.

The term “identity” or “homology” shall be construed to mean the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions shall be construed as reducing identity or homology. Methods and computer programs for the alignment are well known in the art. Sequence identity may be measured using sequence analysis software.

The term “antibody” is used in the broadest sense, including immunoglobulin molecules and immunologically active portions of immunoglobulin molecules, i.e., molecules that contain an antigen binding site that immunospecifically binds an antigen, and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end. Moreover, the term “antibody” (Ab) or “monoclonal antibody” (mAb) is meant to include both intact molecules, as well as, antibody fragments (such as, for example, Fab and F(ab′)₂ fragments) which are capable of specifically binding to a protein. Fab and F(ab′)₂ fragments lack the Fc fragment of intact antibody, clear more rapidly from the circulation of the animal or plant, and may have less non-specific tissue binding than an intact antibody (Wahl, et al., J Nucl Med 24:316 (1983)).

As used herein, “anti-OX40 antibody” means an antibody which binds to human OX40 in such a manner so as to inhibit or substantially reduce the binding of such OX40 to its ligand, OX40 ligand.

As used herein, the term “OX40-mediated disorder” include conditions associated with allergy, asthma, and diseases associated with autoimmunity and inflammation, which include multiple sclerosis, rheumatoid arthritis, inflammatory bowel disease, graft-versus-host disease, experimental autoimmune encephalomyelitis (EAE), experimental leishmaniasis, collagen-induced arthritis, colitis (such as ulcerative colitis), contact hypersensitivity reactions, diabetes, Crohn's Disease, and Grave's Disease. Other conditions such as sarcoidosis, autoimmune ocular diseases, autoimmune uveitis, atopic dermatitis, myasthenia gravis, autoimmune neuropathies (such as Guillain-Barré), autoimmune ureitis, autoimmune hemolytic anemia, pernicious anemia, autoimmune thrombocytopenia, temporal artertis, anti-phospholipid syndrome, vasculitides (such as Wegener's granulomatosis), Behcet's disease, psoriasis, dermatitis herpetiformis, pemphigus vulgaris, vitiligo, primary biliary cirrhosis, autoimmune hepatitis, Hashimoto's thyroiditis, autoimmune oophoritis and orchitis, autoimmune disease of the adrenal gland, systemic lupus erythematosis, scleroderma, dermatomyositis, polymysitis, dermatomyositis, spondyloarthropathies (such as ankylosing spondylitis), Reiter's Syndrome, and Sjogren's syndrome are also encompassed under the scope of this term.

The term “variable” in the context of variable domain of antibodies, refers to the fact that certain portions of the variable domains differ extensively in sequence among antibodies and are used in the binding and specificity of each particular antibody for its particular target. However, the variability is not evenly distributed through the variable domains of antibodies. It is concentrated in three segments called complementarity determining regions (CDRs) also known as hypervariable regions both in the light chain and the heavy chain variable domains. The more highly conserved portions of variable domains are called the framework (FR). As is known in the art, the amino acid position/boundary delineating a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The invention provides antibodies comprising modifications in these hybrid hypervariable positions. The variable domains of native heavy and light chains each comprise four FR regions, largely a adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the target binding site of antibodies (see Kabat, et al. Sequences of Proteins of Immunological Interest, National Institute of Health, Bethesda, Md. (1987)). As used herein, numbering of immunoglobulin amino acid residues is done according to the immunoglobulin amino acid residue numbering system of Kabat, et al., unless otherwise indicated.

The term “antibody fragment” refers to a portion of a full-length antibody, generally the target binding or variable region. Examples of antibody fragments include Fab, Fab′, F(ab′)₂ and Fv fragments. The phrase “antigen binding fragment” of an antibody is a compound having qualitative biological activity in common with a full-length antibody. For example, an antigen binding fragment of an anti-OX40 antibody is one which can bind to an OX40 receptor in such a manner so as to prevent or substantially reduce the ability of such molecule from having the ability to bind to the OX40 ligand. As used herein, “functional fragment” with respect to antibodies, refers to Fv, F(ab) and F(ab′)₂ fragments. An “Fv” fragment is the minimum antibody fragment which contains a complete target recognition and binding site. This region consists of a dimer of one heavy and one light chain variable domain in a tight, non-covalent association (V_{H}-V_{L} dimer). It is in this configuration that the three CDRs of each variable domain interact to define a target binding site on the surface of the V_{H}-V_{L} dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for target binding.

The Fab fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single target site. Furthermore, in contrast to conventional (polyclonal) antibody preparations which typically include different antibodies directed against different determinants (epitopes), each monoclonal antibody is directed against a single determinant on the target. In addition to their specificity, monoclonal antibodies are advantageous in that they may be synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies for use with the present invention may be isolated from phage antibody libraries using the well known techniques. The parent monoclonal antibodies to be used in accordance with the present invention may be made by the hybridoma method first described by Kohler, et al., Nature 256:495 (1975), or may be made by recombinant methods.

The term “chimeric” antibody as used herein refers to an antibody having variable sequences derived from a non-human immunoglobulins, such as rat or mouse antibody, and human immunoglobulins constant regions, typically chosen from a human immunoglobulin template.

“Humanized” forms of non-human (e.g. murine) antibodies are chimeric immunoglobulins, immunoglobulin chains or fragments thereof (such as Fv, Fab, Fab′, F(ab′)₂ or other target-binding subsequences of antibodies) which contain minimal sequence derived from non-human immunoglobulin. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fe), typically that of a human immunoglobulin template chosen.

As used herein, “human antibodies” include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati, et al.

The terms “cell,” “cell line,” and “cell culture” include progeny. It is also understood that all progeny may not be precisely identical in DNA content, due to deliberate or inadvertent mutations. Variant progeny that have the same function or biological property, as screened for in the originally transformed cell, are included. The “host cells” used in the present invention generally are prokaryotic or eukaryotic hosts.

“Transformation” of a cellular organism with DNA means introducing DNA into an organism so that the DNA is replicable, either as an extrachromosomal element or by chromosomal integration. “Transfection” of a cellular organism with DNA refers to the taking up of DNA, e.g., an expression vector, by the cell or organism whether or not any coding sequences are in fact expressed. The terms “transfected host cell” and “transformed” refer to a cell in which DNA was introduced. The cell is termed “host cell” and it may be either prokaryotic or eukaryotic. Typical prokaryotic host cells include various strains of E. coli. Typical eukaryotic host cells are mammalian, such as Chinese hamster ovary or cells of human origin. The introduced DNA sequence may be from the same species as the host cell of a different species from the host cell, or it may be a hybrid DNA sequence, containing some foreign and some homologous DNA.

The term “vector” means a DNA construct containing a DNA sequence which is operably linked to a suitable control sequence capable of effecting the expression of the DNA in a suitable host. Such control sequences include a promoter to effect transcription, an optional operator sequence to control such transcription, a sequence encoding suitable mRNA ribosome binding sites, and sequences which control the termination of transcription and translation. The vector may be a plasmid, a phage particle, or simply a potential genomic insert. Once transformed into a suitable host, the vector may replicate and function independently of the host genome, or may in some instances, integrate into the genome itself. In the present specification. “plasmid” and “vector” are sometimes used interchangeably, as the plasmid is the most commonly used form of vector. However, the invention is intended to include such other forms of vectors which serve equivalent function as and which are, or become, known in the art.

“Mammal” for purposes of treatment refers to any animal classified as a mammal, including human, domestic and farm animals, nonhuman primates, and zoo, sports, or pet animals, such as dogs, horses, cats, cows, etc.

The word “label” when used herein refers to a detectable compound or composition which can be conjugated directly or indirectly to a molecule or protein, e.g., an antibody. The label may itself be detectable (e.g., radioisotope labels or fluorescent labels) or, in the case of an enzymatic label, may catalyze chemical alteration of a substrate compound or composition which is detectable.

As used herein, “solid phase” means a non-aqueous matrix to which the antibody of the present invention can adhere. Example of solid phases encompassed herein include those formed partially or entirely of glass (e.g. controlled pore glass), polysaccharides (e.g., agarose), polyacrylamides, polystyrene, polyvinyl alcohol, and silicones. In certain embodiments, depending on the context, the solid phase can comprise the well of an assay plate; in others it is a purification column (e.g. an affinity chromatography column).

### 5.2 IMMUNOGEN

Two forms of the OX40 receptor were used as immunogen to generate anti-OX40 antibodies of the present invention: (1) one was the soluble form of the human OX40 receptor expressed as fusion protein comprising a human Fcγ1 and the extracellular domain encoded by nucleotide residues 105 to 627; and (2) the second was a cell-based immunogen comprising the full length OX40 expressed on the surface of stably transfected mouse fibroblast L-cells. Soluble OX40 receptor or fragments thereof may be used as immunogens for generating the antibodies of the present invention. Resulting antagonist antibodies are directed against OX40 and capable of inhibiting OX40L from interacting with OX40 thereby neutralizing receptor activity. Preferably, the immunogen is a polypeptide, and may be a transmembrane molecule. The immunogen may be produced recombinantly or made using synthetic methods. The immunogen may also be isolated from a natural source.

The immunogen may comprise the extracellular domain of OX40. Alternatively, cells expressing a transmembrane protein comprising the extracellular domain may be used as the immunogen. Such cells can be derived from a natural source (e.g., T-cell lines) or may be cells which have been transformed by recombinant techniques to express the receptor on their surface. Other immunogens and forms thereof useful for generating antibodies will be apparent to those in the art. Immunizing a host animal, such as a rodent, with a whole cell immunogen is well known in the art.

Alternatively, a gene or a cDNA encoding OX40 may be cloned into a plasmid or other expression vector and expressed in any of a number of expression systems according to methods well known to those of skill in the art. Methods of cloning and expressing OX40 and the nucleic acid sequence OX40 are well known (see, for example, U.S. Pat. Nos. 5,821,332 and 5,759.546). Because of the degeneracy of the genetic code, a multitude of nucleotide sequences encoding OX40 polypeptides may be produced. One may vary the nucleotide sequence by selecting combinations based on possible codon choices. These combinations are made in accordance with the standard triplet genetic code as applied to the nucleotide sequence that codes for naturally occurring OX40 polypeptide and all such variations are to be considered. Any one of these polypeptides may be used in the immunization of an animal to generate antibodies that bind to OX40.

The immunogen OX40 polypeptide may, when beneficial, be expressed as a fusion protein that has the OX40 polypeptide fused to another polypeptide, such as an immunoglobulins Fc region. The fused polypeptide often aids in protein purification, e.g., by permitting the fusion protein to be isolated and purified by affinity chromatography. Fusion proteins can be produced by culturing a recombinant cell transformed with a fusion nucleic acid sequence that encodes a protein including the fusion segment attached to either the carboxyl and/or amino terminal end of the protein. Fusion segments may include, but are not limited to, immunoglobulin Fc regions, glutathione-S-transferase, β-galactosidase, a poly-histidine segment capable of binding to a divalent metal ion, and maltose binding protein.

In the present invention, recombinant OX40 was used to immunize mice to generate the antagonist antibodies. Recombinant OX40 is commercially available from a number of sources (see, e.g, R & D Systems, Minneapolis, Minn., PeproTech, Inc., NJ, and Sanofi Bio-Industries, Inc., Tervose, Pa.) Exemplary polypeptides comprise all or a portion of SEQ ID NO.1 and variants thereof.

### 5.3 ANTIBODY GENERATION

The antibodies of the present invention may be generated by any suitable method known in the art. The antibodies of the present invention may comprise polyclonal antibodies. Methods of preparing polyclonal antibodies are known to the skilled artisan (Harlow, et al., Antibodies: a Laboratory Manual, Cold spring Harbor Laboratory Press, 2nd ed. (1988)), which is hereby incorporated herein by reference in its entirety).

For example, an immunogen as described above may be administered to various host animals including, but not limited to, rabbits, mice, rats, etc., to induce the production of sera containing polyclonal antibodies specific for the antigen. The administration of the immunogen may entail one or more injections of an immunizing agent and, if desired, an adjuvant. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Additional examples of adjuvants which may be employed include the MPL-TDM adjuvant (monophosphoryl lipid A, synthetic trehalose dicorynomycolate). Immunization protocols are well known in the art in the art and may be performed by any method that elicits an immune response in the animal host chosen. Adjuvants are also well known in the art.

Typically, the immunogen (with or without adjuvant) is injected into the mammal by multiple subcutaneous or intraperitoneal injections, or intramuscularly or through IV. The immunogen may include an OX40 polypeptide, a fusion protein, or variants thereof. Depending upon the nature of the polypeptides (i.e., percent hydrophobicity, percent hydrophilicity, stability, net charge, isoelectric point etc.), it may be useful to conjugate the immunogen to a protein known to be immunogenic in the mammal being immunized. Such conjugation includes either chemical conjugation by active derivation of chemical functional groups to both the immunogen and the immunogenic protein to be conjugated such that a covalent bond is formed, or through fusion-protein based methodology, or other methods known to the skilled artisan. Examples of such immunogenic proteins include, but are not limited to, keyhole limpet hemocyanin, ovalbumin, serum albumin, bovine thyroglobulin, soybean trypsin inhibitor, and promiscuous T helper peptides. Various adjuvants may be used to increase the immunological response as described above.

The antibodies of the present invention may comprise monoclonal antibodies. Monoclonal antibodies are antibodies which recognize a single antigenic site. Their uniform specificity makes monoclonal antibodies much more useful than polyclonal antibodies, which usually contain antibodies that recognize a variety of different antigenic sites. Monoclonal antibodies may be prepared using hybridoma technology, such as those described by Kohler, et al., Nature 256:495 (1975); U.S. Pat. No. 4,376,110; Harlow, et al. Antibodies: A Laboratory Manual, Cold Spring Harbor Laboratory Press, 2nd ed. (1988) and Hammerling, et al., Monoclonal Antibodies and T-Cell Hybridomas, Elsevier (1981), recombinant DNA methods, or other methods known to the artisan. Other examples of methods which may be employed for producing monoclonal antibodies include, but are not limited to, the human B-cell hybridoma technique (Kosbor, et al., Immunology Today 4:72 (1983); Cole, et al., Proc Natl Sci USA 80:2026 (1983)), and the EBV-hybridoma technique (Cole, et al., Monoclonal Antibodies and Cancer Therapy, pp. 77-96. Alan R. Liss (1985)). Such antibodies may be of any immunoglobulin class including IgG, IgM, IgE, IgA. IgD and any subclass thereof. The hybridoma producing the mAb of this invention may be cultivated in vitro or in vivo.

In the hybridoma model, a host such as a mouse, a humanized mouse, a mouse with a human immune system, hamster, rabbit, camel, or any other appropriate host animal, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, Academic Press, pp. 59-103 (1986)).

Generally, in making antibody-producing hybridomas, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, pp. 59-103 (1986)). Immortalized cell lines are usually transformed mammalian cells, particularly myeloma cells of rodent, bovine or human origin. Typically, a rat or mouse myeloma cell line is employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), substances that prevent the growth of HGPRT-deficient cells.

Preferred immortalized cell lines are those that fuse efficiently, support stable high-level production of antibody by the selected antibody-producing cells, and are sensitive to a medium such as FIAT medium. Among these myeloma cell lines are murine myeloma lines, such as those derived from the MOPC-21 and MPC-11 mouse tumors available from the Salk Institute Cell Distribution Center. San Diego. Calif. U.S. Application No., and SP2/0 or X63-Ag8-653 cells available from the American Type Culture Collection. Rockville, Md. USA. Human myeloma and mouse-human heteromyeloma cell lines also have been described for the production of human monoclonal antibodies (Kozbor, J Immunol 133:3001 (1984); Brodeur, et al., Monoclonal Antibody Production Techniques and Applications, Marcel Dekker, Inc. pp. 51-63 (1987)). The mouse myeloma cell line NSO may also be used (European Collection of Cell Cultures, Salisbury, Wilshire, UK).

The culture medium in which hybridoma cells are grown is assayed for production of monoclonal antibodies directed against OX40. The binding specificity of monoclonal antibodies produced by hybridoma cells may be determined by immunoprecipitation or by an in vitro binding assay, such as radioimmunoassay (RIA) or enzyme-linked immunoabsorbent assay (ELISA). Such techniques are known in the art and within the skill of the artisan. The binding affinity of the monoclonal antibody to OX40 can, for example, be determined by a Scatchard analysis (Munson, et al., Anal Biochem 107:220 (1980)).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, Academic Press, pp. 59-103 (1986)). Suitable culture media for this purpose include, for example, Dulbecco's Modified Eagle's Medium (D-MEM) or RPMI-1640 medium. In addition, the hybridoma cells may be grown in vivo as ascites tumors in an animal.

The monoclonal antibodies secreted by the subclones are suitably separated or isolated from the culture medium, ascites fluid, or serum by conventional immunoglobulin purification procedures such as, for example, protein A-Sepharose, hydroxylapatite chromatography, gel exclusion chromatography, gel electrophoresis, dialysis, or affinity chromatography.

A variety of methods exist in the art for the production of monoclonal antibodies and thus, the invention is not limited to their sole production in hybridomas. For example, the monoclonal antibodies may be made by recombinant DNA methods, such as those described in U.S. Pat. No. 4,816,567. The hybridoma cells serve as a source of such DNA. Once isolated, the DNA may be placed into expression vectors, which are then transfected into host cells such as E. coli cells, NS0 cells, Simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison, et al., Proc Natl Acad Sci USA 81:6851 (1984)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain cross-linking. Alternatively, the relevant cysteine residues are substituted with another amino acid residue or are deleted so as to prevent cross-linking.

Antibody fragments which recognize specific epitopes may be generated by known techniques. Traditionally, these fragments were derived via proteolytic digestion of intact antibodies (see, e.g., Morimoto, et al., J Biochem Biophys Methods 24:107 (1992); Brennan, et al., Science 229:81 (1985)). For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain. However, these fragments can now be produced directly by recombinant host ells. For example, the antibody fragments can be isolated from an antibody phage library. Alternatively, F(ab′)₂-SH fragments can be directly recovered from E. coli and chemically coupled to form F(ab′), fragments (Carter, et al., Bio/Technology 10:163 (1992). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (Fv) (PCT patent application WO 93/16185).

For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi, et al., BioTechniques 4:214 (1986); Gillies, et al., J Immunol Methods 125:191 (1989); U.S. Pat. Nos. 5,807,715; 4,816,567; and 4,816,397, which are incorporated herein by reference in their entirety.

A humanized antibody is designed to have greater homology to a human immunoglobulin than animal-derived monoclonal antibodies. Humanization is a technique for making a chimeric antibody wherein substantially less than an intact human variable domain has been substituted by the corresponding sequence from a non-human species. Humanized antibodies are antibody molecules generated in a non-human species that bind the desired antigen having one or more complementarity determining regions (CDRs) from the non-human species and framework (FR) regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., U.S. Pat. No. 5,585,089; Riechmann, et al., Nature 332:323 (1988), which are incorporated herein by reference in their entireties. Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28:489 (1991); Studnicka, et al., Protein Engineering 7:805 (1994); Roguska, et al., Proc Natl Acad Sci USA 91:969 (1994)), and chain shuffling (U.S. Pat. No. 5,565,332).

Generally, a humanized antibody has one or more amino acid residues introduced into it from a source that is non-human. These non-human amino acid residues are often referred to as “import” residues, which are typically taken from an “import” variable domain. Humanization can be essentially performed following the methods of Winter and co-workers (Jones, et al., Nature 321:522 (1986); Riechmann, et al., Nature 332:323 (1988); Verhoeyen, et al., Science 239:1534 (1988)), by substituting non-human CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possible some FR residues are substituted from analogous sites in rodent antibodies.

It is further important that humanized antibodies retain higher affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of certain residues in the functioning of the candidate immunoglobulin sequence, i.e., the analysis of residues that influence the ability of the candidate immunoglobulin sequences, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is maximized, although it is the CDR residues that directly and most substantially influence antigen binding.

The choice of human variable domains, both light and heavy, to be used in making the humanized antibodies is important to reduce antigenicity. According to the so-called “best-fit” method, the sequence of the variable domain of a non-human antibody is screened against the entire library of known human variable-domain sequences. The human sequence which is closest to that of that of the non-human parent antibody is then accepted as the human FR for the humanized antibody (Sims, et al., J Immunol 151:2296 (1993); Chothia, et al., J Mol Biol 196:901 (1987)).

Another method uses a particular framework derived from the consensus sequence of all human antibodies of a particular subgroup of light or heavy chains. The same framework may be used for several different humanized antibodies (Carter, et al., Proc Natl Acad Sci USA 89:4285 (1992); Presta, et al., J Immunol 151:2623 (1993)). An antibody of the invention can comprise any suitable human or human consensus light or heavy chain framework sequences, provided that the antibody exhibits the desired biological characteristics (e.g., a desired binding affinity). In some embodiments, one or more (such as 2, 3, 4, 5, 6, 7, 8, 9, or more) additional modifications are present within the human and/or human consensus non-hypervariable region sequences. In one embodiment, an antibody of the invention comprises at least a portion (or all) of the framework sequence of human light chain. In one embodiment, an antibody of the invention comprises at least a portion (or all) of the framework sequence of human heavy chain. In one embodiment, an antibody of the invention comprises at least a portion (or all) of human subgroup I framework consensus sequence. In some embodiments, antibodies of the invention comprise a human subgroup III heavy chain framework consensus sequence. In one embodiment, the framework consensus sequence of the antibody of the invention comprises substitution at position 71, 73 and/or 78. In some embodiments of these antibodies, position 71 is A, 73 is T and/or 78 is A.

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety. The techniques of Cole, et al. and Boerner, et al. are also available for the preparation of human monoclonal antibodies (Cole, et al., Monoclonal Antibodies and Cancer Therapy, Alan R. Riss (1985); and Boerner, et al., J Immunol 147:86 (1991)).

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. See, e.g., Jakobovitis, et al., Proc Acad Sci USA 90:2551 (1993); Jakobovitis, et al., Nature 362:255 (1993); Bruggermann, et al., Year in Immunol 7:33 (1993); Duchosal, et al., Nature 355:258 (1992)).

The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA. IgM and IgE antibodies. For an overview of this technology for producing human antibodies, see Lonberg, et al., Int Rev Immunol 13:65-93 (1995). For a detailed discussion of this technology for producing human antibodies and human monoclonal antibodies and protocols for producing such antibodies, see, e.g. PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.), Genpharm (San Jose, Calif.), and Medarex, Inc. (Princeton, N.J.) can be engaged to provide human antibodies directed against a selected antigen using technology similar to that described above.

Also human mAbs could be made by immunizing mice transplanted with human peripheral blood leukocytes, splenocytes or bone marrows (e.g., Trioma techniques of XTL). Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers, et al., Bio/technology 12:899 (1988)).

Further, antibodies to the polypeptides of the invention can, in turn, be utilized to generate anti-idiotype antibodies that “mimic” polypeptides of the invention using techniques well known to those skilled in the art (See, e.g., Greenspan, et al., FASEB J 7:437 (1989); Nissinoff, J Immunol 147:2429 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide of the invention and/or to bind its ligands/receptors, and thereby block its biological activity.

The antibodies of the present invention may be bispecific antibodies. Bispecific antibodies are monoclonal, preferably human or humanized, antibodies that have binding specificities for at least two different antigens. In the present invention, one of the binding specificities may be directed towards OX40, the other may be for any other antigen, and preferably for a cell-surface protein, receptor, receptor subunit, tissue-specific antigen, virally derived protein, virally encoded envelope protein, bacterially derived protein, or bacterial surface protein, etc.

Methods for making bispecific antibodies are well known. Traditionally, the recombinant production of bispecific antibodies is based on the co-expression of two immunoglobulin heavy-chain/light-chain pairs, where the two heavy chains have different specificities (Milstein, et al., Nature 305:537 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829 and in Traunecker, et al., EMBO J 10:3655 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It may have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transformed into a suitable host organism. For further details of generating bispecific antibodies see, for example Suresh, et al., Meth In Enzym 121:210 (1986).

Heteroconjugate antibodies are also contemplated by the present invention. Heteroconjugate antibodies are composed of two covalently joined antibodies. Such antibodies have, for example, been proposed to target immune system cells to unwanted cells (U.S. Pat. No. 4,676,980). It is contemplated that the antibodies may be prepared in vitro using known methods in synthetic protein chemistry, including those involving cross-linking agents. For example, immunotoxins may be constructed using a disulfide exchange reaction or by forming a thioester bond. Examples of suitable reagents for this purpose include iminothiolate and methyl-4-mercaptobutyrimidate and those disclosed, for example, in U.S. Pat. No. 4,676,980.

In addition, one can generate single-domain antibodies to OX40. Examples of this technology have been described in WO9425591 for antibodies derived from Camelidae heavy chain Ig, as well in US20030130496 describing the isolation of single domain fully human antibodies from phage libraries.

One can also create a single peptide chain binding molecules in which the heavy and light chain Fv regions are connected. Single chain antibodies (“scFv”) and the method of their construction are described in U.S. Pat. No. 4,946,778. Alternatively, Fab can be constructed and expressed by similar means. All of the wholly and partially human antibodies are less immunogenic than wholly murine mAbs, and the fragments and single chain antibodies are also less immunogenic.

Antibodies or antibody fragments can be isolated from antibody phage libraries generated using the techniques described in McCafferty, et al., Nature 348:552 (1990). Clarkson, et al., Nature 352:624 (1991) and Marks, et al., J Biol 222:581 (1991) describe the isolation of murine and human antibodies, respectively, using phage libraries. Subsequent publications describe the production of high affinity (nM range) human antibodies by chain shuffling (Marks, et al., Bio/Technology 10:779 (1992)), as well as combinatorial infection and in vivo recombination as a strategy for constructing very large phage libraries (Waterhouse, et al., Nuc Acids Res 21:2265 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc Natl Acad Sci USA 81:6851 (1984)).

Another alternative is to use electrical fusion rather than chemical fusion to form hybridomas. This technique is well established. Instead of fusion, one can also transform a B cell to make it immortal using, for example, an Epstein Barr Virus, or a transforming gene. See, e.g., “Continuously Proliferating Human Cell Lines Synthesizing Antibody of Predetermined Specificity,” Zurawaki, et al., in Monoclonal Antibodies, ed. by Kennett, et al., Plenum Press, pp. 19-33. (1980)). Anti-OX40 mAbs can be raised by immunizing rodents (e.g., mice, rats, hamsters, and guinea pigs) with OX40 protein, fusion protein, or its fragments expressed by either eukaryotic or prokaryotic systems. Other animals can be used for immunization, e.g., non-human primates, transgenic mice expression immunoglobulins, and severe combined immunodeficient (SCID) mice transplanted with human B lymphocytes. Hybridomas can be generated by conventional procedures by fusing B lymphocytes from the immunized animals with myeloma cells (e.g., Sp2/0 and NSO), as described earlier (Köhler, et al., Nature 256:495 (1975)). In addition, anti-OX40 antibodies can be generated by screening of recombinant single-chain Fv or Fab libraries from human B lymphocytes in phage-display systems. The specificity of the mAbs to OX40 can be tested by ELISA, Western immunoblotting, or other immunochemical techniques. The inhibitory activity of the antibodies on CD4+ T cell activation can be assessed by proliferation, cytokine release, and apoptosis assays. The hybridomas in the positive wells are cloned by limiting dilution. The antibodies are purified for characterization for specificity to human OX40 by the assays described above.

### 5.4 IDENTIFICATION OF ANTAGONIST ANTI-OX40 ANTIBODIES

The present invention provides antagonist monoclonal antibodies that inhibit and neutralize the action of OX40. In particular, the antibodies of the present invention bind to and inhibit the activation of OX40. The antibodies of the present invention include the antibodies designated A10 and B66 and humanized antibodies of these murine antibodies are disclosed. The present invention also includes antibodies that bind to the same epitope as one of these antibodies, e.g., that of monoclonal antibody A10(TH)hu336F.

Candidate anti-OX40 antibodies were screened using: (1) fluorometric micro-volume assay technology (FMAT) (Applied Biosystem, CA), (2) an enzyme-linked immuno-absorbent assay (ELISA), and (3) a flow cytometry immunoassay. Assays performed to characterize the chosen antibodies included: (1) Hut-78 Assay; (2) Naïve CD4+ T-cell Assay; (3) TH1, TH2 and TH17 conditions; (4) TCR-activated naïve CD4+ T-cell protocol; (5) Apoptosis assay, and (6) cross-reactivity testing. Experimental details are described in the Examples.

Antibodies of the invention include, but are not limited to, polyclonal, monoclonal, monovalent, bispecific, heteroconjugate, multispecific, human, humanized or chimeric antibodies, single chain antibodies, single-domain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above.

The immunoglobulin molecules of the invention can be of any type (e.g., IgG, IgE, IgM, IgD, NA and IgY), class (e.g., IgG1, IgG2, IgG3, IgG4, IgA1 and IgA2) or subclass of immunoglobulin molecule. The antibodies may be antigen-binding antibody fragments of the present invention and include, but are not limited to, Fab, Fab′ and F(ab′)₂, Fd, single-chain Fvs (scFv), single-chain antibodies, disulfide-linked Fvs (sdFv) and single-domain antibodies comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments comprising any combination of variable region(s) with a hinge region, CH1, CH2, and CH3 domains. The antibodies or the invention may be from any animal origin including birds and mammals. Preferably, the antibodies are from human, non-human primates, rodents (e.g., mouse and rat), donkey, sheep, rabbit, goat, guinea pig, camel, horse, or chicken.

The antibodies of the present invention may be monospecific, bispecific, trispecific or of greater multispecificity. Multispecific antibodies may be specific for different epitopes of OX40 or may be specific for both OX40 as well as for a heterologous epitope, such as a heterologous polypeptide or solid support material. See, e.g., PCT publications WO 93/17715; WO 92/08802; WO 91/00360; WO 92/05793; Tutt, et al., J Immunol 147:60 (1991); U.S. Pat. Nos. 4,474,893; 4,714,681; 4,925,648; 5,573,920; 5,601,819; Kostelny, et al., J Immunol 148:1547 (1992).

Antibodies of the present invention may be described or specified in terms of the epitope(s) or portion(s) of OX40 which they recognize or specifically bind. The epitope(s) or polypeptide portion(s) may be specified as described herein, e.g., by N-terminal and C-terminal positions, by size in contiguous amino acid residues, or listed in the Tables and Figures.

Antibodies of the present invention may also be described or specified in terms of their cross-reactivity. Antibodies that bind OX40 polypeptides, which have at least 95%, at least 90%, at least 85%, at least 80%, at least 75%, at least 70%, at least 65%, at least 60%, at least 55%, and at least 50% identity (as calculated using methods known in the art and described herein) to OX40 are also included in the present invention. Anti-OX40 antibodies may also bind with a K_{D} of less than about 10⁻⁷ M, less than about 10⁻⁶ M, or less than about 10⁻⁵ M to other proteins, such as anti-OX40 antibodies from species other than that against which the anti-OX40 antibody is directed.

In specific embodiments, antibodies of the present invention may cross-react with other mammalian homologues of human OX40 and the corresponding epitopes thereof. In a specific embodiment, the above-described cross-reactivity is with respect to any single specific antigenic or immunogenic polypeptide or combination(s) of the specific antigenic and/or immunogenic polypeptides disclosed herein.

Further included in the present invention are antibodies which bind polypeptides encoded by polynucleotides which hybridize to a polynucleotide encoding OX40 under stringent hybridization conditions. Antibodies of the present invention may also be described or specified in terms of their binding affinity to a polypeptide of the invention. Preferred binding affinities include those with an equilibrium dissociation constant or K_{D} from 10⁻⁸ to 10⁻¹⁵ M, 10⁻⁸ to 10⁻¹² M, 10⁻⁸ to 10⁻¹⁰ M, or 10⁻¹⁰ to 10⁻¹² M. The invention also provides antibodies that competitively inhibit binding of an antibody to an epitope of the invention as determined by any method known in the art for determining competitive binding, for example, the immunoassays described herein. In preferred embodiments, the antibody competitively inhibits binding to the epitope by at least 95%, at least 90%, at least 85%, at least 80%, at least 75%, at least 70%, at least 60%, or at least 50%.

### 5.5 VECTORS AND HOST CELLS

In another aspect, the present invention provides isolated nucleic acid sequences encoding an antibody as disclosed herein, vector constructs comprising a nucleotide sequence encoding the antibodies of the present invention, host cells comprising such a vector, and recombinant techniques for the production of the antibody.

For recombinant production of the antibody, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). Standard techniques for cloning and transformation may be used in the preparation of cell lines expressing the antibodies of the present invention.

### 5.5.1 VECTORS

Many vectors are available. The vector components generally include, but are not limited to, one or more of the following: a signal sequence, an origin of replication, one or more marker genes, an enhancer element, a promoter, and a transcription termination sequence. Recombinant expression vectors containing a nucleotide sequence encoding the antibodies of the present invention can be prepared using well known techniques. Expression vectors may include a nucleotide sequence operably linked to suitable transcriptional or translational regulatory nucleotide sequences such as those derived from mammalian, microbial, viral, or insect genes. Examples of regulatory sequences include transcriptional promoters, operators, enhancers. mRNA ribosomal binding sites, and/or other appropriate sequences which control transcription and translation initiation and termination. Nucleotide sequences are “operably linked” when the regulatory sequence functionally relates to the nucleotide sequence for the appropriate polypeptide. Thus, a promoter nucleotide sequence is operably linked to e.g., the antibody heavy chain sequence if the promoter nucleotide sequence controls the transcription of the appropriate nucleotide sequence. An example of a useful expression vector for expressing the antibodies of the present invention may be found in application WO 04/070011, which is incorporated herein by reference.

In addition, sequences encoding appropriate signal peptides that are not naturally associated with antibody heavy and/or light chain sequences can be incorporated into expression vectors. For example, a nucleotide sequence for a signal peptide (secretory leader) may be fused in-frame to the polypeptide sequence so that the antibody is secreted to the periplasmic space or into the medium. A signal peptide that is functional in the intended host cells enhances extracellular secretion of the appropriate antibody. The signal peptide may be cleaved from the polypeptide upon secretion of antibody from the cell. Examples of such secretory signals are well known and include, e.g., those described in U.S. Pat. Nos. 5,698,435; 5,698,417; and 6,204,023.

### 5.5.2 HOST CELLS

Host cells useful in the present invention are prokaryotic, yeast, or higher eukaryotic cells and include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g. Baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

Prokaryotes useful as host cells in the present invention include gram negative or gram positive organisms such as E. coli, B. subtilis, Enterobacter, Erwinia, Klebsiella, Proteus, Salmonella, Serratia, and Shigella, as well as Bacilli, Pseudomonas, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids such as the pKK223-3 (Pharmacia Fine Chemicals, Uppsala, Sweden), pGEM1 (Promega Biotec, Madison, Wis., USA), and the pET (Novagen, Madison, Wis., USA) and pRSET (Invitrogen Corporation, Carlsbad, Calif., USA) series of vectors (Studier, J Mol Biol 219:37 (1991); Schoepfer, Gene 124:83 (1993)). Promoter sequences commonly used for recombinant prokaryotic host cell expression vectors include T7, (Rosenberg, et al., Gene 56:125 (1987)), β-lactamase (penicillinase), lactose promoter system (Chang, et al., Nature 275:615 (1978); Goeddel, et al., Nature 281:544 (1979)), tryptophan (trp) promoter system (Goeddel, et al., Nucl Acids Res 8:4057 (1980)), and tac promoter (Sambrook, et al., Molecular Cloning, A Laboratory Manual, 2nd ed., Cold Spring Harbor Laboratory (1990)).

Yeasts or filamentous fungi useful in the present invention include those from the genus Saccharomyces, Pichia, Actinornycetes, Kluyveromyces, Schizosaccharomyces, Candida, Trichoderma, Neurospora, and filamentous fungi such as Neurospora, Penicillium, Tolypocladium, and Aspergillus. Yeast vectors will often contain an origin of replication sequence from a 2μ yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman, et al., J Biol Chem 255:2073 (1980)) or other glycolytic enzymes (Holland, et al., Biochem 17:4900 (1978)) such as enolase, glyeeraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other suitable vectors and promoters for use in yeast expression are further described in Fleer, et al., Gene 107:285 (1991). Other suitable promoters and vectors for yeast and yeast transformation protocols are well known in the art. Yeast transformation protocols are well known. One such protocol is described by Hinnen, et al., Proc Natl Acad Sci 75:1929 (1978). The Hinnen protocol selects for Trp⁺ transformants in a selective medium.

Mammalian or insect host cell culture systems may also be employed to express recombinant antibodies. In principle, any higher eukaryotic cell culture is workable, whether from vertebrate or invertebrate culture. Examples of invertebrate cells include plant and insect cells (Luckow, et al., Bio/Technology 6:47 (1988), Miller, et al., Genetics Engineering, Setlow, et al., eds. Vol. 8, pp. 277-9, Plenam Publishing (1986); Mseda, et al., Nature 315:592 (1985)). For example, Baculovirus systems may be used for production of heterologous proteins. In an insect system, Autographa californica nuclear polyhedrosis virus (AcNPV) may be used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter). Other hosts that have been identified include Aedes, Drosophila melanogaster, and Bombyx mori. A variety of viral strains for transfection are publicly available, e.g., the L-1 variant of AcNPV and the Bm-5 strain of Bombyx mori NPV, and such viruses may be used as the virus herein according to the present invention, particularly for transfection of Spodoptera frugiperda cells. Moreover, plant cells cultures of cotton, corn, potato, soybean, petunia, tomato, and tobacco and also be utilized as hosts.

Vertebrate cells, and propagation of vertebrate cells, in culture (tissue culture) has become a routine procedure. See Tissue Culture, Kruse, et al., eds., Academic Press (1973). Examples of useful mammalian host cell lines are monkey kidney; human embryonic kidney line; baby hamster kidney cells; Chinese hamster ovary cells/-DHFR (CHO, Urlaub, et al., Proc Acad Sci USA 77:4216 (1980)); mouse sertoli cells; human cervical carcinoma cells (HELA); canine kidney cells; human lung cells; human liver cells; mouse mammary tumor; and NS0 cells.

Host cells are transformed with the above-described vectors for antibody production and cultured in conventional nutrient media modified as appropriate for inducing promoters, transcriptional and translational control sequences, selecting transformants, or amplifying the genes encoding the desired sequences. Commonly used promoter sequences and enhancer sequences are derived from polyoma virus, Adenovirus 2, Simian virus 40 (SV40), and human cytomegalovirus (CMV). DNA sequences derived from the SV40 viral genome may be used to provide other genetic elements for expression of a structural gene sequence in a mammalian host cell. e.g., SV40 origin, early and late promoter, enhancer, splice, and polyadenylation sites. Viral early and late promoters are particularly useful because both are easily obtained from a viral genome as a fragment which may also contain a viral origin of replication. Exemplary expression vectors for use in mammalian host cells are commercially available.

The host cells used to produce the antibody of this invention may be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) are suitable for culturing host cells. In addition, any of the media described in Ham, et al., Meth Enzymol 58:44 (1979), Barnes, et al., Anal Biochem 102:255 (1980), and U.S. Pat. Nos. 4,767,704; 4,657,866; 4,560,655; 5,122,469; 5,712,163; or 6,048,728 may be used as culture media for the host cells. Any of these media may be supplemented as necessary with hormones and/or other growth factors (such as insulin, transferrin, or epidermal growth factor), salts (such as X-chlorides, where X is sodium, calcium, magnesium; and phosphates), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### 5.6 POLYNUCLEOTIDES ENCODING ANTIBODIES

The invention further provides polynucleotides or nucleic acids, e.g., DNA, comprising a nucleotide sequence encoding an antibody of the invention and fragments thereof. Exemplary polynucleotides include those encoding antibody chains comprising one or more of the amino acid sequences described herein. The invention also encompasses polynucleotides that hybridize under stringent or lower stringency hybridization conditions to polynucleotides that encode an antibody of the present invention.

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier, et al., Bio/Techniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A⁺ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence and corresponding amino acid sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook, et al., Molecular Cloning, A Laboratory Manual, 2nd ed., Cold Spring Harbor Laboratory (1990); Ausubel, et al., eds., Current Protocols in Molecular Biology, John Wiley & Sons (1998), which are both incorporated by reference herein in their entireties), to generate antibodies having a different amino acid sequence, for example to create amino acid substitutions, deletions, and/or insertions.

In a specific embodiment, the amino acid sequence of the heavy and/or light chain variable domains may be inspected to identify the sequences of the CDRs by well known methods, e.g. by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and preferably human framework regions (see, e.g., Chothia, et al., J Mol Biol 278: 457 (1998) for a listing of human framework regions). Preferably, the polynucleotide generated by the combination of the framework regions and CDRs encodes an antibody that specifically binds a polypeptide of the invention. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison, et al., Proc Natl Acad Sci 81:851 (1984); Neuberger, et al., Nature 312:604 (1984); Takeda, et al., Nature 314:452 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423 (1988); Huston, et al., Proc Natl Acad Sci USA 85:5879 (1988); and Ward, et al., Nature 334:544 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra, et al., Science 242:1038 (1988)).

### 5.7 METHODS OF PRODUCING ANTI-OX40 ANTIBODIES

The antibodies of the invention can be produced by any method known in the art for the synthesis of antibodies, in particular, by chemical synthesis or preferably, by recombinant expression techniques.

Recombinant expression of an antibody of the invention, or fragment, derivative, or analog thereof, (e.g., a heavy or light chain of an antibody of the invention or a single chain antibody of the invention), requires construction of an expression vector containing a polynucleotide that encodes the antibody or a fragment of the antibody. Once a polynucleotide encoding an antibody molecule has been obtained, the vector for the production of the antibody may be produced by recombinant DNA technology. An expression vector is constructed containing antibody coding sequences and appropriate transcriptional and translational control signals. These methods include, for example, in vitro recombinant DNA techniques, synthetic techniques, and in vivo genetic recombination.

The expression vector is transferred to a host cell by conventional techniques and the transfected cells are then cultured by conventional techniques to produce an antibody of the invention. In one aspect of the invention, vectors encoding both the heavy and light chains may be co-expressed in the host cell for expression of the entire immunoglobulin molecule, as detailed below.

A variety of host-expression vector systems may be utilized to express the antibody molecules of the invention as described above. Such host-expression systems represent vehicles by which the coding sequences of interest may be produced and subsequently purified, but also represent cells which may, when transformed or transfected with the appropriate nucleotide coding sequences, express an antibody molecule of the invention in situ. Bacterial cells such as E. coli, and eukaryotic cells are commonly used for the expression of a recombinant antibody molecule, especially for the expression of whole recombinant antibody molecule. For example, mammalian cells such as CHO, in conjunction with a vector such as the major intermediate early gene promoter element from human cytomegalovirus, are an effective expression system for antibodies (Foecking, et al., Gene 45:101 (1986); Cockett, et al., Bio/Technology 8:2 (1990)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include, but are not limited to, CHO, COS, 293, 3T3, or myeloma cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for one to two days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler, et al., Cell 11:223 (1977)), hypoxanthine-guanine phosphoribosyltransferase (Szybalska, et al., Proc Nail Acad Sci USA 48:202 (1992)), and adenine phosphoribosyltransferase (Lowy, et al., Cell 22:817 (1980)) genes can be employed in tk, hgprt or aprt-cells, respectively. Also, antimetabolite resistance can be used as the basis of selection for the following genes: dhfr, which confers resistance to methotrexate (Wigler, et al., Proc Nail Acad Sci USA 77:357 (1980); O'Hare, et al., Proc Natl Acad Sci USA 78:1527 (1981)); gpt, which confers resistance to mycophenolic acid (Mulligan, et al., Proc Nail Acad Sci USA 78:2072 (1981)); neo, which confers resistance to the aminoglycoside G-418 (Wu, et al., Biotherapy 3:87 (1991)); and hygro, which confers resistance to hygromycin (Santerre, et al., Gene 30:147 (1984)). Methods commonly known in the art of recombinant DNA technology may be routinely applied to select the desired recombinant clone, and such methods are described, for example, in Ausubel, et al., eds., Current Protocols in Molecular Biology, John Wiley & Sons (1993); Kriegler, Gene Transfer and Expression, A Laboratory Manual, Stockton Press (1990); and in Chapters 12 and 13, Dracopoli, et al., eds, Current Protocols in Human Genetics, John Wiley & Sons (1994); Colberre-Garapin, et al., J Mol Biol 150:1 (1981), which are incorporated by reference herein in their entireties.

The expression levels of an antibody molecule can be increased by vector amplification (for a review, see Bebbington, et al., “The use of vectors based on gene amplification for the expression of cloned genes in mammalian cells,” DNA Cloning, Vol. 3. Academic Press (1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse, et al., Mol Cell Biol 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature 322:52 (1986); Kohler, Proc Natl Acad Sci USA 77:2197 (1980)). The coding sequences for the heavy and light chains may comprise cDNA or genomic DNA.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and size-exclusion chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The present invention encompasses antibodies recombinantly fused or chemically conjugated (including both covalently and non-covalently conjugations) to a polypeptide. Fused or conjugated antibodies of the present invention may be used for ease in purification. See e.g., PCT publication WO 93/21232; EP 439,095; Naramura, et al., Immunol Lett 39:91 (1994); U.S. Pat. No. 5,474,981; Gillies, et al., Proc Nail Acad Sci USA 89:1428 (1992); Fell, et al., J Immunol 146:2446 (1991), which are incorporated by reference in their entireties.

Moreover, the antibodies or fragments thereof of the present invention can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz, et al., Proc Natl Acad Sci USA 86:821 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson, et al., Cell 37:767 (1984)) and the “flag” tag.

### 5.8 ANTIBODY PURIFICATION

When using recombinant techniques, the antibody can be produced intracellularly, in the periplasmic space, or directly secreted into the medium. If the antibody is produced intracellularly, as a first step, the particulate debris, either host cells or lysed fragments, may be removed, for example, by centrifugation or ultrafiltration. Carter, et al., Bio/Technology 10:163 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 minutes. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human IgG1, IgG2 or IgG4 heavy chains (Lindmark, et al., J Immunol Meth 62:1 (1983)). Protein G is recommended for all mouse isotypes and for human IgG3 (Guss, et al., EMBO J5:1567 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABXTM resin (J. T. Baker; Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered. Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### 5.9 PHARMACEUTICAL FORMULATIONS

Therapeutic formulations of the polypeptide or antibody may be prepared for storage as lyophilized formulations or aqueous solutions by mixing the polypeptide having the desired degree of purity with optional “pharmaceutically-acceptable” carriers, excipients or stabilizers typically employed in the art (all of which are termed “excipients”), i.e., buffering agents, stabilizing agents, preservatives, isotonifiers, non-ionic detergents, antioxidants, and other miscellaneous additives. See Remington's Pharmaceutical Sciences, 16th edition, Osol, Ed. (1980). Such additives must be nontoxic to the recipients at the dosages and concentrations employed.

Buffering agents help to maintain the pH in the range which approximates physiological conditions. They are preferably present at concentration ranging from about 2 mM to about 50 mM. Suitable buffering agents for use with the present invention include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate butler (e.g., oxalic acid-sodium oxalate mixture, oxalic acid-sodium hydroxide mixture, oxalic acid-potassium oxalate mixture, etc.), lactate butlers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, there may be mentioned phosphate buffers, histidine buffers and trimethylamine salts such as Tris.

Preservatives may be added to retard microbial growth, and may be added in amounts ranging from 0.2%-1% (w/v). Suitable preservatives for use with the present invention include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” may be added to ensure isotonicity of liquid compositions of the present invention and include polhydric sugar alcohols, preferably trihydric or higher sugar alcohols, such as glycerin, erythritol, arabitol, xylitol, sorbitol and mannitol.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, alpha.-monothioglycerol and sodium thio sulfate; low molecular weight polypeptides (i.e. <10 residues); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccacharides such as raffinose; and polysaccharides such as dextran. Stabilizers may be present in the range from 0.1 to 10,000 weights per part of weight active protein.

Non-ionic surfactants or detergents (also known as “wetting agents”) may be added to help solubilize the therapeutic agent as well as to protect the therapeutic protein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), Pluronic polyols, polyoxyethylene sorbitan monoethers (TWEEN®-20, TWEEN®-80, etc.). Non-ionic surfactants may be present in a range of about 0.05 mg/ml to about 1.0 mg/ml, preferably about 0.07 mg/ml to about 0.2 mg/ml.

Additional miscellaneous excipients include bulking agents, (e.g., starch), chelating agents (e.g. EDTA), antioxidants (e.g., ascorbic acid, methionine, vitamin E), and cosolvents. The formulation herein may also contain more than one active compound as necessary for the particular indication being treated, preferably those with complementary activities that do not adversely affect each other. For example, it may be desirable to further provide an immunosuppressive agent. Such molecules are suitably present in combination in amounts that are effective for the purpose intended. The active ingredients may also be entrapped in microcapsule prepared, for example, by coascervation techniques or by interfacial polymerization, for example, hydroxymethylcellulose or gelatin-microcapsule and poly-(methylmethacylate) microcapsule, respectively, in colloidal drug delivery systems (for example, liposomes, albumin micropheres, microemulsions, nano-particles and nanocapsules) or in macroemulsions. Such techniques are disclosed in Remington's Pharmaceutical Sciences, 16th edition, Osal, Ed. (1980).

The formulations to be used for in vivo administration must be sterile. This is readily accomplished, for example, by filtration through sterile filtration membranes. Sustained-release preparations may be prepared. Suitable examples of sustained-release preparations include semi-permeable matrices of solid hydrophobic polymers containing the antibody, which matrices are in the form of shaped articles, e.g., films, or microcapsules. Examples of sustained-release matrices include polyesters, hydrogels (for example, poly/2-hydroxyethyl-methacrylate), poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C. resulting in a loss of biological activity and possible changes in immunogenicity.

Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The amount of therapeutic polypeptide, antibody, or fragment thereof which will be effective in the treatment of a particular disorder or condition will depend on the nature of the disorder or condition, and can be determined by standard clinical techniques. Where possible, it is desirable to determine the dose response curve and the pharmaceutical compositions of the invention first in vitro, and then in useful animal model systems prior to testing in humans.

In a preferred embodiment, an aqueous solution of therapeutic polypeptide, antibody or fragment thereof is administered by subcutaneous injection. Each dose may range from about 0.5 μg to about 50 μg per kilogram of body weight, or more preferably, from about 3 μg to about 30 μg per kilogram body weight.

The dosing schedule for subcutaneous administration may vary form once a month to daily depending on a number of clinical factors, including the type of disease, severity of disease, and the subject's sensitivity to the therapeutic agent.

### 5.10 DIAGNOSTIC USES FOR ANTI-OX40 ANTIBODIES

The antibodies of the invention include derivatives that are modified, i.e., by the covalent attachment of any type of molecule to the antibody, such that covalent attachment does not interfere with binding to OX40. For example, but not by way of limitation, the antibody derivatives include antibodies that have been modified, e.g., by biotinylation, HRP, or any other detectable moiety.

Antibodies of the present invention may be used, for example, but not limited to, to purify or detect OX40, including both in vitro and in vivo diagnostic methods. For example, the antibodies have use in immunoassays for qualitatively and quantitatively measuring levels of OX40 in biological samples. See, e.g., Harlow, et al., Antibodies: A Laboratory Manual, Cold Spring Harbor Laboratory Press, 2nd ed. (1988), which is incorporated by reference herein in its entirety.

As discussed in more detail below, the antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays.

The present invention further encompasses antibodies or fragments thereof conjugated to a diagnostic agent. The antibodies can be used diagnostically, for example, to detect expression of a target of interest in specific cells, tissues, or serum; or to monitor the development or progression of an immunologic response as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate (such as, for example, a linker known in the art) using techniques known in the art. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Techniques for conjugating enzymes to antibodies are described in O'Sullivan, et al., “Methods for the Preparation of Enzyme-Antibody Conjugates for Use in Enzyme Immunoassay,” in Methods in Enzymology, Langone, et al., eds. pp. 147-66, Academic Press (1981). See, for example, U.S. Pat. No. 4,741,900 for metal ions which can be conjugated to antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

Sometimes, the label is indirectly conjugated with the antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g. digloxin) and one of the different types of labels mentioned above is conjugated with an anti-hapten antibody (e.g. anti-digloxin antibody). Thus, indirect conjugation of the label with the antibody can be achieved.

In another embodiment of the invention, the antibody need not be labeled, and the presence thereof can be detected using a labeled antibody which binds to the antibody.

The antibodies of the present invention may be employed in any known assay method, such as competitive binding assays, direct and indirect sandwich assays, and immunoprecipitation assays. See Zola, Monoclonal Antibodies: A Manual of Techniques, pp. 147-158. CRC Press (1987).

Competitive binding assays rely on the ability of a labeled standard to compete with the test sample for binding with a limited amount of antibody. The amount of target in the test sample is inversely proportional to the amount of standard that becomes bound to the antibodies. To facilitate determining the amount of standard that becomes bound, the antibodies generally are insolubilized before or after the competition. As a result, the standard and test sample that are bound to the antibodies may conveniently be separated from the standard and test sample which remain unbound.

Sandwich assays involve the use of two antibodies, each capable of binding to a different immunogenic portion, or epitope, or the protein to be detected. In a sandwich assay, the test sample to be analyzed is bound by a first antibody which is immobilized on a solid support, and thereafter a second antibody binds to the test sample, thus forming an insoluble three-part complex. See e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

Antibodies may be attached to solid supports, which are particularly useful for immunoassays or purification of the target antigen. Such solid supports include, but are not limited to, glass, cellulose, polyacrylamide, nylon, polystyrene, polyvinyl chloride or polypropylene. In this process, the antibodies are immobilized on a solid support such as SEPHADEX™ resin or filter paper, using methods well known in the art. The immobilized antibodies are contacted with a sample containing the target to be purified, and thereafter the support is washed with a suitable solvent that will remove substantially all the material in the sample except the target to be purified, which is bound to the immobilized antibodies. Finally, the support is washed with another suitable solvent, such as glycine buffer, that will release the target from the antibodies.

Labeled antibodies, and derivatives and analogs thereof, which specifically bind to OX40, can be used for diagnostic purposes to detect, diagnose, or monitor diseases, disorders, and/or conditions associated with the aberrant expression and/or activity of OX40. The invention provides for the detection of aberrant expression of OX40, comprising (a) assaying the expression of OX40 in cells or body fluid of an individual using one or more antibodies of the present invention specific to OX40 and (b) comparing the level of gene expression with a standard gene expression level, whereby an increase or decrease in the assayed OX40 expression level compared to the standard expression level is indicative of aberrant expression.

Antibodies may be used for detecting the presence and/or levels of OX40 in a sample, e.g., a bodily fluid or tissue sample. The detecting method may comprise contacting the sample with an OX40 antibody and determining the amount of antibody that is bound to the sample. For immunohistochemistry, the sample may be fresh or frozen or may be embedded in paraffin and fixed with a preservative such as formalin, for example.

The invention provides a diagnostic assay for diagnosing a disorder, comprising (a) assaying the expression of OX40 in cells or body fluid of an individual using one or more antibodies of the present invention and (b) comparing the level of gene expression with a standard gene expression level, whereby an increase or decrease in the assayed gene expression level compared to the standard expression level is indicative of a particular disorder.

Antibodies of the invention can be used to assay protein levels in a biological sample using classical immunohistological methods known to those of skill in the art (e.g., see Jalkanen, et al., J Cell Biol 101:976 (1985); Jalkanen, et al., J Cell Biol 105:3087 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹³¹I, ¹²⁵I, ¹²¹I), carbon (¹⁴C), sulfur (³⁵S), tritium (³H), indium (¹¹²In, ¹¹¹In), and technetium (⁹⁹Tc); luminescent labels, such as luminol; and fluorescent labels, such as fluorescein, rhodamine, and biotin. Radioisotope-bound isotopes may be localized using immunoscintiography.

One aspect of the invention is the detection and diagnosis of a disease or disorder associated with aberrant expression of OX40 in an animal, preferably a mammal and most preferably a human. In one embodiment, diagnosis comprises: a) administering (for example, parenterally, subcutaneously, or intraperitoneally) to a subject an effective amount of a labeled molecule which specifically binds to OX40; b) waiting for a time interval following the administration permitting the labeled molecule to preferentially concentrate at sites in the subject where the polypeptide is expressed (and for unbound labeled molecule to be cleared to background level); c) determining background level; and d) detecting the labeled molecule in the subject, such that detection of labeled molecule above the background level indicates that the subject has a particular disease or disorder associated with aberrant expression of OX40. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹Tc. The labeled antibody or antibody fragment will then preferentially accumulate at the location of cells which contain the specific protein. In viva imaging is described in Burchiel, et al., “Immunopharmacokinetics of Radiolabeled Antibodies and Their Fragments.” Chapter 13 in Tumor Imaging: The Radiochemical Detection of Cancer, Burchiel, et al., eds., Masson Publishing (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours, 6 to 24 hours, or 6 to 12 hours. In another embodiment the time interval following administration is 5 to 20 days or 5 to 10 days.

In an embodiment, monitoring of the disease or disorder is carried out by repeating the method for diagnosing the disease or disease, for example, one month after initial diagnosis, six months after initial diagnosis, one year after initial diagnosis, etc.

Presence of the labeled molecule can be detected in the patient using methods known in the art for in viva scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patent using positron emission-tomography. In yet another embodiment, the molecule is labeled with a paramagnetic label and is detected in a patient using magnetic resonance imaging (MRI).

In another aspect, the present invention provides a method for diagnosing the predisposition of a patient to develop diseases caused by the unregulated expression of cytokines. Increased amounts of OX40 in certain patient cells, tissues, or body fluids may indicate that the patient is predisposed to certain diseases. In one embodiment, the method comprises collecting a cell, tissue, or body fluid sample a subject known to have low or normal levels of OX40, analyzing the tissue or body fluid for the presence of OX40 in the tissue, and predicting the predisposition of the patient to certain diseases based upon the level of expression of OX40 in the tissue or body fluid. In another embodiment, the method comprises collecting a cell, tissue, or body fluid sample known to contain a defined level of OX40 from a patient, analyzing the tissue or body fluid for the amount of OX40, and predicting the predisposition of the patient to certain diseases based upon the change in the amount of OX40 compared to a defined or tested level established for normal cell, tissue, or bodily fluid. The defined level of OX40 may be a known amount based upon literature values or may be determined in advance by measuring the amount in normal cell, tissue, or body fluids. Specifically, determination of OX40 levels in certain tissues or body fluids permits specific and early, preferably before disease occurs, detection of diseases in the patient. Diseases that can be diagnosed using the present method include, but are not limited to, the diseases described herein. In the preferred embodiment, the tissue or body fluid is peripheral blood, peripheral blood leukocytes, biopsy tissues such as lung or skin biopsies, and tissue.

The antibody of the present invention can be provided in a kit, i.e., packaged combination of reagents in predetermined amounts with instructions for performing the diagnostic assay. Where the antibody is labeled with an enzyme, the kit may include substrates and cofactors required by the enzyme (e.g., a substrate precursor which provides the detectable chromophore or fluorophore). In addition, other additives may be included, such as stabilizers, buffers (e.g., a block buffer or lysis buffer), and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

### 5.11 THERAPEUTIC USES OF ANTI-OX40 ANTIBODIES

It is contemplated that the antibodies of the present invention may be used to treat a mammal. In one embodiment, the antibody is administered to a nonhuman mammal for the purposes of obtaining preclinical data, for example. Exemplary nonhuman mammals to be treated include nonhuman primates, dogs, cats, rodents and other mammals in which preclinical studies are performed. Such mammals may be established animal models for a disease to be treated with the antibody or may be used to study toxicity of the antibody of interest. In each of these embodiments, dose escalation studies may be performed on the mammal.

An antibody, with or without a therapeutic moiety conjugated to it, administered alone or in combination with cytotoxic factor(s) can be used as a therapeutic. The present invention is directed to antibody-based therapies which involve administering antibodies of the invention to an animal, a mammal, or a human, for treating an OX40-mediated disease, disorder, or condition. The animal or subject may be an animal in need of a particular treatment, such as an animal having been diagnosed with a particular disorder, e.g., one relating to OX40. Antibodies directed against OX40 are useful for against allergy, asthma, autoimmune and inflammatory diseases in animals, including but not limited to cows, pigs, horses, chickens, cats, dogs, non-human primates etc., as well as humans. For example, by administering a therapeutically acceptable dose of an antibody, or antibodies, of the present invention, or a cocktail of the present antibodies, or in combination with other antibodies of varying sources, autoimmune or inflammatory disease symptoms may be reduced or eliminated in the treated mammal.

Therapeutic compounds of the invention include, but are not limited to, antibodies of the invention (including fragments, analogs and derivatives thereof as described herein) and nucleic acids encoding antibodies of the invention as described below (including fragments, analogs and derivatives thereof and anti-idiotypic antibodies as described herein). The antibodies of the invention can be used to treat, inhibit, or prevent diseases, disorders, or conditions associated with aberrant expression and/or activity of OX40, including, but not limited to, any one or more of the diseases, disorders, or conditions described herein. The treatment and/or prevention of diseases, disorders, or conditions associated with aberrant expression and/or activity of OX40 includes, but is not limited to, alleviating at least one symptoms associated with those diseases, disorders, or conditions. Antibodies of the invention may be provided in pharmaceutically acceptable compositions as known in the art or as described herein.

Anti-OX40 antibodies of the present invention may be used therapeutically in a variety of diseases. The present invention provides a method for preventing or treating OX40-mediated diseases in a mammal. The method comprises administering a disease preventing or treating amount of anti-OX40 antibody to the mammal. The anti-OX40 antibody binds to OX40 and regulates cytokine and cellular receptor expression resulting in cytokine levels characteristic of non-disease states. OX40 signaling has been linked to various diseases such as allergy, asthma, and diseases associated with autoimmunity and inflammation, which includes multiple sclerosis, rheumatoid arthritis, inflammatory bowel disease, graft-versus-host disease, experimental autoimmune encephalomyelitis (EAE), experimental leishmaniasis, collagen-induced arthritis, colitis (such as ulcerative colitis), contact hypersensitivity reactions, diabetes, Crohn's Disease, and Grave's Disease.

Antibodies of the present invention may also be useful to prevent and treat other diseases, including sarcoidosis, autoimmune ocular diseases, autoimmune uveitis, atopic dermatitis, myasthenia gravis, autoimmune neuropathies (such as Guillain-Barré), autoimmune ureitis, autoimmune hemolytic anemia, pernicious anemia, autoimmune thrombocytopenia, temporal artertis, anti-phospholipid syndrome, vasculitides (such as Wegener's granulomatosis), Behcet's disease, psoriasis, dermatitis herpetiformis, pemphigus vulgaris, vitiligo, primary biliary cirrhosis, autoimmune hepatitis, Hashimoto's thyroiditis, autoimmune oophoritis and orchitis, autoimmune disease of the adrenal gland, systemic lupus erythematosis, scleroderma, dermatomyositis, polymysitis, dermatomyositis, spondyloarthropathies (such as ankylosing spondylitis), Reiter's Syndrome, Sjogren's syndrome, and others.

A therapeutic agent for use in a host subject preferably elicits little to no immunogenic response against the agent in said subject. In one embodiment, the invention provides such an agent. For example, in one embodiment, the invention provides a humanized antibody that elicits and/or is expected to elicit a human anti-mouse antibody response (HAMA) at a substantially reduced level compared to an antibody comprising the heavy and light chain variable regions in a host subject. In another example, the invention provides a humanized antibody that elicits and/or is expected to elicit minimal or no human anti-mouse antibody response (HAMA). In one example, an antibody of the invention elicits anti-mouse antibody response that is at or less than a clinically-acceptable level.

The amount of the antibody which will be effective in the treatment, inhibition, and prevention of a disease or disorder associated with aberrant expression and/or activity of OX40 can be determined by standard clinical techniques. The dosage will depend on the type of disease to be treated, the severity and course of the disease, whether the antibody is administered for preventive or therapeutic purposes, previous therapy, the patient's clinical history and response to the antibody, and the discretion of the attending physician. The antibody can be administered in treatment regimes consistent with the disease, e.g., a single or a few doses over one to several days to ameliorate a disease state or periodic doses over an extended time to prevent allergy or asthma. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose response curves derived from in vitro or animal model test systems.

For antibodies, the dosage administered to a patient is typically 0.1 mg/kg to 150 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation. For repeated administrations over several days or longer, depending on the condition, the treatment is sustained until a desired suppression of disease symptoms occurs. However, other dosage regimens may be useful. The progress of this therapy is easily monitored by conventional techniques and assays. An exemplary dosing regimen for an anti-LFA-1 or anti-ICAM-1 antibody is disclosed in WO 94/04188.

The antibodies of the present invention, which may be in the form of a composition, should be formulated, dosed and administered in a manner consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody composition to be administered will be governed by such considerations, and is the minimum amount necessary to prevent, ameliorate, or treat a disease or disorder. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as used hereinbefore or about from 1 to 99% of the heretofore employed dosages.

The antibodies of this invention may be advantageously utilized in combination with other monoclonal or chimeric antibodies, or with lymphokines or hematopoietic growth factors (such as, e.g., IL-2, IL-3 IL-7, and IFN-γ), for example, which serve to increase the number or activity of effector cells which interact with the antibodies.

The antibodies of the invention may be administered alone or in combination with other types of treatments, such as anti-inflammatory therapies, immunosuppressive drugs, immunotherapy, chemotherapy, bronchodilators, anti-IgE molecules, anti-histamines, or anti-leukotrienes.

In a preferred aspect, the antibody is substantially purified (e.g., substantially free from substances that limit its effect or produce undesired side-effects). Various delivery systems are known and can be used to administer an antibody of the present invention, including injection, e.g., encapsulation in liposomes, microparticles, microcapsules, recombinant cells capable of expressing the compound, receptor-mediated endocytosis (see, e.g., Wu, et al., J Biol Chem 262:4429 (1987)), construction of a nucleic acid as part of a retroviral or, other vector, etc.

The anti-OX40 antibody can be administered to the mammal in any acceptable manner. Methods of introduction include but are not limited to parenteral, subcutaneous, intraperitoneal, intrapulmonary, intranasal, epidural, inhalation, and oral routes, and if desired for immunosuppressive treatment, intralesional administration. Parenteral infusions include intramuscular, intradermal, intravenous, intraarterial, or intraperitoneal administration. The antibodies or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the therapeutic antibodies or compositions of the invention into the central nervous system by any suitable route, including intraventricular and intrathecal injection: intraventricular injection may be facilitated by an intraventricular catheter, for example, attached to a reservoir, such as an Ommaya reservoir. In addition, the antibody is suitably administered by pulse infusion, particularly with declining doses of the antibody. Preferably the dosing is given by injections, most preferably intravenous or subcutaneous injections, depending in part on whether the administration is brief or chronic.

Pulmonary administration can also be employed. e.g., by use of an inhaler or nebulizer, and formulation with an aerosolizing agent. The antibody may also be administered into the lungs of a patient in the form of a dry powder composition (See e.g., U.S. Pat. No. 6,514,496).

In a specific embodiment, it may be desirable to administer the therapeutic antibodies or compositions of the invention locally to the area in need of treatment; this may be achieved by, for example, and not by way of limitation, local infusion, topical application, by injection, by means of a catheter, by means of a suppository, or by means of an implant, said implant being of a porous, non-porous, or gelatinous material, including membranes, such as sialastic membranes, or fibers. Preferably, when administering an antibody of the invention, care must be taken to use materials to which the protein does not absorb.

In another embodiment, the antibody can be delivered in a vesicle, in particular a liposome (see Langer, Science 249:1527 (1990); Treat, et al., in Liposomes in the Therapy of Infectious Disease and Cancer, Lopez-Berestein, et al., eds., pp. 353-365 (1989); Lopez-Berestein, ibid., pp. 317-27; see generally ibid.).

In yet another embodiment, the antibody can be delivered in a controlled release system. In one embodiment, a pump may be used (see Langer, Science 249:1527 (1990); Sefton, CRC Crit Ref Biomed Eng 14:201 (1987); Buchwald, et al., Surgery 88:507 (1980); Saudek, et al., N Engl J Med 321:574 (1989)). In another embodiment, polymeric materials can be used (see Medical Applications of Controlled Release, Langer, et al., eds., CRC Press (1974); Controlled Drug Bioavailability, Drug Product Design and Performance, Smolen, et al., eds., Wiley (1984); Ranger, et al., J Macromol Sci Rev Macromol Chem 23:61 (1983); see also Levy, et al., Science 228:190 (1985); During, et al., Ann Neurol 25:351 (1989); Howard, et al., J Neurosurg 71:105 (1989)). In yet another embodiment, a controlled release system can be placed in proximity of the therapeutic target.

The present invention also provides pharmaceutical compositions. Such compositions comprise a therapeutically effective amount of the antibody and a physiologically acceptable carrier. In a specific embodiment, the term “physiologically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such physiological carriers can be sterile liquids, such as water and oils, including those of petroleum, animal, vegetable, or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable carriers are described in “Remington's Pharmaceutical Sciences” by E. W. Martin. Such compositions will contain an effective amount of the antibody, preferably in purified form, together with a suitable amount of carrier so as to provide the form for proper administration to the patient. The formulation should suit the mode of administration.

In one embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides a pharmaceutical pack or kit comprising one or more containers tilled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In addition, the antibodies of the present invention may be conjugated to various effector molecules such as heterologous polypeptides, drugs, radionucleotides, or toxins. See, e.g., PCT publications WO 92/08495: WO 91/14438; WO 89/12624; U.S. Pat. No. 5,314,995; and EP 396,387. An antibody or fragment thereof may be conjugated to a therapeutic moiety such as a cytotoxin (e.g., a cytostatic or cytocidal agent), a therapeutic agent, or a radioactive metal ion (e.g., alpha-emitters such as, for example, 213Bi). A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Techniques for conjugating such therapeutic moiety to antibodies are well known, see, e.g., Arnon, et al., “Monoclonal Antibodies For Immunotargeting Of Drugs In Cancer Therapy”, in Monoclonal Antibodies and Cancer Therapy, Reisfeld, et al. (eds.), pp. 243-56 Alan R. Liss (1985); Hellstrom, et al., “Antibodies For Drug Delivery”, in Controlled Drug Delivery, 2nd ed., Robinson, et al., eds., pp. 623-53, Marcel Dekker (1987); Thorpe, “Antibody Carriers Of Cytotoxic Agents In Cancer Therapy: A Review,” in Monoclonal Antibodies '84: Biological And Clinical Applications, Pinchera, et al., eds., pp. 475-506 (1985); “Analysis, Results. And Future Prospective Of The Therapeutic Use Of Radiolabeled Antibody In Cancer Therapy,” in Monoclonal Antibodies For Cancer Detection and Therapy, Baldwin, et al., eds., pp. 303-16. Academic Press (1985); and Thorpe, et al., Immunol Rev 62:119 (1982). Alternatively, an antibody can be conjugated to a second antibody to form an antibody heteroconjugate. See, e.g., U.S. Pat. No. 4,676,980.

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, an apoptotic agent, e.g., TNF-α. TNF-β, AIM I (See, International Publication No. WO 97/33899), AIM II (See, International Publication No. WO 97/34911), Fas Ligand (Takahashi, et al., Int Immunol, 6:1567 (1994)), VEGI (See, International Publication No. WO 99/23105); a thrombotic agent; an anti-angiogenic agent, e.g., angiostatin or endostatin; or biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

### 5.12 ANTIBODY-BASED GENE THERAPY

In a another aspect of the invention, nucleic acids comprising sequences encoding antibodies or functional derivatives thereof, are administered to treat, inhibit or prevent a disease or disorder associated with aberrant expression and/or activity of OX40, by way of gene therapy. Gene therapy refers to therapy performed by the administration to a subject of an expressed or expressible nucleic acid. In this embodiment of the invention, the nucleic acids produce their encoded protein that mediates a therapeutic effect. Any of the methods for gene therapy available can be used according to the present invention. Exemplary methods are described below.

For general reviews of the methods of gene therapy, see Goldspiel, et al., Clinical Pharmacy 12:488 (1993); Wu, et al., Biotherapy 3:87 (1991); Tolstoshev, Ann Rev Pharmacol Toxicol 32:573 (1993); Mulligan, Science 260:926 (1993); Morgan, et al., Ann Rev Biochem 62:191 (1993); May, TIBTECH 11:155 (1993).

In a one aspect, the compound comprises nucleic acid sequences encoding an antibody, said nucleic acid sequences being part of expression vectors that express the antibody or fragments or chimeric proteins or heavy or light chains thereof in a suitable host. In particular, such nucleic acid sequences have promoters operably linked to the antibody coding region, said promoter being inducible or constitutive, and, optionally, tissue-specific.

In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller, et al., Proc Natl Acad Sci USA 86:8932 (1989); Zijlstra, et al., Nature 342:435 (1989)). In specific embodiments, the expressed antibody molecule is a single chain antibody; alternatively, the nucleic acid sequences include sequences encoding both the heavy and light chains, or fragments thereof, of the antibody.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g. Wu, et al., J Biol Chem 262:4429 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller, et al., Proc Nail Acad Sci USA 86:8932 (1989); Zijlstra, et al., Nature 342:435 (1989)).

In a specific embodiment, viral vectors that contain nucleic acid sequences encoding an antibody of the invention are used. For example, a retroviral vector can be used (see Miller, et al., Meth Enzymol 217:581 (1993)). These retroviral vectors contain the components necessary for the correct packaging of the viral genome and integration into the host cell DNA. The nucleic acid sequences encoding the antibody to be used in gene therapy are cloned into one or more vectors, which facilitate the delivery of the gene into a patient. More detail about retroviral vectors can be found in Boesen, et al., Biotherapy 6:291 (1994), which describes the use of a retroviral vector to deliver the mdrl gene to hematopoietic stem cells in order to make the stem cells more resistant to chemotherapy. Other references illustrating the use of retroviral vectors in gene therapy are: Clowes, et al. J Clin Invest 93:644 (1994); Kiem, et al., Blood 83:1467 (1994); Salmons, et al., Human Gene Therapy 4:129 (1993); and Grossman, et al., Curr Opin Gen and Dev 3:110 (1993).

Adenoviruses may also be used in the present invention. Adenoviruses are especially attractive vehicles in the present invention for delivering antibodies to respiratory epithelia. Adenoviruses naturally infect respiratory epithelia. Other targets for adenovirus-based delivery systems are liver, the central nervous system, endothelial cells, and muscle. Adenoviruses have the advantage of being capable of infecting non-dividing cells. Kozarsky, et al., Curr Opin Gen Dev 3:499 (1993) present a review of adenovirus-based gene therapy. Bout, et al., Human Gene Therapy 5:3 (1994) demonstrated the use of adenovirus vectors to transfer genes to the respiratory epithelia of rhesus monkeys. Other instances of the use of adenoviruses in gene therapy can be found in Rosenfeld, et al., Science 252:431 (1991); Rosenfeld, et al., Cell 68:143 (1992); Mastrangeli, et al., J Clin Invest 91:225 (1993); PCT Publication WO94/12649; Wang, et al., Gene Therapy 2:775 (1995). Adeno-associated virus (AAV) has also been proposed for use in gene therapy (Walsh, et al., Proc Soc Exp Biol Med 204:289 (1993); U.S. Pat. Nos. 5,436,146; 6,632,670; and 6,642,051).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler, et al., Meth Enzymol 217:599 (1993); Cohen, et al., Meth Enzymol 217:618 (1993); Cline, Pharmac Ther 29:69 (1985)) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a one embodiment, the cell used for gene therapy is autologous to the patient. Nucleic acid sequences encoding an antibody of the present invention are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple, et al., Cell 71:973 (1992); Rheinwald, Meth Cell Bio 21A:229 (1980); Pittelkow, et al., Mayo Clinic Proc 61:771 (1986)).

### 6. EXAMPLES

This invention can be further illustrated by the following examples of preferred embodiments thereof, although it will be understood that these examples are included merely for purposes of illustration and are not intended to limit the scope of the invention unless otherwise specifically indicated.

### Example 1

### Preparation of a Human OX40/Fc Immunogen

Two forms of the OX40 receptor were used as immunogens to generate antagonist anti-OX40 antibodies of the present invention. The first was the soluble form of the human OX40 receptor expressed as fusion protein comprising human Fcγ1 and the extracellular domain encoded by nucleotide residues 105 to 627 of SEQ ID NO: 1. The second form was a cell-based immunogen comprising the full length OX40 expressed on the surface of stably transfected mouse fibroblast L-cells.

The soluble form of OX40 was cloned from an OX40 cDNA clone isolated from TCR-activated human CD4 T-cells. The two primers used to generate the OX40 cDNA clone were: (1) a sense primer having the nucleotide sequence cccaagcttaccccagcaacgaccggtgctgc (SEQ ID NO: 3) containing a HindIII site, and (2) an antisense primer having the nucleotide sequence cgcctcgaggacctccacgggccgggtgg (SEQ ID NO: 4) containing an XhoI site in frame with human Fcγ1. The resulting 540 bp PCR fragment was subcloned into a commercially available vector that contained a secretion signal peptide sequence at the 5′ end and a human Fcγ1 sequence (hinge and constant regions CH2 and CH3) at the 3′ end. The construct's composition was confirmed by sequencing.

For transient expression, OX40/Fcγ1 DNA was transfected into 293T-cells (Invitrogen, Carlsbad, Calif.) by Lipofectamine 2000 (Invitrogen, Carlsbad, Calif.) according to the manufacturer's protocol. At 72 hours post-transfection, culture supernatants from transfected cells were collected for purification. Stable expression of OX40/Fcγ1 was also established in a 293T-cell line. To confirm expression, culture supernatants were analyzed by sodium dodecyl sulfate polyacrylamide gel electrophoresis (SDS-PAGE). The separated proteins were transferred to nitrocellulose membrane and detected with horseradish peroxidase (HRP) conjugated mouse anti-human IgG (Fc) monoclonal antibody (Sigma, St. Louis, Mo.) or polyclonal goat anti-OX40 antibodies (R&D Systems, Minneapolis, Minn.) detected with HRP-donkey anti-goat IgG (Jackson ImmunoResearch Laboratories, West Grove, Pa.). The immunoreactive proteins were identified on film using enhanced chemo-luminescence detection (Pierce, Rockford, Ill.). OX40/Fcγ1 was purified with a protein-A affinity column (Invitrogen, Carlsbad, Calif.) equilibrated in phosphate-buffered saline (PBS). After applying the cell culture supernatant to the column, the resin was washed with approximately 20 column volumes of PBS followed by an SCC buffer wash (0.05 M sodium citrate, 0.5 M sodium chloride, pH 6.01 to remove unbound proteins. The OX40 fusion proteins were eluted using 0.05 M sodium citrate, 0.15 M sodium chloride (pH 3.0) followed by dialysis in PBS (pH 7.0). Fractions from the affinity column containing OX40/Fcγ1 were analyzed by SDS-PAGE. The purity of the proteins was analyzed by Coomassie Blue staining and the identity of the proteins by Western immunoblotting using goat anti-human IgG (Fc) antibody (Sigma, St Louis, Mo.) and goat anti-human OX40 antibody (R&D Systems, Minneapolis, Minn.) as described above.

The second form of immunogen was constructed using full length human OX40 cDNA that was cloned from TCR-activated human CD4 T-cells. The two primers used were OX40-specific primers having the nucleotide sequence caccatgtgcgtgggggctcggcggctggg (SEQ ID NO: 5), and gatcttggccagggtggagtgggcgtcggcc (SEQ ID NO: 6). The resulting PCR product was cloned directly into a commercially available vector and stably transfected into mouse L fibroblasts for the expression of high level of OX40. Prior to administration, the cell-based immunogen L-cells were irradiated with 6800 Grays using GammaCell 1000 Elite model A (MDS Nordion, Ottawa Canada) to inhibit proliferation of the immunogen.

### Example 2

### Generation of Anti-OX40 mAbs

Approximately 20 μg of the soluble OX40/Fcγ1 immunogen was combined with Freund's adjuvant (1:1) and administered to two six-week-old A/J mice (Harlan, Houston, Tex.) in three subcutaneous injections followed by one intraperitoneal injection (without adjuvant) at 7-day intervals. Three days after the final injection, the splenocytes of immunized mice were fused with murine myeloma SP2/0 cells according to the method of Groth and Scheidegger (J Immunol Methods, 1980) as described below.

For the cell-based immunogen, 3×10⁶ gamma-irradiated OX40-stably transfected L-cells were administered to six-week-old A/J mice (Harlan, Houston, Tex.) by three subcutaneous injections in PBS followed by one intraperitoneal injection at 7-day intervals. Three days after the final injection, the splenocytes of immunized mice were fused with murine myeloma SP2/0 cells as described below.

In the fusion leading to the generation of the anti-OX40 mAb, single cell suspensions were prepared from the spleens of immunized mice and used for fusion with immortalized Sp2/0 myeloma cells (at the ratio 1:1) using a suitable fusion agent, polyethylene glycol (M.W. 1450) (Kodak, Rochester, N.Y.) and 5% dimethylsulfoxide (Sigma, St Louis, Mo.) to form a hybridoma cells (Goding, Monoclonal Antibodies: Principles and Practice, Academic Press, pp. 59-103 (1986). After 10 days, the hybridoma cell lines were cultured in suitable DMEM culture complete-medium (Invitrogen, Carlsbad, Calif.), that preferably contained hypoxanthine-aminopterin-thymidine (HAT), a substance that inhibits the growth or survival of the unfused, immortalized cells. From 2 fusion-experiments 24,000 preferred immortalized cell lines were obtained that fused efficiently, supported stable, high-level production of antibody, and were sensitive to HAT medium.

### Example 3

### Screening for Antagonist Anti-OX-40 Antibodies

The culture medium in which the hybridoma cells were grown was assayed for the production of monoclonal antibodies (mAbs) directed against OX40. The binding specificity of mAbs produced by the hybridoma cells was determined by three different approaches: FMAT, ELISA, and flow cytometry immunoassay.

A. FMAT Screening

Using the FMAT approach, 220 hybridoma cell lines secreting mouse anti-human OX40 mAbs were identified by their strong reactivity with OX40-transiently transfected L-cells, but not with mock untransfected L-cells. Anti-OX40 hybridoma supernatants were screened using FMAT employing a macroconfocal scanning platform to image and quantify both cell number and fluorescence in a 96-well microtiter plate format. L-cells expressing OX40 antigen were incubated with 5 μl of anti-OX40 hybridoma supernatant and fluorescently-labeled with Cy-5-conjugated goat anti-mouse IgG-Fcγ antibodies (Jackson Laboratories, PA) that were diluted to 0.125 μg/ml in screening buffer. The plates were scanned after 2 hours of incubation using an FMAT 8100 HTS system (Applied Biosystem, CA). The results in Table 1 below show that clones B24 and B39 have comparable activity with the positive control.

B. ELISA Screening

Polyvinyl chloride 96-well plates (Nunc, Roskilde, Denmark) were coated overnight at 4° C. with OX40/Fcγ1 (5 ng/well) or with human Fcγ1 (25 ng/well) as a screening control, followed by a one our incubation with 2% BSA in PBS to block non-specific binding. Approximately 50 μl of hybridoma supernatant was added and incubated for 1 hour. After four washes with 0.05% TWEEN®-PBS, approximately 0.2 μg of HRP-goat anti-mouse IgG Fcγ (Jackson ImmunoResearch Laboratories, PA) was added for 1 hour at RT to detect the presence of bound anti-OX40 antibody. 100 μl of 1 mM TMB (Sigma) freshly made substrate solution was added to each well for 30 minutes and the reaction was stopped by adding 50 ml of 0.2M H₂SO₄ and the plates were read at 450 nm using a microplate reader (Molecular Devices, CA). Out of 24,000 wells, 220 were considered positive by FMAT and ELISA. Representative data is reported in Table 1.

TABLE 1

| | FL1 | Number of | |
|---|---|---|---|
| | (Mean Fluorescence) | Fluorescent Cells | OD₄₅₀ |
| Clone#B24 | 5560 | 39 | 3.4 |
| Clone#B39 | 7665.3 | 44 | 3.6 |
| Negative Control | 0 | 0 | NA |
| Positive Control | 5028.5 | 41 | NA |

C. Flow Cytometry Screening

The 220 positive hybridomas selected by FMAT and ELISA were also tested by flow cytometry using OX40 stably transfected L-cells that were stained using 5 μl of anti-OX40 hybridoma supernatants and fluorescently labeled by FITC-conjugated goat anti mouse IgG-Fcγ antibody (Jackson Laboratories, PA). The data presented in Table 2 corresponds to the percentage of total OX40-transfected L-cells stained using anti-OX40 hybridoma supernatant.

TABLE 2

| | Clone# B2 | | | |
|---|---|---|---|---|
| | % | Clone# B17 | Clone# B36 | Clone# B37 |
| | Fluorescent | % Fluorescent | % Fluorescent | % Fluorescent |
| | Cells | Cells | Cells | Cells |
| L-cells | 0.82 | 0.7 | 0.9 | 0.4 |
| OX40L | 30.4 | 0.3 | 15.0 | 32.7 |
| L-cells | | | | |

From the 220 anti-OX40 mAbs identified in Tables 1 and 2, the anti-OX40 mAbs clones B2, B24, B36, B37, and B39 showed positive binding to OX40L L-cells but not to untransfected L-cells. B-17 was rejected because it did not show binding to OX40L L-cells.

### Example 4

### Initial Characterization of Antagonist Anti-OX40 mAbs

After identifying the 220 hybridomas that produce antibodies specific to human OX40, the clones were subcloned by limiting dilution procedures and grown by standard methods (Goding, Monoclonal Antibodies: Principles and Practice, Academic Press, pp. 59-103 (1986)). The 220 anti-OX40 mAb secreted by the subclones were purified from the culture medium by conventional purification procedures using protein A-Sepharose.

A. Cell Line Preparation

The influence of OX40 costimulation on human CD4+ T-cell development was examined using specific cells lines and fresh isolated human naïve C D4+ T-cells to characterize the 220 anti-OX40 mAbs binders These cell lines included: (1) OX40 transfected human Hut-78 leukemic CD4+ T-cell line, (2) OX40L-transfected L fibroblast mouse L-cells and their parental cell lines (provided by Dr. Yong-Jun Liu, M.D. Anderson Cancer Center, Houston, Tex.), (3) human naïve CD4+ T-cells, and (4) human dendritic cells (DC) cultured in the presence of human IL-4 and GM-CSF and differentiated from isolated CD14+ monocytes. The buffy coat fraction of blood collected from healthy adult individuals served as a source of human naïve T-cells isolated by magnetic depletion (CD4+, CD45RA+, CD45RO− and CD14− phenotype); as a source of CD11c⁺ dendritic cells (DCs) isolated by cell sorting; or as a source of isolated CD14+ monocytes that were differentiated into immature DCs.

B. Functional Assays

1. Hut-78 Assay

OX40 transfected human Hut-78 leukemic CD4⁺ T-cells were primed for 48 hours in 96 well plates with 5 μg/ml anti-CD3 (OKT3, BD Bioscences, CA) and 1 μg/ml anti-CD28 mAbs (CD28.2, BD Bioscences, CA) in the presence of irradiated (6800 cGray) OX40L-transfected L-cells or untransfected L-cells at a ratio of 4:1 (OX40-Hut78 cells/L-cells). The 220 anti-OX40 mAbs identified in Example 3 were added at the beginning of the assay to screen for antagonistic activity defined as inhibition of CD4+ T-cell cytokine release. The assay was repeated three times using the same protocol. The experiment summarized in Table 3 is an example of three representative anti-OX40 mAbs (B66, B76, and B86) and showed that T-cells activated in the presence of OX40L transfected cells produced at least 10 times more IL-2 (138 pg/ml) and IL-13 (620 pg/ml) than did those activated in the presence of untransfected L-cells (22 pg/ml for IL-2 and 70 pg/ml for IL-13 production). The addition of purified anti-OX40 mAbs B66 and B76 to the cell culture blocked Hut-78 CD4⁺ T-cell line cytokine production in a dose responsive manner. Clone B66 inhibited 88% of IL-2 and 92% of IL-13 cytokine production at the highest concentration (Table 3). From the 220 anti-OX40 mAb tested, 10 clones showing more than 60% inhibition of IL-2 and IL-13 cytokine production were selected (data not shown).

TABLE 3

| | | | OX40-Hut-78+ + | | | | | | | | | | | | | |
|---|---|---|---|---|---|---|---|---|---|---|---|---|---|---|---|---|
| | | | OX40L-Lcells+ | | | | | | | | | | | | | |
| Cytokine | OX40 | OX40 | IC | | B66 | | | | B76 | | | | B86 | | | |
| Production | Hut- | Hut78+ + | (n: 3) | | (n: 3) | | | | (n: 3) | | | | (n: 3) | | | |
| AbConc (μg/ml) | 78+ | L-Cells | 0 | 20 | 0 | 1 | 5 | 20 | 0 | 1 | 5 | 20 | 0 | 1 | 5 | 20 |
| IL2 (pg/ml) ± | 22 | 0 | 138 | 148 | 138 | 61 | 27 | 15 | 138 | 69 | 21 | 12 | 138 | 161 | 135 | 152 |
| SD | 8.7 | 5.2 | 13.9 | 13.5 | 13.9 | 1.1 | 3 | 5.7 | 13.9 | 10.1 | 8.6 | 9.3 | 13.9 | 15 | 6.3 | 26.8 |
| IL13 (pg/ml) ± | 70 | 0 | 620 | 712 | 620 | 139 | 95 | 41 | 620 | 316 | 234 | 223 | 620 | 623 | 616 | 645 |
| SD | 0 | 18.7 | 49.5 | 64.7 | 49.5 | 33.3 | 21.1 | 23.6 | 49.5 | 11.3 | 25.1 | 46.2 | 49.5 | 22.8 | 27.9 | 17.1 |

C. Naïve CD4+ T-cell Assay

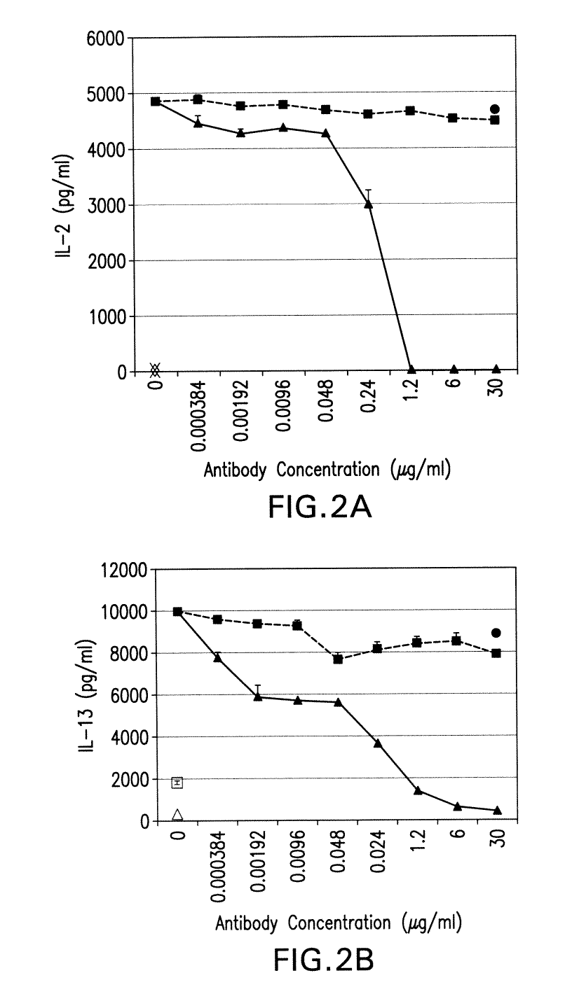

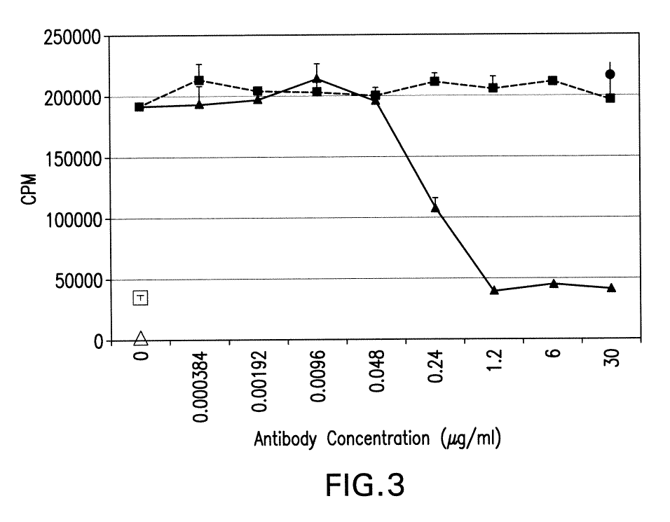

In order to block OX40 costimulation of T-cell proliferation and cytokine production, the 220 anti-OX40 mAbs were functionally screened for their ability to inhibit cytokine production, proliferation, and to induce apoptosis in human CD4+ T-cells. Naïve human CD4⁺ T-cells were isolated from the bully coat of human blood from healthy adult volunteers (Gulf Coast Regional Blood Center, Houston, Tex.) using a commercially available naïve CD4⁺ T-cell isolation kit (Miltenyi Biotec, Auburn, Calif.). The isolated naïve human CD4⁺ T-cells were activated with 5 μg/mL anti-CD3 (OKT3, BD Biosciences, CA) and 1 μg/ml anti-CD28 mAbs (CD28.2, BD Biosciences, CA) in the presence of irradiated OX40L-transfected L-cells or parental L-cells at the ratio of 1:4 (L-cells/naïve CD4⁺ T-cells). After seven days of culture, naïve CD4⁺ T-cells activated in the presence of OX40L transfectants produced at least 10 times more IL-2 and IL-13 than did those primed in the presence of L-cells (FIG. 2). Levels of cytokine production were measured using an ELISA-kit from R&D Systems (Minneapolis, Minn.). The cells also proliferated much more in the presence of OX40 co-stimulation as shown in FIG. 3). For FIGS. 2 and 3: the Open Square represents naïve CD4; the Open Triangle represents naïve CD4+ with control L-cells; Solid Triangles represent B66 with naïve CD4+ and OX40L L-cells; Solid Squares represent 2C4 (Isotype Control) with naïve CD4+ and OX40L L-cells; and Solid Circles represent G3-519 irrelevant mAb control with naïve CD4+ and OX40L-L-cells.

Addition of anti-OX40 mAb B66 to the culture inhibited cytokine release (FIG. 2), cell proliferation (FIG. 3), and induced apoptosis in a dose dependent manner in primed CD4⁺ T-cells as compared to 2C4 or G3-519 controls (Table 4).

TABLE 4

| | Apoptotic | Inhibition of |
|---|---|---|
| | cells (%) | survival (%) |
| Naïve CD4+ T-cells + L-cells | 21.9 | 22 |
| Naïve CD4+ T-cells + OX40L L-cells | 11.0 | 11 |
| Naïve CD4+ T-cells + OX40L L-cells + | 37.9 | 38 |
| B66 (20 μg/ml) | | |
| Naïve CD4+ T-cells + OX40L L-cells + | 32.3 | 32 |
| A10 (20 μg/ml) | | |
| Naïve CD4+ T-cells + OX40L L-cells + IC: | 11.7 | 12 |
| G3-519 (20 μg/ml) | | |

### Example 5

### Further Characterization of Selected Antagonist Anti-OX40 mAbs

Cytokines can control the differentiation of naïve CD4⁺ T-cells creating subsets of T-cells that are defined by their cytokine production profile. IL-12 drives the differentiation of Th1 cells which mainly produce IFN-γ and IL-2, and are responsible for controlling cell-mediated immunity and pro-inflammatory responses. IL-4 polarizes naïve CD4⁺ T-cells towards Th2 cells which mainly produce IL-4 and IL-5 and are responsible for the control of humoral immunity and anti-inflammatory responses. Two anti-OX40 mAbs, A10 and B66 selected from the initial characterization were tested in both Th1 and Th2 cells for their ability to block proliferation survival and cytokine release. To do so they were first converted to a chimeric form comprising the variable regions of the murine parent antibody and human constant regions. All commercial antibodies and cytokines were purchased from BD Biosciences, CA.

A. Th1 Assay

Human naïve CD4⁺ T-cells were isolated from the buffy coat of human blood using a kit as described in Example 4 (B)(2) above. The cells were primed for 7 days to induce a Th1-cytokine producing profile using 5 μg/ml anti-CD3 (OKT3, 1 μg/ml anti-CD28 (CD28.2), and 10 μg/ml anti-IL-4 neutralizing antibody (MP4-25D2) in medium supplemented with 50 ng/ml IL-12 cytokine and. The cells were cultured with anti-CD3 and anti-CD28 mAbs in the presence of irradiated OX40L-transfected L-cells or parental L-cells at the ratio 1:4 (L-cells/naïve CD4⁺ T-cells). The OX40 costimulation increases the survival of Th1 effector cells as shown by a decrease in the number of CD4⁺ cells positive for annexin staining (Table 5). The addition of chimeric anti-OX40 mAb B66 at the beginning of the co-culture of irradiated L-cells inhibited this survival. OX40 costimulation also increases IFN-γ production and the addition of B66 inhibited its production in a dose dependent manner (Table 6).

TABLE 5

| | Th1 |
|---|---|
| | Survival (%) |
| Naïve CD4⁺ T-cells + L-cells | 52.6 |
| Naïve CD4⁺ T-cells + OX40L L-cells | 65.9 |
| Naïve CD4⁺ T-cells + OX40L L-cells + B66 (0.2 μg/ml) | 58.4 |
| Naïve CD4⁺ T-cells + OX40L L-cells + B66 (2 μg/ml) | 55.9 |
| Naïve CD4⁺ T-cells + OX40L L-cells + B66 (20 μg/ml) | 53.2 |

TABLE 6

| | IFN-γ Conc | |
|---|---|---|
| | (pg/ml) | SD (n: 3) |
| Th1 cells | 10280.2 | 123.0 |
| Th1 cells + L-cells | 10934.4 | 267.7 |
| Th1 cells + OX40L L-cells | 33040.4 | 237.5 |
| Th1 cells + OX40L L-cells + B66 (0.2 μg/ml) | 17148.3 | 234.6 |
| Th1 cells + OX40L L-cells + B66 (2 μg/ml) | 11049.3 | 141.7 |
| Th1 cells + OX40L L-cells + B66 (20 μg/ml) | 11540.6 | 51.6 |

B. Th2 Assay

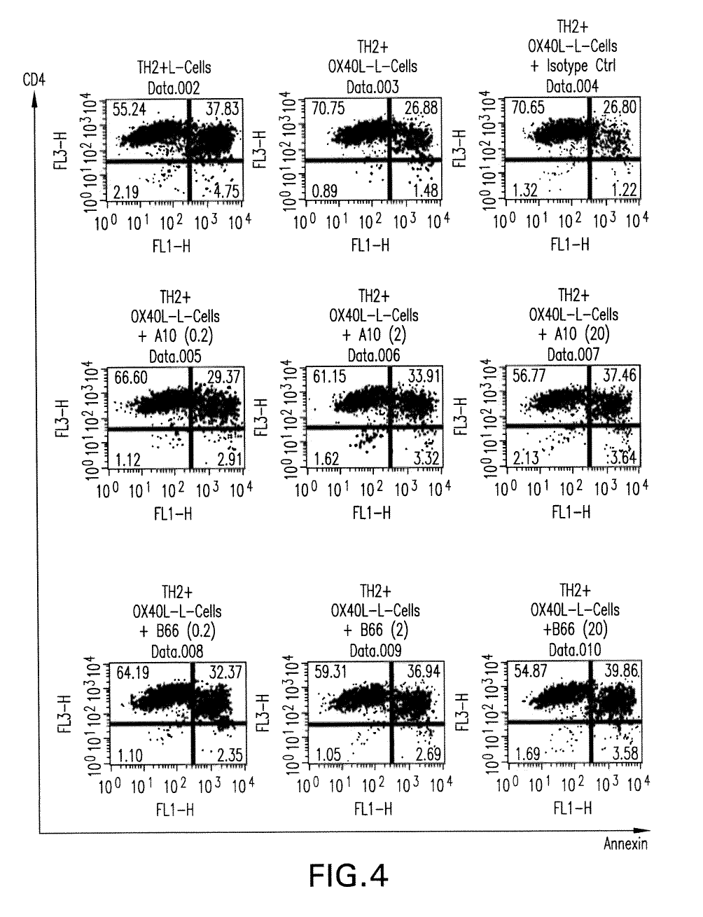

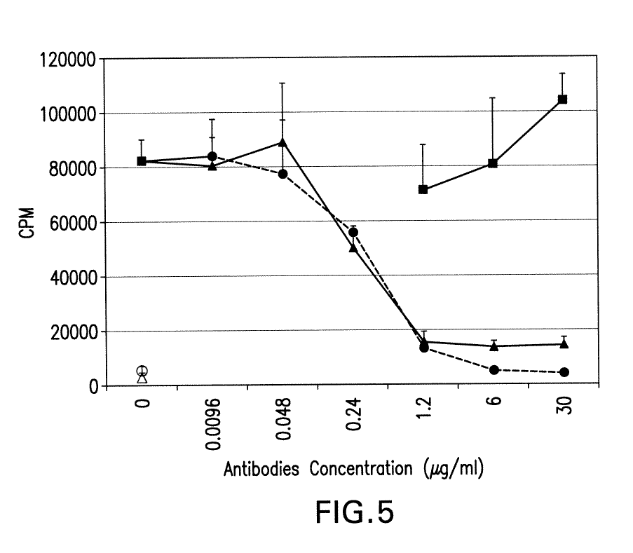

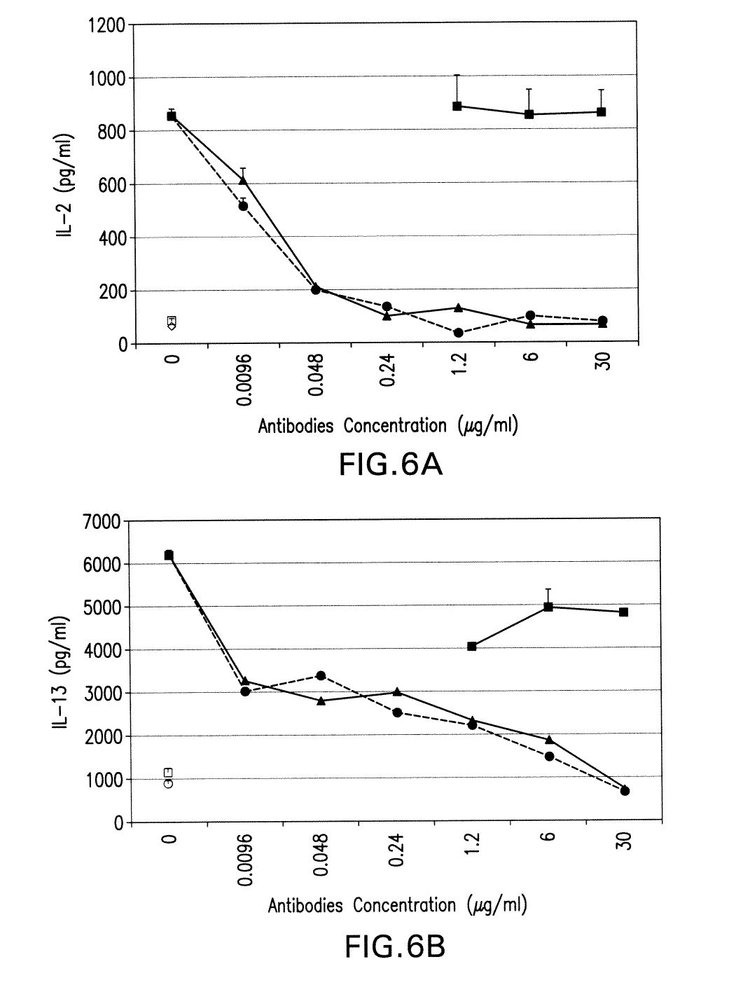

Human naïve CD4⁺ T-cells were isolated as described above. The cells were primed for 7 days to induce a Th2-cytokine producing profile using 5 μg/ml anti-CD3 1 μg/ml anti-CD28 and 10 μg/ml anti-IFN-γ neutralizing antibody in medium supplemented with 50 ng/ml IL-4 cytokine. The cells were cultured with anti-CD3 and anti-CD28 mAbs in the presence of irradiated OX40L-transfected L-cells or parental L-cells at the ratio 1:4 (L-cells/naïve CD4⁺T-cells). The OX40 costimulation increases the survival of Th2 effector cells as shown by a decrease in the number of CD4⁺ cells positive for annexin staining (FIG. 4). The addition of anti-OX40 mAbs A10 and B66 at the beginning of the co-culture of irradiated L-cells inhibited this survival (FIG. 4), as well as the proliferation of Th2 cells (FIG. 5) and cytokine production of IL-2 (FIG. 6A and IL-13 (FIG. 6B) in a dose dependent manner.

C. Th17 Assay

A unique subset of T-cells primed by the presence of IL-23 is characterized by the production of IL-17 and crucially involved in the pathogenesis of certain chronic inflammatory diseases. Recent data has demonstrated that Th17 cells represent a completely separate lineage of CD4⁺ T-cells that diverge early from Th1 and Th2 lineages and is antagonized by the cytokines and signaling pathways that govern the development of Th1 and Th2 cells. Th17 T-cells were primed to study the effect of OX40 costimulation and the ability of the antagonistic anti-OX40 mAbs to inhibit Th17 differentiation.

Naïve CD4⁺ T-cells were isolated from buffy coat as described in Example 4(B)(2). These cells were primed for 7 days in the presence of 100 ng/ml of IL-23 cytokine (R&D, Minneapolis, Minn.), 20 ng/ml each of hIL-6 and hTGFβ1, 5 μg/ml of pre-coated anti-CD3 (OKT3, BD Biosciences, CA), 1 μg/ml of soluble anti-CD28 (CD28.2), 10 μg/ml each of anti-IL4 (MP4-25D2) and anti-IFN-γ (B27). The primed cells were washed and sub-cultured for an additional 3 days with anti-CD3 and anti-CD28 mAbs in the presence of irradiated OX40L-transfected L-cells or parental L-cells at the ratio 1:4 (L-cells/naïve CD4⁺ T-cells). By inducing OX40 costimulation through OX40L-L-cells, Th17 cells proliferate 4-fold as compared to the same cells cultured in the absence of OX40L costimulation. Anti-OX40 mAbs A10 and B66 inhibited Th17 proliferation in a dose dependent manner (Table 7)).

TABLE 7

| | CMP Mean | |
|---|---|---|
| | (n: 3) | ±SD |
| Th17 cells + L-cells | 18092 | 2592.6 |
| Th17 cells + OX40L L-cells | 66035.7 | 2106.2 |
| Th17 cells + OX40L L-cells + Isotype | 70176.7 | 3080.8 |
| Control (20 μg/ml) | | |
| Th17 cells + OX40L L-cells + A10 (0.2 μg/ml) | 70176.7 | 7622.6 |
| Th17 cells + OX40L L-cells + A10 (2 μg/ml) | 40347.7 | 8059.5 |
| Th17 cells + OX40L L-cells + A10 (20 μg/ml) | 17456.7 | 563.9 |
| Th17 cells + OX40L L-cells + B66 (0.2 μg/ml) | 46735.3 | 10667.1 |
| Th17 cells + OX40L L-cells + B66 (2 μg/ml) | 31658.0 | 5492.6 |
| Th17 cells + OX40L L-cells + B66 (20 μg/ml) | 18336.3 | 956.4 |

Table 8 data demonstrated that OX40L significantly enhanced Th-17 survival (68% vs. 84%). This enhanced survival was inhibited by anti-OX40 mAbs A10 and B66 in dose dependent manner (Table 8).

TABLE 8

| | Th-17 |
|---|---|
| | Survival (%) |
| Th17 cells + L-cells | 68.4 |
| Th17 cells + OX40L L-cells | 84.3 |
| Th17 cells + OX40L L-cells + Isotype Control (20 μg/ml) | 84.3 |
| Th17 cells + OX40L L-cells + A10 (0.2 μg/ml) | 80.7 |
| Th17 cells + OX40L L-cells + A10 (2 μg/ml) | 72.3 |
| Th17 cells + OX40L L-cells + A10 (20 μg/ml) | 70.7 |
| Th17 cells + OX40L L-cells + B66 (0.2 μg/ml) | 77.0 |
| Th17 cells + OX40L L-cells + B66 (2 μg/ml) | 74.1 |
| Th17 cells + OX40L L-cells + B66 (20 μg/ml) | 67.3 |

By evaluating the intracellular expression of the protein Survivin, which is an inhibitor of apoptosis and a potential target of OX40 protein, the OX40 stimulation enhanced its expression. This enhanced expression was inhibited by anti-OX40 mAbs A10 and B66 in a dose dependent manner (Table 9).

TABLE 9

| | Survivin |
|---|---|
| | expression (%) |
| Th17 cells + L-cells | 19.2 |
| Th17 cells + OX40L L-cells | 49.0 |
| Th17 cells + OX40L L-cells + Isotype | 45.4 |
| Control (20 μg/ml) | |
| Th17 cells + OX40L L-cells + A10 (0.2 μg/ml) | 34.3 |
| Th17 cells + OX40L L-cells + A10 (2 μg/ml) | 22.8 |
| Th17 cells + OX40L L-cells + A10 (20 μg/ml) | 15.4 |
| Th17 cells + OX40L L-cells + B66 (0.2 μg/ml) | 45.2 |
| Th17 cells + OX40L L-cells + B66 (2 μg/ml) | 28.3 |
| Th17 cells + OX40L L-cells + B66 (20 μg/ml) | 20.3 |

Compared to Th1 primed cells, TH17 cells showed significantly higher levels of IL-17 after 7 days of priming naïve CD4⁺ T-cells. After an additional co-culture of Th17 with irradiated OX40L transfected L-cells or parental L-cells, the Th17 cells produced significant levels of IL-17 in the presence of OX40 costimulation compared to Th1 primed cells. This IL17 production was inhibited by chimeric anti-OX40 mAbs A10 and B66 in a dose dependent manner (Table 10) compared to the isotype control G3-519 (IC).

TABLE 10

| | TH-17 | | TH-1 | |
|---|---|---|---|---|
| | IL-17 | | IL-17 | |
| | Conc | SD | Conc | SD |
| | (pg/ml) | (n: 3) | (pg/ml) | (n: 3) |
| CD4⁺ cells + L-cells | 409.7 | 41.1 | 29.8 | 5.4 |
| CD4⁺ cells/OX40L L-cells | 2264.1 | 40.8 | 20.5 | 4.7 |
| CD4⁺ cells/OX40L L-cells + | 2032.7 | 90.0 | 56.1 | 10.7 |
| IC (20 μg/ml) | | | | |
| CD4⁺ cells/OX40L L-cells + | 2054.6 | 254.0 | 41.4 | 8.4 |
| A10 (0.2 μg/ml) | | | | |
| CD4⁺ cells/OX40L L-cells + A10 | 1795.1 | 304.6 | 39.3 | 9.4 |
| (2 μg/ml) | | | | |
| CD4⁺ cells/OX40L L-cells + A10 | 1059.9 | 448.6 | 54.1 | 5.7 |
| (20 μg/ml) | | | | |
| CD4⁺ cells/OX40L L-cells + B66 | 2296.3 | 56.8 | 56.6 | 5.8 |
| (0.2 μg/ml) | | | | |
| CD4⁺ cells/OX40L L-cells + B66 | 1144.0 | 224.5 | 63.1 | 3.7 |
| (2 μg/ml) | | | | |
| CD4⁺ cells/OX40L L-cells + B66 | 1069.5 | 224.5 | 71.5 | 6.9 |
| (20 μg/ml) | | | | |

D. L106 Comparison

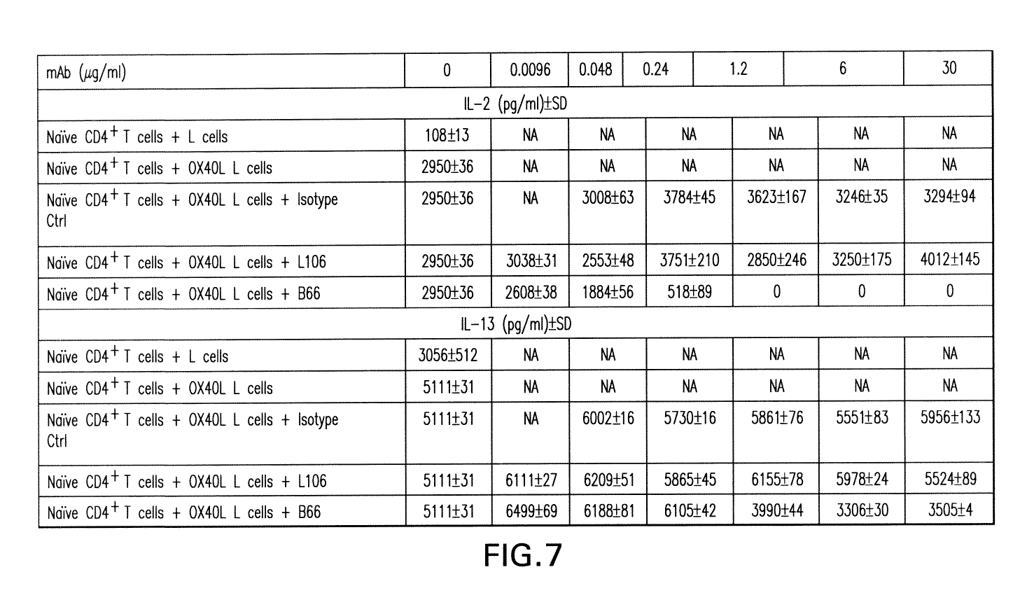

The ability of anti-OX40 mAbs A10 and B66 to inhibit the release of cytokines IL-2 and IL-13 from activated human CD4⁺ T-cells in the presence of OX40 costimulation was compared to commercial anti-OX40 antibody L106 (BD Bioscience, San Jose. CA) using the same protocol in Example 4(B)(2). Inhibition of cytokine production by L106 was not statistically different from the control antibody (See FIG. 7). Hence, the antibody appeared to be non-functional (i.e., no agonistic or antagonistic activity).

### Example 6

### Humanization of an Anti-OX40 mAb

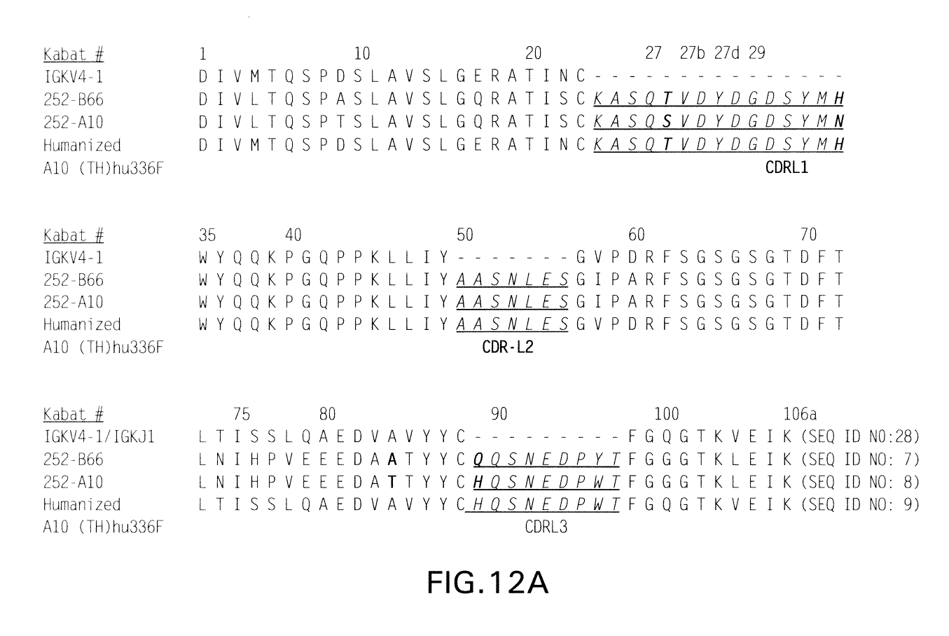

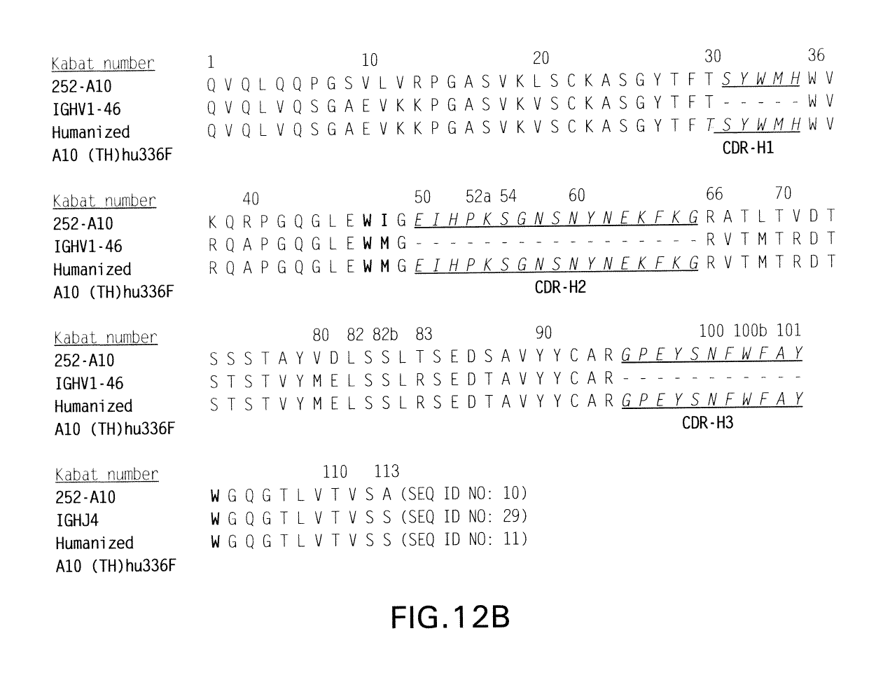

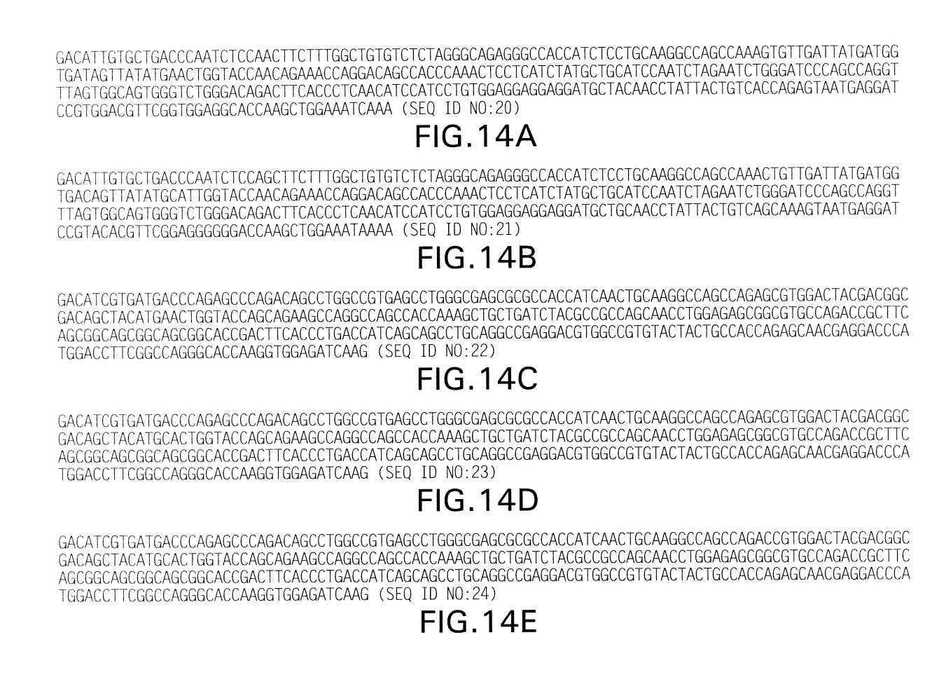

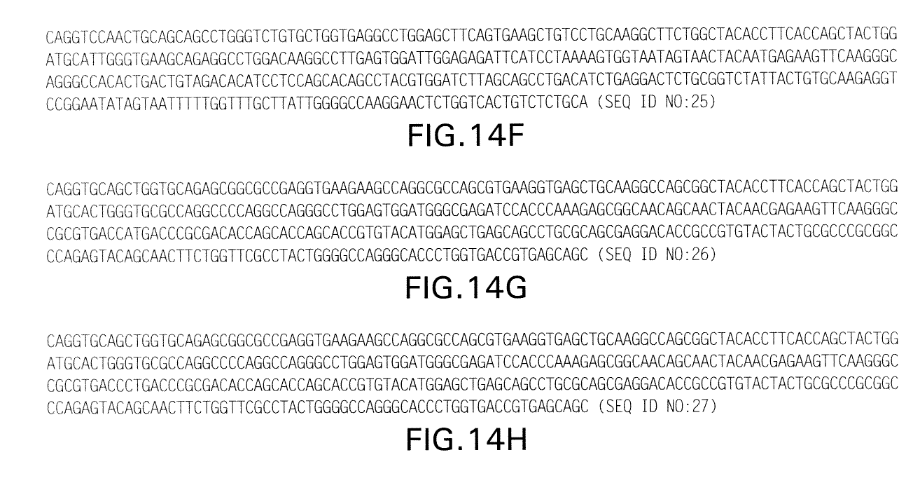

The nucleic acid sequences of the heavy chain variable region (V_{H}) and the light chain variable region (V_{L}) of murine mAb A10 and B66 are depicted in FIG. 14 A-D. FIG. 12 provides the amino acid sequences of the light chain variable region of murine antibodies (derived from DNA sequence) of 252-B66 and 252-A10 compared to the chosen human light chain template. Both antibodies act as OX40 antagonists and compete with one another. These sequences were compared with human antibody germline sequences available in the public databases. Several criteria were used when deciding on a template, including overall length, similar CDR position within the framework, overall homology, size of the CDR, etc. All of these criteria taken together provided a result for choosing the optimal human template as shown in the sequence alignment between A10 and B66 mAb heavy and light chain sequences and the respective human template sequences depicted in FIGS. 12A and B. A chimeric Fab in a phage vector was constructed as a control which combined the variable regions of the murine A10 and the constant region of the human kappa chain and the CHI part of human IgG.

In this case, more than one human framework template was used to design this antibody. The human template chosen for the V_{H} chain was a combination of IGHV1-46 of the subgroup V1 (Gene Bank Accession number X92343) and germ line IGHJ4 (Gene Bank Accession Number J00256) (See FIG. 12B). The human template chosen for the V_{L}, chain was a combination of IGKV4-1 (Gene Bank Accession Number Z00023) and Germ line J template IGHJ1 (Gene Bank Accession Number J00242) (See FIG. 12A). FIG. 12B provides the amino acid sequence of the heavy chain variable region of the murine antibody (derived from DNA sequence), its alignment with selected human germ line templates and the sequence of the humanized variable region of A10(TH)hu336F. The numbering of the amino acid sequence is done according to the method of Kabat. The murine CDRs from A10 were grafted onto a human variable framework. In this sequence, the total number of amino acid residues in the framework is 87; the total number of amino acid residues different between the human and the murine framework is 21, and no murine residues are retained in the framework of the humanized heavy chain.

Sequence comparison revealed that murine antibody B66 differed from A10 in only 4 amino acids in the variable sequence, but functional analysis discussed in Example 4 above showed that B66 had higher OX40 binding and antagonist activity. Two of the amino acid changes in B66 were located in the first CDR and are changes from the mouse germ line sequence. It was reasoned that these changes might be responsible for the enhanced activity of the B66. However, when CDRs from these antibodies were grafted onto a human variable framework, B66 secreted poorly and was therefore, less suitable for further development. Therefore, the CDRL1 of B66 and CDRL2 and CDRL3 of A10 were chosen to graft onto a human variable template to make a humanized anti-OX40 antagonist. By this method, the binding affinity and antagonist activity of the humanized A10 molecule was increased relative to the parent murine antibody.

A. Kinetic Analysis of Anti-OX40 Fab by BiaCore

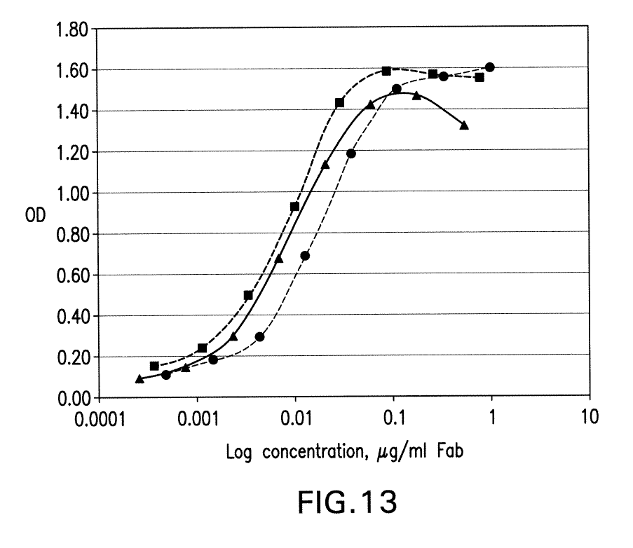

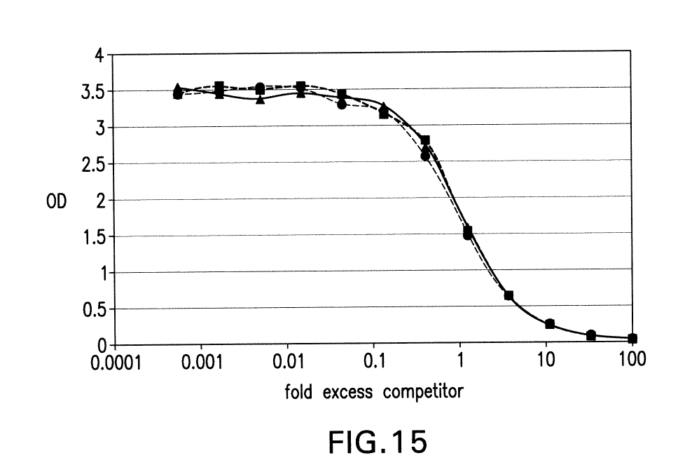

FIG. 13 shows the OX40 binding activity of the resulting molecule compared to the original murine molecule. CDRL1 was also mutated to reflect the original CDRL1 sequence of A10. The replacement of the CDRL1 of A10 with the CDRL1 of B66 resulted in increased binding activity of the ‘humanized’ A10 molecule A10(TH)hu336F. FIG. 15 shows that humanized clone A10(TH)hu336F, murine A10 and its chimeric form compete equally well for OX40.

### Example 7

### Characterization of Humanized Variants of A10(TH)hu336F

Six variants of the humanized A10 candidate A10(TH)hu336F were screened using a binding ELSA, cell-base binding assays and further functional assays demonstrating their antagonistic activity. Two of the 6 humanized A10, the variants A10-D and F were subjected to six functional assays.

The humanized variable regions described above were cloned into mammalian expression vectors such that the humanized variable regions were fused in frame with human constant regions when expressed. Additional humanized candidates were also cloned into mammalian expression vectors to produce whole antibody for testing. These additional candidates include the humanized A10 VL region with the original A10 CDRL1 (A & D), the humanized A10 VL region with Kabat number 34 changed from asparagine (N) to histidine (H) (B & E) (the CDR-L1 in this variant comprises the amino acid sequence KASQTVDYDGDSYMH (SEQ ID NO:61), and an alternative humanized VH region containing a murine leucine (L) at sequential number 70 (A10 L70) (Kabat number 69). Antibodies were expressed in mammalian cells in the combinations shown Table 11. Table 12 shows the corresponding Biacore data. The nucleic acid sequences of the variable regions of these humanized candidates as well as the murine variable regions are presented in FIG. 14.

TABLE 11

| Whole Antibody | Kappa Variable | Heavy Variable |
|---|---|---|
| Name | Candidate | Candidate |
| A | A10 LC (SH), | A10 L70 |
| B | A10 LC (SN) | A10 L70 |
| C | A10 LC (TH) hu 336 | A10 L70 |
| D | A10 LC (SH), | A10 humanized |
| E | A10 LC (SN) | A10 humanized |
| F | A10 LC (TH) hu 336 | A10 humanized |

TABLE 12

| Sample | KD [nM] | ka [M−1s−1] | SE (ka) | kd [s−1] | SE (kd) | χ² |
|---|---|---|---|---|---|---|
| A | 1.38 | 1.31e5 | 452 | 1.79e−4 | 7.44e−7 | 0.257 |
| B | 0.94 | 1.70e5 | 355 | 1.56e−4 | 6.50e−7 | 0.524 |
| C | 1.44 | 1.16e5 | 483 | 1.51e−4 | 8.28e−7 | 0.352 |
| D | 1.21 | 1.40e5 | 508 | 1.70e−4 | 8.55e−7 | 0.319 |
| E | 1.25 | 1.51e5 | 391 | 1.91e−4 | 8.48e−7 | 0.395 |
| F | 1.12 | 1.41e5 | 474 | 1.58e−4 | 7.53e−7 | 0.267 |

A. TCR-activated naïve CD4+ T-cells Assay

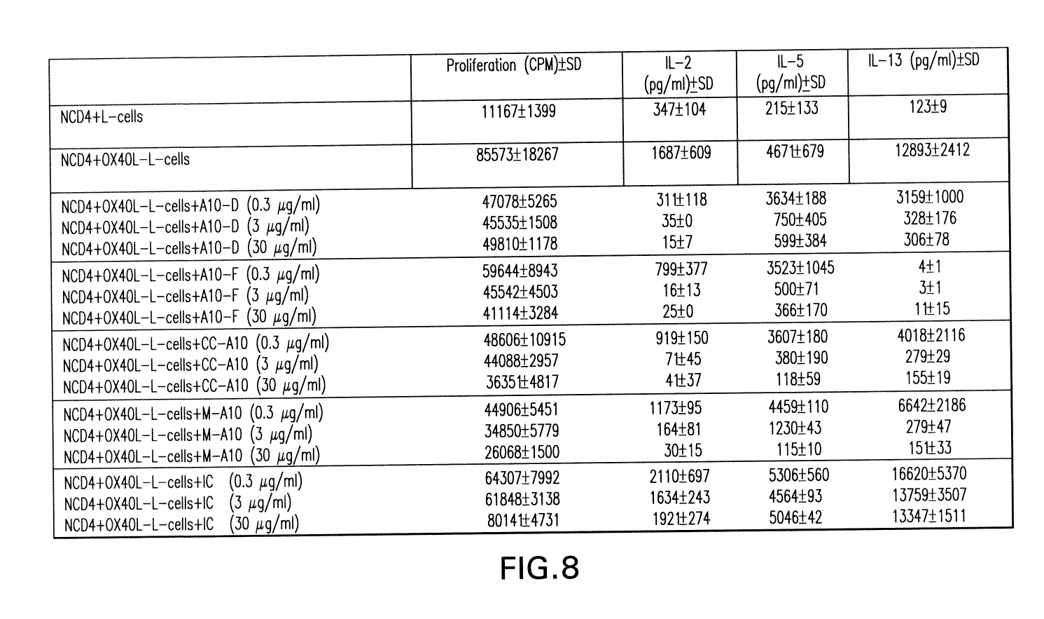

Naïve human CD4⁺ T-cells were activated according to the same protocol described in Example 4 (B)(2) above. After seven days of culture, naïve CD4⁺ T-cells activated in the presence of OX40L produced at least 10 times more IL-2, IL-5 and IL-13 and proliferated significantly more than did those primed in the presence of L-cells (FIG. 8).

The humanized variants A10-D and A10-F inhibited cytokine release (IL-2/IL-5/IL-13) and CD4+ T-cells proliferation to the same level as the murine parent and chimeric forms of A10 as compared to an isotype control (FIG. 8).

B. Primed T-Cell Assays

The humanized variants A10-D and A10-F were tested for their antagonistic activities: inhibiting the proliferation and cytokine release of Th1 primed T-cells, Th2 primed T-cells and Th17 cells in the presence of irradiated OX40L transfected L-cells or parental L-cells (See protocols in Example 5).

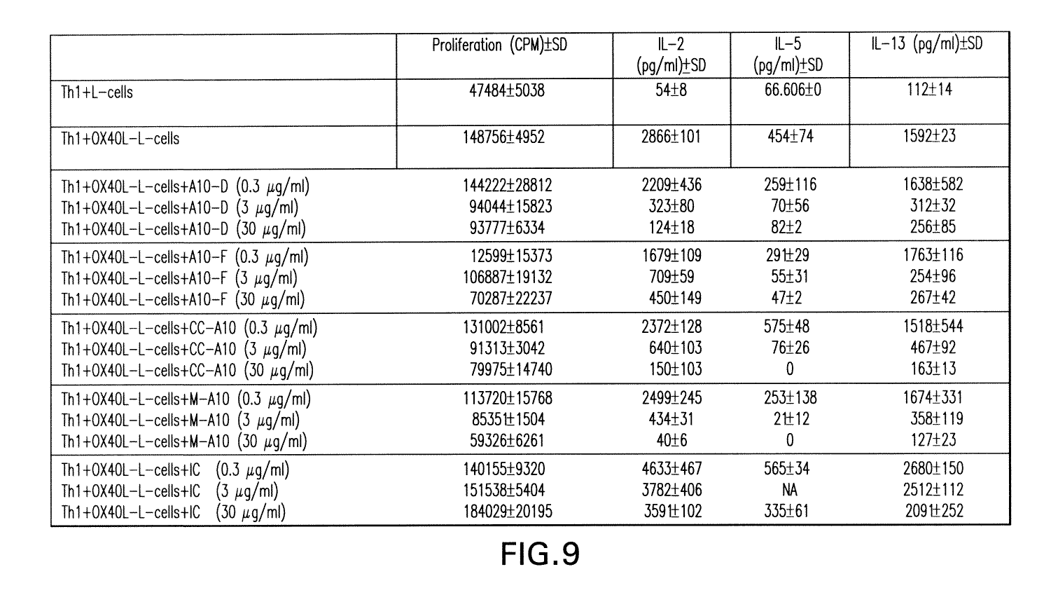

Under Th1 conditions, the humanized variants A10-D and A10-F) inhibited the production of IL-2, IL-5, IL-13 cytokines and cell proliferation to the same degree as the chimeric (CC-A10) and parent mouse (M-A10) mAbs, as compared to the isotype control (FIG. 9).

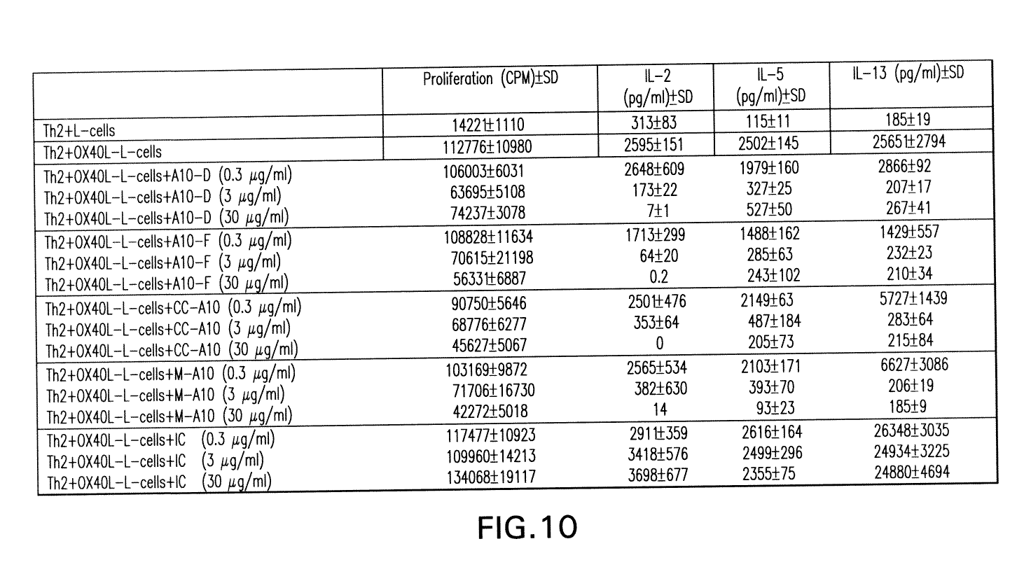

Under Th2 conditions, the humanized variants A10-D and A10-F showed an antagonistic effect on activated CD4+ T-cells by inhibiting their proliferation and cytokine release (IL-2, IL-5, and IL-13). (FIG. 10)

The humanized variants A10 D and A10-F were also tested in parallel with their chimeric and mouse anti-OX40 mAbs in order to compare their antagonist activities on Th17 cell proliferation, IL-17 cytokine production and cell apoptosis. Results are shown in Table 13.

TABLE 13

| | Proliferation | IL-17 | GM-CSF |
|---|---|---|---|
| | (CPM) ± SD | (pg/ml) ± SD | (pg/ml) ± SD |
| Th17 + L-cells | 23997 ± 1715 | 14 ± 9 | 319 ± 290 |
| Th17 + OX40L-L-cells | 167684 ± 581 | 858 ± 205 | 1941 ± 246 |
| Th17 + OX40L-L-cells + A10-D (0.3 μg/ml) | 87715 ± 10048 | 313 ± 76 | 1730 ± 187 |
| Th17 + OX40L-L-cells + A10-D (3 μg/ml) | 11486 ± 1901 | 107 ± 49 | 533 ± 120 |
| Th17 + OX40L-L-cells + A10-D (30 μg/ml) | 12365 ± 3514 | 184 ± 214 | 410 ± 312 |
| Th17 + OX40L-L-cells + A10-F (0.3 μg/ml) | 65957 ± 18160 | 240 ± 37 | 1414 ± 240 |
| Th17 + OX40L-L-cells + A10-F (3 μg/ml) | 10376 ± 2628 | 152 ± 26 | 455 ± 108 |
| Th17 + OX40L-L-cells + A10-F (30 μg/ml) | 9380 ± 2673 | 106 ± 33 | 203 ± 63 |
| Th17 + OX40L-L-cells + CC-A10 (0.3 μg/ml) | 94905 ± 15869 | 278 ± 46 | 1743 ± 303 |
| Th17 + OX40L-L-cells + CC-A10 (3 μg/ml) | 17566 ± 1636 | 135 ± 55 | 626 ± 224 |
| Th17 + OX40L-L-cells + CC-A10 (30 μg/ml) | 9029 ± 2090 | 125 ± 58 | 235 ± 125 |
| Th17 + OX40L-L-cells + M-A10 (0.3 μg/ml) | 106682 ± 8066 | 373 ± 99 | 1550 ± 695 |
| Th17 + OX40L-L-cells + M-A10 (3 μg/ml) | 18797 ± 4807 | 88 ± 9 | 253 ± 23 |
| Th17 + OX40L-L-cells + M-A10 (30 μg/ml) | 6934 ± 997 | 97 ± 48 | 40 ± 16 |
| Th17 + OX40L-L-cells + IC (0.3 μg/ml) | 168442 ± 21659 | 866 ± 218 | 1937 ± 131 |
| Th17 + OX40L-L-cells + IC (3 μg/ml) | 151481 ± 3176 | 862 ± 371 | 1861 ± 295 |
| Th17 + OX40L-L-cells + IC (30 μg/ml) | 149621 ± 6010 | 918 ± 259 | 1575 ± 229 |

Through the identification of apoptotic cells by flow cytometry and Annexin-V staining, the data presented in Table 12 demonstrates that OX40L significantly decreased Th17 apoptosis, whereas humanized variants A10-D and A10-F increased apoptosis in a dose dependent manner, as compared to an isotype control G3-519 treatment (IC) (Table 14).

TABLE 14

| | TH17 |
|---|---|
| | Survival (%) |
| Th17 + L-cells | 20.4 |
| Th17 + OX40L-L-cells | 39.2 |
| Th17 + OX40L-L-cells + A10-D (0.3 μg/ml) | 49.6 |
| Th17 + OX40L-L-cells + A10-D (3 μg/ml) | 18.0 |
| Th17 + OX40L-L-cells + A10-D (30 μg/ml) | 22.3 |
| Th17 + OX40L-L-cells + IC (0.3 μg/ml) | 46.6 |
| Th17 + OX40L-L-cells + IC (3 μg/ml) | 47.1 |
| Th17 + OX40L-L-cells + IC (30 μg/ml) | 44.8 |

C. Th2/TSLP-DCs

CD11c⁺ dendritic cells (DCs) were isolated from the buffy coat of human blood from healthy adult volunteers. The DC-enriched population was obtained from PBMCs by negative immunoselection using a mixture of mAbs against the lineage markers CD3 (OKT3), CD14 (M5E2), CD15 (HB78), CD20 (L27), CD56 (B159), and CD235α (10F7MN). The population was passed through goat anti-mouse IgG-coated magnetic beads (M-450; Miltenyi Biotec; CA) to remove the bound cells. The CD11c⁺ lineage and CD4⁺ cells were isolated by a FACS Aria (using allophycocuanin (APC)-labeled anti-CD11c (B-ly6), a mixture of FITC-labeled mAbs CD19 (HIB19,) and CD56 (NCAM16.2,), and APC-Cy7-labeled CD4 (RPA-T4,) to reach >99% purity. CD11c⁺ DCs were cultured in Yssel's medium containing 2% human AB serum (Gemini Bio-Products, Woodland, Calif.). Cells were seeded at a density of 2×10⁵ cells/200 ml medium in flat-bottomed 96-well plate in the presence of 15 ng/ml of TSLP (recombinant human TSLP generously gifted by Dr. Yun-Jun Liu. M.D. Anderson Cancer Center) for 24 hours.

Allogeneic CD4⁺ CD45RA⁺ naïve T-cells (purity>99%) were isolated using CD4⁺ T-cells isolation kit II (Miltenyi Biotec, CA) followed by cell sorting (as a CD4⁺ CD45RA⁺ CD45RO− CD25⁻ fraction). The cells were co-cultured with washed TSLP-stimulated myeloid dendritic cells (mDCs) (DCT-cell ratio, 1:5) in round-bottomed 96-well culture plate for 7 days. After one cycle of stimulation (7 days) by the mDCs, the primed CD4⁺ T-cells were collected and washed.

For detection of cytokine production in the culture supernatants, the T-cells were re-stimulated with plate-bound 5 μg/ml anti-CD3 and 1 μg/ml soluble anti-CD28 at a concentration of 10⁶ cells/ml for 48 hours. The levels of IL-5, IL-13 and TNF-α(were measured by ELISA. Cell proliferation and cytokine production in Th2 cells primed with TSLP-activated myeloid DCs were inhibited in the presence of humanized variant A10-F, as compared to an isotype control antibody (Table 15).

TABLE 15

| | Proliferation | IL-5 | IL-13 | TNF-α |
|---|---|---|---|---|
| | (CPM) ± SD | (pg/ml) ± SD | (pg/ml) ± SD | (pg/ml) ± SD |
| Th2 | 2745 ± 31 | 0 | 0 | 0 |
| Th2 + TSLP-DCs | 195546 ± 17034 | 2188 ± 560 | 5107 ± 934 | 456 ± 50 |
| Th2 + TSLP-DCs + A10-F (0.12 μg/ml) | 95335 ± 13716 | 2109 ± 811 | 1973 ± 289 | 174 ± 14 |
| Th2 + TSLP-DCs + A10-F (6 μg/ml) | 70750 ± 19116 | 509 ± 93 | 1943 ± 68 | 95 ± 8 |
| Th2 + TSLP-DCs + A10-F (60 μg/ml) | 56792 ± 2993 | 384 ± 133 | 1728 ± 243 | 93 ± 75 |
| Th2 + TSLP-DCs + IC (0.12 μ/ml) | 191760 ± 11838 | 3271 ± 652 | 5341 ± 1184 | 543 ± 59 |
| Th2 + TSLP-DCs + IC-F (6 μg/ml) | 211124 ± 16370 | 1885 ± 281 | 4887 ± 673 | 392 ± 36 |
| Th2 + TSLP-DCs + IC (12 μg/ml) | 213641 ± 14303 | 2261 ± 116 | 5632 ± 356 | 471 ± 75 |

For intracellular cytokine production, the primed CD4⁺ T-cells were stimulated with 50 ng/ml of PMA plus 2 μg/ml of ionomycin for 6 hours. Ten μg/ml brefeldin A (eBioscience, San Diego, Calif.) was added during the final 2 hours. The cells were stained with the combination of PE-labeled mAbs to IL-2 and IL-13, and FITC-labeled anti IFN-γ or anti-TNF-α using Fixation and Permeabilization buffers (eBioscience, CA). IL-2, IL-13 and TNF-α cytokine production in Th2 cells primed with TSLP-activated myeloid DCs were inhibited in the presence of humanized variant A10-F, as compared to an isotype control antibody (Table 16).

TABLE 16

| | IL-2 | IL-13 | TNF-α |
|---|---|---|---|
| | % fluorescent | % fluorescent | % fluorescent |
| | cells | cells | cells |
| Th2 | NA | NA | NA |
| Th2 + TSLP-DCs | 71 | 11 | 44 |
| Th2 + TSLP-DCs + | 63 | 10 | 31 |
| A10-F (0.12 μg/ml) | | | |
| Th2 + TSLP-DCs + | 57 | 5 | 30 |
| A10-F (6 μg/ml) | | | |
| Th2 + TSLP-DCs + | 57 | 5 | 28 |
| A10-F (60 μg/ml) | | | |
| Th2 + TSLP-DCs + | 72 | 15 | 53 |
| IC (0.12 μg/ml) | | | |
| Th2 + TSLP-DCs + | 73 | 13 | 54 |
| IC (6 μg/ml) | | | |
| Th2 + TSLP-DCs + | 69 | 16 | 49 |
| IC (12 μg/ml) | | | |

The OX40 costimulation increased the survival of primed Th2 cells as shown by a decrease in the number of CD4+ cells stained with annexin and an increased number of CD4+ cells with intracellular Survivin protein expression. However, the addition of humanized variant A10-F to the cell culture induced a significant increase in apoptosis and a lower expression of Survivin (Table 17).

TABLE 17

| | Annexin | |
|---|---|---|
| | staining (%) | Survivin (%) |
| Th2 | NA | NA |
| Th2 + TSLP-DCs | 12 | 90 |
| Th2 + TSLP-DCs + A10-F (0.12 μg/ml) | 24 | 86 |
| Th2 + TSLP-DCs + A10-F (6 μg/ml) | 29 | 76 |
| Th2 + TSLP-DCs + A10-F (60 μg/ml) | 35 | 70 |
| Th2 + TSLP-DCs + IC (0.12 μg/ml) | 14 | 90 |
| Th2 + TSLP-DCs + IC-F (6 μg/ml) | 14 | 86 |
| Th2 + TSLP-DCs + IC (12 μg/ml) | 14 | 90 |

D. Th1/LPS-DCs Protocol

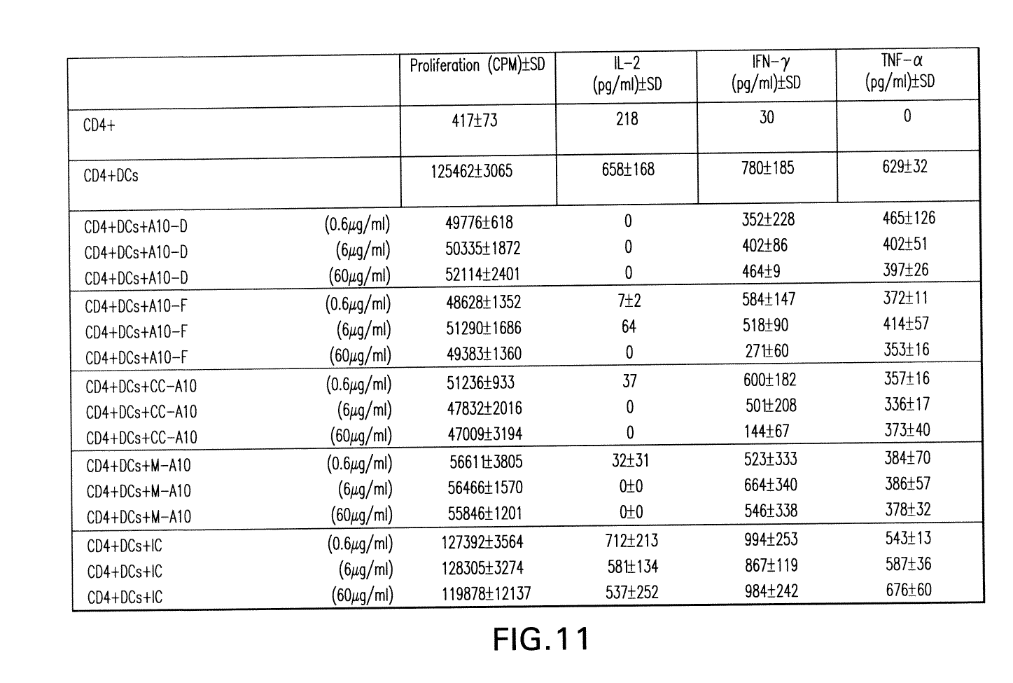

To analyze T-cell polarization toward a Th1 profile, naïve CD4 T-cells were co-cultured with allogeneic monocyte-derived DCs that had been stimulated with LPS for 24 hours. DCs were generated from human PBMCs by standard protocol well known in the art. The low density PBMCs were harvested and CD14+ cells were positively selected using CD14-microbeads and AutoMacs equipment (Miltenyi Biotec, Auburn, Calif.). To induce DC differentiation, 10⁶ cells/ml of CD14+ monocytes were cultured in complete medium (RPMI-10% FBS), 500 IU/ml human rGM-CSF (R&D Systems. MN), and 400 IU/ml human rIL-4 (R&D Systems, MN) at 37° C. under 5% CO2. On day 2 or 3, the DC cultures received an additional dose of GM-CSF and IL-4. On day 5, non-adherent DCs were harvested by gentle pipetting and re-cultured with 1 μg/ml of LPS (Sigma-Aldrich, MO). LPS-activated DCs were harvested, washed, and used for priming naïve CD4 T-cells into Th-1 polarized T-cells. The cells were incubated in a 96-well plate at a DC/T-cell ratio of 1:4. The humanized variants A10-D and A10-F were also tested for their antagonistic activities; and the ability to inhibit the proliferation and cytokine production of Th1 primed T-cells in the presence of LPS-activated DCs. The humanized variants A10-D and F inhibited the IL-2, IFN-γ, and TNF-α cytokines production and cell proliferation to the same degree as the chimeric (CC-A10) and mouse (M-A10) variant mAbs, as compared to an isotype control (FIG. 11). The inhibition of cell proliferation and IFN-γ and TNF-α cytokines production was not total because the LPS-activated DCs can provide other co-stimulatory signals other than OX40.

E. Cross-Reactivity Assays

According to the literature published, the shared homology between human and mouse OX40 protein is considered low. Consistent with this, chimeric A10 and B66 did not show any cross reactivity toward mouse OX40 protein as compared to an isotype control (IC: 2C4) in ELISA platform (Table 18).

TABLE 18

| | Mouse OX40 Fc (ng/ml) | | | | Human OX40 Fc (ng/ml) | | | |
|---|---|---|---|---|---|---|---|---|
| OD450 | 0 | 3.9 | 62.5 | 250 | 0 | 3.9 | 62.5 | 250 |
| B66 | 0.134 | 0.034 | 0.039 | 0.045 | 0.02 | 1.225 | 2.859 | 3.377 |
| A10 | 0.025 | 0.04 | 0.035 | 0.035 | 0.019 | 1.308 | 2.586 | 2.832 |
| IC (2C4) | 0.035 | 0.046 | 0.078 | 0.294 | 0.02 | 0.031 | 0.027 | 0.031 |
| anti- | 0.051 | 2.486 | 4.0 | 4.0 | 0.026 | 0.047 | 0.046 | 0.083 |
| mouseOX40Ab | | | | | | | | |

Therefore, a flow cytometry staining was used to test the anti-OX40 mAbs in a preclinical animal model setting to determine if they cross-react with monkey OX40 protein. Total CD4⁺ T-cells were isolated from total blood of Rhesus and Cynomolgus-Monkey and from human buffy coat using CD4 micro-beads (Miltenyi Biotec, Auburn, Calif.). The isolated CD4⁺ T-cells were activated for 2 days using 5 μg/ml of pre-coated anti-CD3 mAb (SP34, capable of cross-reacting to human and both monkey species Rhesus and Cynomolgus CD3 protein) and 1 μg/ml of soluble anti-CD28 mAb (CD28.2, capable of cross-reacting to human and both monkey species Rhesus and Cynomolgus CD28 protein,). The cells were subjected to surface staining using antibodies that cross-react to monkey and human CD4 (L200) and CD25 (M-A251) proteins. For OX40 cell surface expression, humanized A10-F OX40 mAb conjugated to PE were used. The data analysis showed that A10-F readily recognized human, Rhesus, and Cynomolgus activated CD4 T-cells (Table 19).

TABLE 19

| | % of activated CD4+ T- |
|---|---|
| | cells recognized |
| Human | 70 |
| Cynomolgus Monkey | 88 |
| Rhesus Monkey | 75 |

### Example 8

### Epitope Mapping

The OX40 epitope to which A10(TH)hu336F binds is mapped using two standard approaches to epitope mapping, yeast surface display and construction of chimeric molecules. In yeast surface display, the extra-cellular domain of OX40 is expressed in Saccharomyces cerevisiae as a protein fusion that is capable of being secreted and displayed on the surface of the yeast. Random mutagenesis of the OX40 expression vector allows libraries of OX40 mutant proteins to be expressed on the yeast surface. Mutants that express protein (as determined by the presence of a C-terminal tag) that fail to bind OX40 can be stained using fluorescent antibodies and isolated by FACS. The mutant expression plasmids are isolated from the rescued yeast and sequenced to determine which amino acid residues are required for A10(TH)hu336F—OX40 binding.

The second epitope mapping approach relies on the observation that A10(TH)hu336F binds to human but not murine OX40. Chimeric OX40 molecules are constructed that have regions of the human OX40 replaced with homologous regions of murine OX40. The chimeric molecules are expressed on the surface of mammalian cells and binding to fluorescently labeled A10(TH)hu336F is monitored by FACS. The OX40 binding epitope is determined by analyzing which regions of human OX40 are required for A10(TH)hu336F binding.

Those skilled in the art will recognize, or be able to ascertain using no more than routine experimentation, many equivalents to the specific embodiments of the invention described herein. Such equivalents are intended to be encompassed by the following claims.

1. An isolated antagonist antibody that specifically binds to a human OX40 epitope, said antibody comprising:
a. a heavy chain variable region (VH) comprising:
i. a VH CDR1 having the amino acid sequence of SEQ ID NO:17;
ii. a VH CDR2 having the amino acid sequence of SEQ ID NO:18; and/or
iii. a VH CDR3 having the amino acid sequence of SEQ ID NO:19; and/or
b. a light chain variable region (VL) comprising:
i. a VL CDR1 having the amino acid sequence of SEQ ID NO:12, 15 or 61;
ii. a VL CDR2 having the amino acid sequence of SEQ ID NO:13; and/or
iii. (3) a VL CDR3 having the amino acid sequence of SEQ ID NO:14 or 16.

2. The antibody of claim 1, wherein the antibody comprises:
a. a heavy chain variable region (VH) comprising:
i. a VH CDR1 having the amino acid sequence of SEQ ID NO:17;
ii. a VH CDR2 having the amino acid sequence of SEQ ID NO:18; and
iii. a VH CDR3 having the amino acid sequence of SEQ ID NO:19; and/or
b. a light chain variable region (VL) comprising:
i. a VL CDR1 having the amino acid sequence of SEQ ID NO:12;
ii. a VL CDR2 having the amino acid sequence of SEQ ID NO:13; and
iii. (3) a VL CDR3 having the amino acid sequence of SEQ ID NO:14.

3. The antibody of claim 1, wherein the antibody comprises:
a. a heavy chain variable region (VH) comprising:
i. a VH CDR1 having the amino acid sequence of SEQ ID NO:17;
ii. a VH CDR2 having the amino acid sequence of SEQ ID NO:18; and
iii. a VH CDR3 having the amino acid sequence of SEQ ID NO:19; and/or
b. a light chain variable region (VL) comprising:
i. a VL CDR1 having the amino acid sequence of SEQ ID NO:12;
ii. a VL CDR2 having the amino acid sequence of SEQ ID NO:13; and
iii. a VL CDR3 having the amino acid sequence of SEQ ID NO:16.

4. The antibody of claim 1, wherein the antibody comprises:
a. a heavy chain variable region (VH) comprising:
i. a VH CDR1 having the amino acid sequence of SEQ ID NO:17;
ii. a VH CDR2 having the amino acid sequence of SEQ ID NO:18; and
iii. (3) a VH CDR3 having the amino acid sequence of SEQ ID NO:19; and/or
b. a light chain variable region (VL) comprising:
i. a VL CDR1 having the amino acid sequence of SEQ ID NO:15;
ii. a VL CDR2 having the amino acid sequence of SEQ ID NO:13; and
iii. (3) a VL CDR3 having the amino acid sequence of SEQ ID NO:14.

5. The antibody of claim 1, wherein the antibody comprises:
a. a heavy chain variable region (VH) comprising:
i. a VH CDR1 having the amino acid sequence of SEQ ID NO:17;
ii. a VH CDR2 having the amino acid sequence of SEQ ID NO:18; and
iii. a VH CDR3 having the amino acid sequence of SEQ ID NO:19; and/or
b. a light chain variable region (VL) comprising:
i. a VL CDR1 having the amino acid sequence of SEQ ID NO:15;
ii. a VL CDR2 having the amino acid sequence of SEQ ID NO:13; and a VL CDR3 having the amino acid sequence of SEQ ID NO:16.

6. The antibody of claim 1, wherein the antibody comprises:
a. a heavy chain variable region (VH) having the amino acid sequence depicted in any one of SEQ ID NOS:10 or 11 and/or
b. a light chain variable region (VL) having the amino acid sequence depicted in any one of SEQ ID NOS:7, 8, or 9.

7. The antibody of claim 1, wherein the antibody comprises:
a. a heavy chain variable region (VH) encoded by any one of the nucleic acid sequence of SEQ ID NO:25, 26 or 27; and/or
b. a light chain variable region (VL) encoded by any one of the nucleic acid sequence of SEQ ID NO:20, 21, 22, 23, or 24.

8. The antibody of claim 6, wherein the antibody comprises:
a. a VH having the amino acid sequence depicted in SEQ ID NO:10 and a VL having the amino acid sequence depicted in any one of SEQ ID NO: 7 or 8; or
b. a VH having the amino acid sequence depicted in SEQ ID NO:11 and a VL having the amino acid sequence depicted in SEQ ID NO: 9.

9. The antibody of claim 7, wherein the antibody comprises:
a. a VH encoded by the nucleic acid sequence of SEQ ID NO:25 and a VL encoded by the nucleic acid sequence of SEQ ID NO:20 or 21;
b. a VH encoded by the nucleic acid sequence of SEQ ID NO:26 and a VL encoded by the nucleic acid sequence of SEQ ID NO:22, 23 or 24; or
c. a VH encoded by the nucleic acid sequence of SEQ ID NO:27 and a VL encoded by the nucleic acid sequence of SEQ ID NO:22, 23 or 24.

10. The antibody of claim 8, wherein the VH comprises the amino acid sequence depicted in SEQ ID NO:10 and the VL comprises the amino acid sequence depicted in SEQ ID NO: 7.

11. The antibody of claim 8, wherein the VH comprises the amino acid sequence depicted in SEQ ID NO:10 and the VL comprises the amino acid sequence depicted in SEQ ID NO: 8.

12. The antibody of claim 8, wherein the VH comprises the amino acid sequence depicted in SEQ ID NO:11 and the VL comprises the amino acid sequence depicted in SEQ ID NO: 9.

13. The antibody of claim 1 further comprising a constant region.

14. The antibody of claim 13, further comprising an antibody constant region comprising a CH1, CH2 or CH3 domain.

15. The antibody of claim 14 wherein the constant region is from an IgG antibody.

16. The antibody of claim 15, wherein the IgG antibody is an IgG1 antibody, an IgG2 antibody, an IgG3 antibody, or an IgG4 antibody.

17. The antibody of claim 1 wherein the antibody is a human antibody.

18. The antibody of claim 1 wherein the antibody is a chimeric antibody, humanized antibody, monoclonal antibody, or a recombinant antibody.

19. The antibody of claim 1 wherein the antibody is a Fab, Fab′, F(ab′)₂, Fd, single-chain Fv, single-chain antibody, disulfide-linked Fv, single domain antibody, antigen binding fragment, diabody, triabody, or minibody.

20. The antibody of claim 1 further comprising a label.

21. The antibody of claim 1 further comprising a cytotoxin or immunotoxin.

22. An isolated nucleic acid molecule comprising a nucleotide sequence that encodes an antibody of claim 1.

23. A vector comprising the nucleic acid molecule of claim 22.

24. A host cell comprising the vector of claim 23.

25. A composition comprising an antibody of claim 1 and at least one of a physiologically acceptable carrier, diluent, excipient, and stabilizer.

26. A method of treating an OX-40-mediated disorder in a patient, comprising administering to the patient an effective amount of an antibody according to claim 1 wherein said antibody blocks binding of OX40L to OX40 and/or inhibits one or more functions associated with binding of OX40L to OX40.

27. The method of claim 26 wherein the OX40 mediated disorder is an inflammatory or autoimmune disease.

28. The method of claim 27, wherein the disease is allergy, asthma, or diseases associated with autoimmunity and inflammation, such as multiple sclerosis, rheumatoid arthritis, inflammatory bowel disease, graft-versus-host disease, experimental autoimmune encephalomyelitis (EAE), autoimmune neuropathies, autoimmune ureitis, autoimmune hemolytic anemia, autoimmune thrombocytopenia, myasthenia gravis, experimental leishmaniasis, collagen-induced arthritis, colitis (such as ulcerative colitis), contact hypersensitivity reactions, diabetes, Crohn's Disease, Grave's Disease, sarcoidosis, pernicious anemia, temporal artertis, anti-phospholipid syndrome, vasculitides (such as Wegener's granulomatosis), Behcet's disease, dermatitis herpetiformis, pemphigus vulgaris, vitiligo, primary biliary cirrhosis, autoimmune hepatitis, Hashimoto's thyroiditis, autoimmune oophoritis and orchitis, autoimmune disease of the adrenal gland, systemic lupus erythematosis, scleroderma, dermatomyositis, polymysitis, dermatomyositis, spondyloarthropathies (such as ankylosing spondylitis), Reiter's Syndrome, or Sjogren's syndrome.

29. The method of claim 27, wherein the disease is allergy, asthma, or diseases associated with autoimmunity and inflammation, such as multiple sclerosis, rheumatoid arthritis, inflammatory bowel disease, graft-versus-host disease, experimental autoimmune encephalomyelitis (EAE), experimental leishmaniasis, collagen-induced arthritis, colitis (such as ulcerative colitis), contact hypersensitivity reactions, diabetes, Crohn's Disease, or Grave's Disease.

30. The method of claim 26 wherein the antibody is administered to the patient via one or more of the routes including intravenous, intraperitoneal, inhalation, intramuscular, subcutaneous and oral routes.

31. A method for treating a disorder by inhibiting IgE antibody production in a patient, comprising administering to the patient an effective amount of an antibody according to claim 1.

32. The method of claim 31 wherein the disorder is bronchial asthma, allergic rhinitis, allergic dermatitis, anaphylaxis, uticaria or atopic dermatitis.

33. A method for reducing the severity of asthma in a mammal, comprising administering to the mammal an effective amount of an antibody according to claim 1 wherein said antibody comprises at least one of the following properties: (a) binds human OX40 with a K_{D} between about 1×10⁻¹⁰ to about 1×10⁻¹²M; (b) inhibits one or more functions associated with binding OX40; and (c) inhibits binding of OX40L to OX40.

34. The method of claim 33 wherein the antibody binds human OX40 with a KO between about 1×10⁻¹² to about 1×10⁻¹¹M, 1×10⁻¹¹ to about 1×10⁻¹⁰M, 1×10⁻¹⁰ to about 1×10⁻⁹M, 1×10⁻⁹ to about 1×10⁻⁸M, or 1×10⁻⁸ to about 1×10⁻⁷M.

35. A method of producing an antibody comprising culturing the cell of claim 24 under conditions suitable for the production of the antibody, and isolation of the antibody.

36. A hybridoma that produces an antibody as claimed in claim 1.

37. Use of the antibody of claim 1 in the preparation of a medicament.

38. Use of the antibody of claim, 1 for the treatment of a OX40 related disease or disorder.

39. The use according to claim 38, wherein the disease is allergy, asthma, or diseases associated with autoimmunity and inflammation, such as multiple sclerosis, rheumatoid arthritis, inflammatory bowel disease, graft-versus-host disease, experimental autoimmune encephalomyelitis (EAE), autoimmune neuropathies, autoimmune ureitis, autoimmune hemolytic anemia, autoimmune thrombocytopenia, myasthenia gravis, experimental leishmaniasis, collagen-induced arthritis, colitis (such as ulcerative colitis), contact hypersensitivity reactions, diabetes, Crohn's Disease, Grave's Disease, sarcoidosis, pernicious anemia, temporal artertis, anti-phospholipid syndrome, vasculitides (such as Wegener's granulomatosis), Behcet's disease, dermatitis herpetiformis, pemphigus vulgaris, vitiligo, primary biliary cirrhosis, autoimmune hepatitis, Hashimoto's thyroiditis, autoimmune oophoritis and orchitis, autoimmune disease of the adrenal gland, systemic lupus erythematosis, scleroderma, dermatomyositis, polymysitis, dermatomyositis, spondyloarthropathies (such as ankylosing spondylitis), Reiter's Syndrome, or Sjogren's syndrome.

40. The use according to claim 39, wherein the disease is allergy, asthma, or diseases associated with autoimmunity and inflammation, such as multiple sclerosis, rheumatoid arthritis, inflammatory bowel disease, graft-versus-host disease, experimental autoimmune encephalomyelitis (EAE), experimental leishmaniasis, collagen-induced arthritis, colitis (such as ulcerative colitis), contact hypersensitivity reactions, diabetes, or Crohn's Disease.

41. Use of the antibody of claim 1 to detect OX40.

42. A method for the detection of OX40 in a sample, comprising contacting the sample with the antibody of claim 20.

43. A kit comprising the antibody of claim 1 in a predetermined amount in a container, and a buffer in a separate container.

44. A kit comprising the composition of claim 25 in a predetermined amount in a container, and a buffer in a separate container.

45. A medical device comprising the antibody of claim 1.

46. The medical device of claim 45 which is an inhalation device.

47. A medical device comprising the composition of claim 25.

48. The medical device of claim 47 which is an inhalation device.
